(19) United States
(12) Patent Application Publication
(10) Pub. No.: US 2025/0090574 A1
(43) Pub. Date: Mar. 20, 2025

(54) BIOMATERIAL COMPRISING POLY(ITACONATE-CO-DODECANEDIOL)

(71) Applicants: Locke DAVENPORT HUYER, Halifax (CA); Kaitlyn WOODWORTH, Halifax (CA); Neal CALLAGHAN, Halifax (CA)

(72) Inventors: Locke DAVENPORT HUYER, Halifax (CA); Kaitlyn WOODWORTH, Halifax (CA); Neal CALLAGHAN, Halifax (CA)

(21) Appl. No.: 18/923,333

(22) Filed: Oct. 22, 2024

Related U.S. Application Data
(63) Continuation-in-part of application No. 18/143,026, filed on May 3, 2023.
Application No. 18/143,026 is a continuation of application No. 16/927,576, filed on Jul. 13, 2020, now Pat. No. 11,666,598.
(60) Provisional application No. 62/872,878, filed on Jul. 11, 2019.

(51) Int. Cl. A61K 31/765; A61K 9/00; A61K 31/047; A61L 27/38; A61P 29/00; A61P 31/04; C08L 67/02

(52) U.S. Cl. CPC A61K 31/765; A61K 9/0024; A61K 31/047; A61L 27/3839; A61P 29/00; A61P 31/04; C08L 67/02; A61K 2123/00; C08L 2312/06

(57) ABSTRACT

A particle comprising a polyester biomaterial comprising diol monomers and itaconate, wherein the diol monomers comprise at least 1,12-dodecanediol. A method of fabricating the polyester biomaterial comprising the steps of: forming a polyester backbone including the diol monomers; and reacting the polyester backbone with the itaconate monomers. A method of treating at least one of an infection or an inflammation, or a condition resulting directly from an infection or an inflammation, in a subject, the method comprising: providing a polyester biomaterial comprising diol monomers and at least first carboxylate monomers, wherein the first carboxylate monomers are itaconate and the diol monomers comprise at least 1,12-dodecanediol, and wherein the polyester biomaterial is in the form of a particle; and administering the polyester biomaterial to the subject.

### CROSS-REFERENCE TO RELATED APPLICATIONS

This application is a continuation-in-part of U.S. patent application Ser. No. 18/143,026 filed May 3, 2023, which is a continuation of U.S. patent application Ser. No. 16/927,576 filed Jul. 13, 2020, which claims priority to and benefit of U.S. Provisional Patent Application No. 62/872,878 filed Jul. 11, 2019, the contents of which are all incorporated by reference herein in their entireties.

### FIELD

The present disclosure relates to a particle comprising a polymer biomaterial. In particular, the disclosure relates to a particle comprising polyester biomaterial containing itaconate (ITA) and at least 1,12-dodecanediol, and a method of treating infection and/or inflammation by administering the polyester biomaterial containing itaconate (ITA) and 1,12-dodecanediol to a subject in the form of a particle.

### BACKGROUND

The use of polymer-based biomaterial implants in healthcare has been extensively reported¹. Many of these devices are subject to limited efficacy due to poor interaction with host immunity², development of surgical site infection³, or cell toxicity. Technological development of materials with inherent bioactivity to combat these limitations has been notable, but the complexity of the cell-material microenvironment often leads to beneficial outcomes in a single functionality with detrimental impact on others^{3, 4}. Biomaterials designed in a biomimetic approach to support implant integration, minimize local infection, and remain non-toxic to the body would be ideal.

Under glucose deprived conditions, ITA is a potent inhibitor of isocitrate lyase (ICL)^{10, 11}, a key enzyme in the microorganism glyoxylate shunt that is often attributed to the resistivity of organisms¹². ITA enables the innate immunity to control bacterial presence and modulate local inflammation without exerting damage to the surrounding tissue, roles which represent exact design criteria for the biomaterial of the present disclosure.

Growth inhibition has been observed on a number of bacterial strains in the presence of ITA, including Myobacterium tuberculosis¹³, methicillin resistant Staphyloccus aureus (MRSA)¹⁴ and multidrug resistant Acinetobacter baumanali⁴. Initially considered primarily as an antibacterial response in innate immunity, ITA has emerged recently as a mechanistic regulator of macrophage inflammation¹⁵.

Macrophages embody a central role in inflammatory control, capable of initiating inflammation via the secretion of pro-inflammatory cytokines and chemokines in response to pathogens, and conversely regulating the pathogenesis of inflammation by transitioning their phenotype¹⁶. Given the central role of macrophages in non-communicable diseases (NCDs), current immunomodulation strategies often involve drug or cytokine targeting macrophages that directly modulate immune-related signaling processes. However, both local and systemic immunomodulatory therapies generally struggle to balance efficacy to adverse effects including toxicity and immunosuppression; these difficulties are due in part to low specificity in cell targets, difficulty in fine-tuning macrophage phenotype due to the broadness of pharmaceutical mechanism, and in obstacles in effective therapeutic delivery to specific tissues¹⁷. There is a need for novel specific strategies to control macrophage-directed inflammation¹⁸.

In response to inflammatory stimuli, such as LPS and type I and II interferons (IFN), macrophages reprogram their metabolic profile, characterized significantly by the accumulation of succinate¹⁹, which kinetically downregulates TCA flux and forces glycolytic upregulation, while also maintaining reactive oxygen species (ROS) production via its succinate dehydrogenase (SDH)-mediated oxidation²⁰. SDH further stabilizes HIF-1α and promotes the production of pro-inflammatory IL-1β, which further impacts inflammation-related gene expression²¹. Metabolic rewiring also generates significant concentrations of ITA, a small carboxylic acid metabolite that serves as an intrinsic anti-inflammatory metabolite regulator of inflammation²². The central immunomodulatory activity of ITA lies in its metabolic regulation capability, particularly within macrophages under inflammatory conditions. ITA directly inhibits SDH, which suppresses succinate oxidation-induced ROS generation, which is a crucial component of classical pro-inflammatory signaling²³. ITA further antagonizes pro-inflammatory macrophage metabolic reorganization by inhibiting multiple key glycolytic enzymes including aldolase A (ALDOA), glyceraldehyde-3-phosphate dehydrogenase (GAPDH), and lactate dehydrogenase A (LDHA) through cysteine modifications²⁴, while enhancing TCA activity by upregulating glutamate oxidation²⁵. Finally, ITA also mitigates of inflammation-associated oxidative through the stabilization of erythroid 2-reated factor 2 (Nrf2) activation and the regulation of electrophilic stress responses along the IκBζ-ATF3 axis resulting in decreased IL-6 expression in an Nrf2-independent mechanism²⁶.

The multimodal behavior of ITA presents opportunity for its role as a therapeutic and inspires synthesis of a polymer that would facilitate multiple roles in a similar manner that cells do. For example, the established mechanisms of ITA as an intrinsic immunomodulator highlights its potential as a biomimetic therapy in chronic inflammatory conditions. Unfortunately, the polarity of ITA makes it challenging to passively diffuse through cell membranes, necessitating specific transport mechanisms or an innovative delivery system. The immunomodulatory potential of ITA has been investigated using membrane-permeable ITA derivatives, such as dimethyl itaconate (DMI) and 4-octyl itaconate (4OI), which have demonstrated their ability to inhibit the expression of pro-inflammatory mediators like IL-6, IL-12, TNF-α and IL-1β in LPS-stimulated macrophages²⁴. However, exogenous ITA derivatives do not recapitulate the full electrophilic stress response and transcriptional profile of endogenous ITA²⁸. Furthermore, the administration of ITA derivatives does not enhance intracellular ITA levels²⁹, whereas non-modified ITA accumulates in both resting and activated macrophages²⁸. This suggests that natural ITA has a distinct behavior compared to its electrophilic derivatives, with more complex immunomodulatory functions beyond simple immunosuppression. Furthermore, the bioavailability of ITA remains a challenge, especially when administered orally, as it is quickly removed from circulation³⁰.

Controlled and targeted drug delivery offers advantages such as maintaining therapeutic drug concentrations, reducing systemic toxicity, localizing delivery to the target site, and protecting therapeutic payloads for extended timelines¹⁷. To achieve this goal for IA, we previously developed degradable polymers degrade over time in situ to release ITA at biologically-active concentrations³². This approach provides extended anti-inflammatory effects with a high loading capacity (50% of the material mass), and control over release kinetics (i.e., days-years) imparted through material chemistry approaches³³. Furthermore, incorporation of ITA into polymers enables the targeting of specific cells in a localized fashion.

The phagocytotic ability of innate immune cells, such as neutrophils, macrophages and dendritic cells (i.e. phagocytes), provides a potential therapeutic delivery approach to inflammatory cell targets¹⁸. In macrophages, phagocytosis is an intricate process enabling macrophages to engulf and degrade pathogens. The phagocytic ability of macrophages offers an opportunity for selective metabolite-based immunomodulatory therapy given an appropriate vehicle¹⁸, providing intracellular delivery tools targeted to inflammatory phagocytes. Polymeric microparticle drug delivery systems have been widely used toward this goal, leveraging tunable material properties including size and release kinetics³⁴ to deliver combinations of antigen, adjuvant, chemokines, and other immunomodulating molecules through phagocytosis and subsequent controlled release. The intracellular degradation rate of these microspheres can be regulated by altering the molecular weight and monomer composition of the copolymers that make up the microspheres^{35, 37}. Notably, numerous microparticle intracellular delivery technologies have been published with respect to PLGA delivery vehicles to achieve a variety of delivery applications³⁸⁻⁴⁷. For example, intracellular delivery of dexamethasone-loaded MPs can successfully downregulate inflammatory gene expression in human macrophages over several weeks, while minimizing systemic effects of prolonged glucocorticoid administration^{17, 48}. In dendritic cells, phagocytotic delivery of alpha-ketoglutarate polymer particle technologies have demonstrated sustained metabolite release, vaccine adjuvant delivery, and T-cell regulation in inflammatory models^{31, 49-56}.

### SUMMARY

In the present disclosure, the inventors were inspired by a small molecule, itaconate (ITA), that is evolutionarily preserved in the innate immunity⁵. Produced in activated macrophages through immune responsive gene 1 (IRG1)-itaconate axis⁶⁻⁸, recent findings have indicated the role of this molecule as an antibacterial and anti-inflammatory metabolite⁹.

In this disclosure, the present inventors harness the duality of ITA to both modulate infection and inflammation in a biomimetic strategy of polymer design. The present inventors hypothesized that such biomaterial could offer both improved material integration and reduced bacterial colonization at the cell-material interface. To do so, the present inventors present the scalable incorporation of ITA into a family of polyester material backbones and utilize hydrolytically driven degradation for the quantified sustained depot release from material surfaces. With quantified release from multiple material formulations, the present inventors demonstrate the ability to attenuate bacterial growth and macrophage inflammation in the local environment of ITA polymer materials in vitro and in vivo.

In this disclosure, the present inventors also introduce a robust and scalable method for synthesizing ITA-based polyester microparticles (ITA-MPs) designed for controlled intracellular degradation and release of ITA. ITA-MPs effectively modulate macrophage function, mimicking the immunoregulatory effects and metabolic rewiring effects of endogenous ITA. Given the challenge of delivering ITA systemically due to its limited potency and uptake, this platform offers a promising alternative for localized immune modulation.

Thus, in an example embodiment, there is disclosed herein a particle comprising a polyester biomaterial comprising diol monomers and itaconate, wherein the diol monomers comprise at least 1,12-dodecanediol.

In some example embodiments, the diol monomers further comprise 1,6-hexanediol, 1,8-octanediol, 1,10-decanediol, or a combination thereof.

In some example embodiments, the particle is formulated for the treatment of at least one of an infection or an inflammation, or a condition resulting directly from an infection or an inflammation, in a subject.

In some example embodiments, the particle has a particle size ranging from about 25 nm to about 100 μm.

In some example embodiments, the particle is formed by homogenization of a suspension or dispersion, spraying, microfluidic manipulation, vapor deposition, condensation, emulsion, coacervation, laser lysis, milling, fracture, seeding, lithography, or sol-gel methods.

In some example embodiments, the polyester biomaterial forms part of a coating for a polymer surface or a metal surface, and the polymer surface or the metal surface is a surface of a medical implantable device or a medical instrumentation device.

In some example embodiments, the polyester biomaterial is characterized by hydrolytic degradability.

In some example embodiments, the hydrolytic degradation of the polyester biomaterial causes release of therapeutic degradation products, and the degradation products comprise itaconate, an isomer of itaconate, or a combination thereof.

In some example embodiments, the degradation products further comprise itaconate bonded with at least one of the diol monomers, an isomer of itaconate bonded with at least one of the diol monomers, or a combination thereof.

In some example embodiments, the polyester biomaterial is formed by polycondensation of the diol monomers with the itaconate monomers in the presence of a radical inhibitor.

In some example embodiments, the polyester biomaterial is formed by polycondensation in a temperature range from about 120° C. to about 130° C. at atmospheric pressure.

In some example embodiments, the polyester biomaterial is formed by additional polycondensation at vacuum pressure.

In some example embodiments, the itaconate monomers comprise methylated itaconate.

In some example embodiments, the polyester biomaterial further comprises second carboxylate monomers.

In some example embodiments, the polyester biomaterial is formed by forming a polyester backbone including the diol monomers and the second carboxylate monomers, and reacting the polyester backbone with the itaconate monomers.

In some example embodiments, the polyester biomaterial is formed at atmospheric pressure at about 120° C.

In some example embodiments, the second carboxylate monomers comprise citrate.

In some example embodiments, there is herein disclosed a method of fabricating the polyester biomaterial, the method comprising:
forming a polyester backbone including the diol monomers; and
reacting the polyester backbone with the itaconate monomers.

In an example embodiment, there is herein disclosed aa method of treating at least one of an infection or an inflammation, or a condition resulting directly from an infection or an inflammation, in a subject, the method comprising:
providing a polyester biomaterial comprising diol monomers and at least first carboxylate monomers, wherein the first carboxylate monomers are itaconate and the diol monomers comprise at least 1,12-dodecanediol, and wherein the polyester biomaterial is in the form of a particle; and
administering the polyester biomaterial to the subject.

In some example embodiments, the diol monomers further comprise 1,6-hexanediol, 1,8-octanediol, 1,10-decanediol, or a combination thereof.

In some example embodiments, the method further comprises the step of encapsulating an active molecule using the particle, before the administering step.

In some example embodiments, the administering step comprises topical administration, enteral administration, or by subcutaneous, intradermal, intramuscular, intraocular, intraarticular, or intravascular injection.

In some example embodiments, the polyester biomaterial is provided as part of a scaffold before the administering step.

In some example embodiments, the scaffold is for a tissue patch.

### BRIEF DESCRIPTION OF THE DRAWINGS

Embodiments will now be described, by way of example only, with reference to the drawings, in which:

FIGS. 1A and 1B show development of bioactive polyester materials that harness the power of itaconate innate immunity through small molecule degradation release. FIG. 1A shows general synthesis scheme for an ITA containing polyester using dimethyl itaconate with alcohols under with a radical inhibitor and tin based catalyst and FIG. 1B shows images of synthesized ITA polyesters with (I) 1,6-hexanediol (II) 1,8-octanediol (OD), or (III) 1,10-decanediol (DD).

FIGS. 2A to 2L show characterization of itaconate containing polyester materials with demonstrated molecular release, in which: 2A to 2B shows characterization of polymer purity and structure. Representative (A)¹HNMR and (B) FTIR spectra for ITA+DD, ITA+OD, ITA+HD and SUC+OD polyester, 2C to 2E demonstrate hydrolytic release of ITA from material backbones. Integrated peak area of mass spectrometry assessment for ITA presence in hydrolytic degradation supernatant extracts for (2C) ITA+HD, (20) ITA+OD, and (2E) ITA+DD. Data is mean±SD, n=4. One way ANOVA followed by pairwise comparison with Tukey's multiple comparisons test, 2F to 2I demonstrate ITA containing material coating onto common medical devices surfaces. 2F shows ITA+HD was visualized with solubilized Sudan Red, and the (2G) ITA+HD and (2H) similarly prepared ITA+OD were coated onto PVC tubing (ID: ⅛″, OD:¼″ (2G), OD: 3/16″ (2H)). (2I) ITA+DD was solvent cast onto the surface of medical grade titanium alloy as a solid (room temperature), becoming gel form at physiologically relevant temperature (37° C.). Scale: 1 mm per notch, P indicates polymer coating. 2J to 2L show culture of dermal fibroblasts on ITA+OD presents similar cellular behaviour to polystyrene controls. 2J shows quantified percent of viable dermal fibroblasts 2 days post seeding. Data is mean±SD, n=6. Student t-test. Representative fluorescent images of dermal fibroblasts attachment on (2K) ITA+OD polymer-coated well (2 d post-seeding) and (2L) tissue culture polystyrene control 2 d post-seeding. Green: CFSA-SE (Live Cells), Red: PI (dead cells), Blue: DAPI (cell nuclei). Scale bars: 150 μm. Statistical significance is indicated as *p<0.05 ** p<0.01, *** p<0.001, **** p<0.0001, ns=non-significant.

FIG. 3A to 3G show degradation behavior of itaconate containing materials in which: 3A shows representative peak identification of ITA, HD, OD, and DD in mass spectroscopy (m/z) spectra, 3B, 3C and 3D show integrated peak area for (3B) HD, (3C) OD, and (3D) DD in hydrolytic degradation supernatant extracts for ITA+HD, ITA+OD, and ITA+DD respectively. Data is mean±SD, n=4. One way ANOVA followed by pairwise comparison with Tukey's multiple comparisons test. Statistical significance is indicated as *p<0.05 ** p<0.01, *** p<0.001, **** p<0.0001. 3E shows hydrolytic degradation of ITA containing polyesters in DPBS over 28 days, 3F and 3G show accelerated degradation of: 3F ITA+OD under basic conditions (1M) and 3G ITA+DD in the presence of lipase.

FIGS. 4A to 4G show itaconic acid (ITA) based polyesters inhibit bacterial growth, in which 4A shows soluble ITA inhibition of E. coli growth below 1 mM concentration with an acetate carbon source, 4B to 4F show liquid culture growth inhibition of E. coli cultured with ITA polymer materials (* p<0.05). 4B shows ITA+HD, ITA+OD, and ITA+DD growth inhibition on acetate in comparison to a polymer-free growth control, whereas 4C shows growth on a glucose source indicated no appreciable inhibition, 4D shows ITA+DD performed similar inhibition to silver nanoparticles and 4E provided improved inhibition comparted to a PLLA degradable polyester analogue (acetate source), 4F shows inhibition results remained consistent across multiple synthesis batches of ITA+HD, suggesting the reproducibility of antimicrobial efficacy. Data is mean±SD, n≥3. One way ANOVA followed by pairwise comparison with Tukey's multiple comparisons test. Statistical significance is indicated as *p<0.05. 4G presents ITA containing polyester gel prevent infection-based lethality. Mice injected with ITA+OD, silicone or saline (n=10) three days prior to E. coli bacterial loading present differential survival characteristics. Saline+saline control (n=5) received saline three days prior and saline at time of infection loading.

FIG. 5A to 5H show itaconate polymers present growth specific E. coli inhibition, in which: 5A to 5D show soluble itaconate inhibition under different media conditions. Solubilized itaconate (up to 50 mM) did not impact growth on 5A minimal M9 media with a glucose carbon source or 5B LB Broth. 5C to 5D show dimethyl itaconate exhibited more pronounced inhibition, demonstrating inhibition with 5C LB broth (above 7.5 mM) and 5D inhibition comparable to solubilized itaconate under acetate growth conditions in M9 media. 5E shows an image of experimental setup used for inhibition assessment. 5F shows a culture of ITA containing materials in LB broth did not exhibit appreciable growth inhibition. 5G shows inhibition was demonstrated across a range of ITA+DD synthesis times (x+y hr indicates x: time at 1 atm, y: time at vacuum pressure), with more pronounced inhibition with lower reaction time (acetate growth substrate). 5H shows inhibition was not observed with substitution of itaconate with adipate in the synthesis method. Data is mean±SD, n≥3. One way ANOVA followed by pairwise comparison with Tukey's multiple comparisons test. Statistical significance is indicated as *p<0.05.

FIGS. 6A to 6M show itaconate containing materials modulate macrophage cytokine inflammatory response, in which: 6A shows a schematic of experimental protocol for the generation and assessment of polymer immunomodulation behaviour with murine bone marrow derived macrophages in vitro, 6B to 6M shows inflammatory cytokine quantification for key markers in innate immunity. Culture with ITA+DD (6B to 6G) and ITA+OD (6H to 6M) material with soluble DMI comparison demonstrated a downregulation of IL-1β (6B, 6H), IL-6 (6C, 6I), CCL2 (60, 6J), IL-12 p70 (6F, 6L), IFNβ (6G), and IL-10 (6M) with maintenance of TNFα expression (6E, 6K) when compared to PLGA inserts and insert free controls. + indicates stimulation with LPS (100 ng/mL), ++ indicates stimulation with LPS (100 ng/mL) and IFNγ (50 ng/mL). Data is mean±SD, n4. One way ANOVA followed by pairwise comparison with Tukey's multiple comparisons test. Statistical significance is indicated as *p<0.05 ** p<0.01, *** p<0.001, **** p<0.0001.

FIGS. 7A to 7I show in vitro culture of itaconate materials with bone marrow derived macrophages presents repeatable immune modulation, in which: 7A shows images of experimental setup, polymer was coated on 18 mm glass coverslips and submerged in media wells (6 well culture plate) using transwell inserts, 7B shows the quantification of lactate dehydrogenase release from BMDM cultured with the conditions assessed in inflammatory experiments, 7C shows bright field images of stimulated BMDM cells (LPS: 100 ng/mL) cultured with ITA+DD compared to a PLGA and insert free (Blank) control. Scale bar: 100 μm. 7D to 7I show inflammatory cytokine quantification for key markers in innate immunity when pretreated with itaconate containing materials (12 hr) followed by stimulation with LPS (100 ng/mL) compared to PLGA inserts and insert free controls. 7D shows pretreatment of BMDM with ITA+DD presents a downregulation of IL-10. 7E to 7I show pretreatment of ITA+HD presents a trend of downregulation in IL-1β (7E), CCL2 (7H), and IL-10 (7I), without observed trend in IL-6 (7G) or TNFα (7F) expression. + indicates stimulation with LPS (100 ng/mL). Data is mean±SD, n≥3. One way ANOVA followed by pairwise comparison with Tukey's multiple comparisons test. Statistical significance is indicated as *p<0.05 ** p<0.01, *** p<0.001, **** p<0.0001.

FIGS. 8A to 8E show itaconate containing materials induce reduction in phenotypic inflammation, in which: 8A shows a representative histogram of fluorescence indicating expression of nitrate oxide synthase (NOS2) in response to stimulation (LPS or LPS/IFNγ) in cells pretreated with ITA+HD, ITA+OD, ITA+DD, soluble DMI, PLGA or insert free controls with an unstimulated reference, 8B to 8C show quantified median fluorescence intensity for NOS2 expression for cells pretreated (12 hr) with ITA+DD materials and stimulated with (8B) LPS (100 ng/mL) or (8C) LPS (100 ng/mL) and IFNγ (50 ng/ml) for 12 (hr), 8D to 8E show supernatant nitrite content for (8D) gel materials (ITA+HD, ITA+OD) stimulated with LPS and (8E) ITA+DD stimulated with LPS (left) or LPS and IFNγ (right). + indicates stimulation with LPS (100 ng/mL), ++ indicates stimulation with LPS (100 ng/mL) and IFNγ (50 ng/mL). Data is (8B-8C) median ±SD, n≥3, (8D to 8E) mean±SD, n≥4. One way ANOVA followed by pairwise comparison with Tukey's multiple comparisons test. Statistical significance is indicated as *p<0.05 ** p<0.01, *** p<0.001, **** p<0.0001.

FIGS. 9A to 9J show injectable itaconate gel material reduces biomaterial host response in a peritoneal infiltration model, in which: 9A shows an experimental schematic for peritoneal biomaterial associated immune cell infiltration model, 9B to 9J show, using flow cytometry assessment, quantified immune cell infiltration into the peritoneum following injection of ITA+OD (50 μL), hydroxy terminated polydimethylsiloxane (50 μL), or saline (Sham), with a comparable untreated group (Blank), 9B shows representative flow cytometry plots for ITA+OD (left), silicone (centre), and saline sham (right) ten days post injection, for which quantified cell populations were determined (9E to 9F) three and (9G to 9J) ten days post injection; at each timepoint, we quantified (9C, 9G) neutrophil, (9D, 9H) early monocyte, (9E, 9I) eosinophil and (9F, 9J) resident macrophage populations. Data is mean±SD, n≥3. One way ANOVA followed by pairwise comparison with Tukey's multiple comparisons test. Statistical significance is indicated as *p<0.05 ** p<0.01, *** p<0.001, **** p<0.0001.

FIG. 10 shows gating strategy for single cell assessment of peritoneal immune cell infiltration.

FIGS. 11A to 11J show injectable itaconate gel exhibits a dose dependent biomaterial host response in a peritoneal infiltration model, in which: 11A to 11J show quantified assessment of single cell distribution, (11A to 11E) three and (11F to 11J) ten days post injection of ITA+OD or hydroxy terminated polydimethylsiloxane (High Dose: 150 μL, Low Dose:50 μL), compared to saline sham (150μL) and untreated controls (Blank), 11A, 11F show total live cell number post injection, 11B to 11E, 11G to 11J show quantified infiltration of (11A, 11E) neutrophil, (11B, 11F) early monocyte, (11C, 11G) eosinophil and (11D, 11H) resident macrophage populations with injection of a 150 μL dosage of biomaterial groups. Data is mean±SD, n≥3. One way ANOVA followed by pairwise comparison with Tukey's multiple comparisons test. Statistical significance is indicated as *p<0.05 ** p<0.01, *** p<0.001, **** p<0.0001.

FIG. 12 shows general polycondensation synthesis scheme of PICO polymer materials.

FIGS. 13A to 13D show polymer composition and differential conversion, in which: 13A shows representative ¹H-NMR spectra with labelled peaks according to molecules in the polymer backbone and monomer specific end groups, 13B shows conversion of OD into a TEC-OD (1:1 mol:mol) material over course of a 6 hour reaction, 13C to 13D show ITA incorporation into polymer materials in which 13C shows percentage of incorporated groups that are converted into ester bond, and 13D shows yield of expected ITA incorporation relative to OD basis.

FIGS. 14A to 14D show PICO materials prepolymer composition, in which: 14A to 14C show representative ¹H NMR spectrum PICO monomers: (14A) triethyl citrate (14B) dimethyl itaconate, and (14C) 1,8-octanediol, and 14D shows representative FTIR spectra of PICO gel prepolymer.

FIGS. 15A to 15D show gelation time is tunable according to polymer composition, in which: 15A to 15C shows time sweep measurement of storage (G′) (●) and loss (G″) (▪) modulus with exposure to UV light (illuminated at t=100 s, indicated by the vertical dashed line) to present gelation characteristics. PICO with (15A) high, (15B) low, and (15C) moderate DMI monomer feed (ITA content) presented a modulation in gelation with differentiating polymerization time, 15D shows summarized time to gelation post UV light illumination, defined as the crossover point of storage and loss modulus. No evident gelation was observed in Polymers 4, 5, and 8.

FIGS. 16A to 16E show mechanical property characteristics of crosslinked polymer samples in which: 16A shows representative stress-strain curves of polymers with differing feed ratios, 16B to 16E shows comparative material properties of polymer groups exposed to 800 mJ of UV energy including (16B) Young's Modulus (16C) swelling in water (16D) elongation at break, and (16E) ultimate tensile strength (n=3). Statistics from two-way ANOVA are indicated in the tables (ns: non-significant difference). Significant differences between samples within the same feed ratio are defined as *p<0.05, **p<0.001, ***p<0.0001.

FIG. 17A to 17D show cyclic loading properties of PICO elastomers, in which: 17A shows representative cyclic tensile loading for polymer 10 over the first 10 cycles, 17B to 17D show stress-strain curves for cyclic tensile test at increasing cycles up to 1500 cycles for crosslinked samples of Polymer 2 (17B), 6 (17C), and 10 (17D).

FIGS. 18A and 18B show mechanical property characteristics of crosslinked polymer samples with variation in time, in which: 18A shows swelling of Polymer 10 crosslinked samples in water over three days. Assessment with One Way ANOVA indicated no significant differences. 18B shows modulation of Polymer 9 elasticity according to exposure energy.

FIGS. 19A to 19D show crosslinked polymer samples present differential degradation, in which: 19A to 19C show accelerated (0.25M NaOH) degradation of UV crosslinked PICO materials with (19A) high (19B) low and (19C) moderate DMI monomer feed (ITA content). 19D shows a comparison of mass loss of material groups with 48 hours of degradation. Two Way ANOVA presents significance of reaction time and ITA content on hydrolytic degradation of crosslinked materials. Statistics from two-way ANOVA are indicated in the tables (ns: non-significant difference). Data are presented as mean±SD, n=4. Significant differences between samples within the same monomeric feed ratio are defined as *p<0.05, **p<0.001, ***p<0.0001.

FIGS. 20A-C show PICO polymer scaffold as cardiac patches in which: FIG. 20A shows a schematic representation of PICO scaffolds fabricated using microfabrication technology, FIG. 20B shows cardiac tissue cultured on PICO scaffolds affixed on holders in a tissue culture dish (Scale bar: 1 cm), FIG. 20C shows cardiac tissue compaction around PICO scaffolding in hydrogel ECM (Scale Bar: 500 μm), FIGS. 20D-20G shows fluorescence images of live (CFDA-SE: green) and dead (PI: red) rat neonatal CMs on PICO scaffolds. Low (FIG. 20D, FIG. 20E) and high (FIG. 20F, FIG. 20G) density constructs were visualized. Cell nuclei were also stained with DAPI (blue), and the scaffold exhibits autofluorescence in this channel. (Scale bars: FIGS. 20D and 20F=200 μm, FIGS. 20E and 20G=100 μm), FIG. 20H shows quantified cell death of cardiac fibroblasts cultured for 24 hours in dilutions of conditioned culture media treated with Polymers 3, 6, and 10 compared to the untreated culture media (Control) and a positive control media containing 10% Triton-X (Positive). Data are presented as mean±SD, n≥4 *p<0.05, One-way ANOVA followed by Dunnett's multiple comparisons test.

FIGS. 21A-21D show fluorescence images of high CM density PICO scaffold construct. (FIG. 21A) Composite image of live (CFDA-SE: green) and dead (PI: red) stain of rat neonatal CMs. Cell nuclei were also stained with DAPI (blue), and the scaffold exhibits autofluorescence in this channel. (FIGS. 21B-D) Single channel fluorescence images of the (FIG. 21B) green (CFDA), (FIG. 21C) red (PI), (FIG. 21D) and blue (DAPI) channels. Scale bars: 100 μm.

FIGS. 22A to 22F show mechanism and development of ITA-MPs comprising bioactive polyester materials that harness the power of itaconate innate immunity through small molecule degradation release. FIG. 22A is a schematic showing the mechanism of ITA-MP as a potential therapeutic strategy to target pro-inflammatory macrophage activity. FIG. 22B shows a general synthesis scheme for an ITA using dimethyl itaconate and 1,12-dodecanediol (DoD) with a radical inhibitor and tin based catalyst. FIG. 22C demonstrates characterization of polymer structure in a ¹HNMR spectrum for ITA-DoD polyester, wherein subsequent peak identification confirmed the formation of a linear polymer material with maintenance of the pendant unsaturated carbonyl on ITA. FIG. 22D shows a ¹HNMR spectrum of bulk ITA-DoD polymer demonstrating spontaneous isomerization of backbone ITA residues to mesaconate (represented by the peak ‘i’), an isomer of ITA. FIG. 22F demonstrates the increase over a 48 h time period of ITA release in polymer supernatant treated with deionized distilled water. FIG. 22E shows images of spherical polymer microparticles from poly(ITA-DoD) material.

FIGS. 23A to 23C demonstrate particle phagocytosis, cell viability, and apoptotic behaviour at 24 h of treatment following live cell imaging of macrophages exposed to ITA-based polymeric microparticles at varying concentrations (0.5, 0.25, 0.1, 0.05, 0.025, 0.01, and 0.001 mg·10⁶ cells−1). FIG. 23A shows cell death at various concentrations of ITA-MP and the LPS media control, measured using quantitative cell death staining. FIG. 23B shows cell counts achieved through DAPI nuclear labeling and serial quantification normalized to initial values. FIG. 23C shows representative endpoint images at concentrations of 0, 0.5, 0.1, 0.01 mg·10⁶ cells⁻¹. FIG. 23D shows phagocytosis after 24 hours of treatment with 0.1 mg·10⁶ cells⁻¹, 99.9% of BMDMs. FIGS. 24A to 24K show macrophage ITA-MP uptake quantification and characterization of resultant cell phenotype. Quantification of inflammatory cytokine expression indicated unchanged TNF-α MFI in ITA-MP-treated BMDMs compared to the LPS media control, which diverged from the results observed with 4OI and soluble ITA which both demonstrated a significant reduction in TNF-α expression (FIG. 24A). ITA-MP treated BMDM demonstrated a significant upregulation of IL-6 expression (FIG. 24B) compared to a stimulated untreated control, consistent with the effect observed from soluble ITA. This trend was similar in the MFI of IL-12p40 (FIG. 24C), and MHC-II (FIG. 24D), which were significantly upregulated in ITA-MP treated cells. Although an average increase in MFI was observed in these markers, up-regulation and distribution of expression was observed was noted to be dependent on particle loading (FIG. 24E-H). Cells positive for expression of IL6, IL12p40, and MHCII were isolated, and then subsequently gated macrophages based on absolute internalization of ITA particles determined by PE intensity (rhodamine signal from particles) vs side scatter signal (FIG. 24E). Quantification of MFI in the high and low group, contrasted to particle free controls with and without LPS stimulation provided insight into the role of particle loading on relative expression. Expression of IL-6 (FIGS. 24F,I), IL12p40 (FIG. 24G,J), and MHCII (FIG. 24H,K) were significantly reduced in the high ITA-MP group when compared to low ITA-MP loaded cells. Quantified MFI of expression in the high ITA-MP group was notably reduced in IL12p40 and MHCII expression, which were significantly reduced compared to particle free LPS stimulated controls.

FIGS. 25A to 25E show characterization of metabolic utilization using the SCENITH approach to measure changes to metabolic organization associated with ITA-MP uptake. FIG. 25A shows a protein synthesis scheme and corresponding exogenous puromycin incorporation in the presence of the metabolic toxins 2-deoxyglucose (2DG, a glycolytic inhibitor) or oligomycin (oxidative electron transport chain inhibitor). ITA-MP-treated BMDM displayed a significantly higher puromycin mean fluorescence intensity (MFI) under oligomycin treatment compared to LPS-stimulated and unstimulated controls, indicating reduced dependence on oxidative phosphorylation (FIGS. 25B-C). Correspondingly, 2DG decreased puromycin MFI significantly in the same treatment, further indicating primarily glycolytic dependence. Despite these metabolic shifts, gene expression analysis of key glycolytic markers (Glut-1 and HK-1) showed a slight upregulation of Glut-1 and no significant differences in HK-1 expression between BMDM treated with ITA-MP and LPS media controls (FIG. 25D-E).

FIG. 26 shows representative flow cytometry plots for BMDM treated with ITA-MPs at concentrations of 5 mmol L⁻¹ soluble itaconate (ITA), and 0.125 mmoI^{L-1} 4OI with 100 ng mL⁻¹ of LPS.

FIG. 27 shows representative flow cytometry plots for live samples profiled in single-cell format for metabolic function using methods adapted from the SCENITH workflow.

### DETAILED DESCRIPTION OF THE EMBODIMENTS

Without limitation, the majority of the systems described herein are directed to a particle comprising polyester biomaterial containing itaconate and at least 1,12-dodecanediol, and a method of treating at least one of an infection or an inflammation, or a condition resulting directly therefrom, by administering the polyester biomaterial containing itaconate and at least 1,12-dodecanediol in the form of a particle to a subject. As required, embodiments of the present invention are disclosed herein. However, the disclosed embodiments are merely exemplary, and it should be understood that the invention may be embodied in many various and alternative forms.

The accompanying figures, which are not necessarily drawn to scale, and which are incorporated into and form a part of the instant specification, illustrate several aspects and embodiments of the present disclosure and, together with the description therein, serve to explain the principles of the simulation apparatus. The drawings are provided only for the purpose of illustrating select embodiments of the apparatus and as an aid to understanding and are not to be construed as a definition of the limits of the present disclosure.

As used herein, the term “about”, when used in conjunction with ranges of dimensions, temperatures or other physical properties or characteristics is meant to cover slight variations that may exist in the upper and lower limits of the ranges of dimensions so as to not exclude embodiments where on average most of the dimensions are satisfied but where statistically dimensions may exist outside this region. For example, in embodiments of the present invention dimensions, composition, and characteristics of components of a neck simulator may be given but it will be understood that these are not meant to be limiting.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

Although successes are notable, the use of synthetic biomaterials have been limited by poor integration, fibrosis, bacterial colonization of implants and limited biocompatibility. Through a biomimetic approach, the present inventors have developed a library of polyester materials that incorporate a powerhouse innate mammalian cell immunity, itaconic acid (ITA), into polymer backbones for establishment of inherent antibacterial and anti-inflammatory characteristics. Using a one pot polycondensation reaction, the inventors optimized an adaptable synthesis method to combine ITA with different length alcohol monomers, yielding materials of high purity (1 HNMR, FTIR) in gel (1,6-hexanediol, 1,8-octanediol), and solid form (1,10-decanediol). These constructs demonstrated quantifiable ITA release in a hydrolytic environment.

Multiple ITA containing material combinations demonstrated a decrease in Escherichia coli growth (p<0.05) when compared to a polystyrene and poly(L-lactic acid) (PLLA) groups, with comparable inhibition to commonly utilized silver nanoparticles. Pre-treatment of murine bone marrow derived macrophages (BMDMs) with ITA containing materials prior to pro-inflammatory stimulation (LPS, LPS/IFNγ) presented a significant down-regulation in a number of pro-inflammatory cytokines (IL-1β, IL-6, IL-10, IL-12p70, CCL2, IFNβ) and phenotypic nitric oxide production (NOS2 expression, nitrile secretion) when compared to poly(lactic-co-glycolic acid) (PLGA), suggesting release mediated anti-inflammatory characteristics.

Specificity of material cell functionality was verified by demonstrated non-toxic behaviour with human dermal fibroblasts. Upon peritoneal material injection, ITA containing gel material presented reduced biomaterial associated inflammation (neutrophils, monocytes, eosinophils; p<0.05) when compared to silicone ten days post-implant. The inventors have demonstrated a novel biomimetic approach where the inventors harness the advantages of the innate immunity, using a biomaterial design to incorporate bioactivity into polymer backbones, achieving localized antibacterial and anti-inflammatory material properties. These outcomes indicate the potential of ITA based material design as a platform of active material microenvironments with dual functionality for improvement of material adoption.

However, the use of ITA in immunomodulatory therapies has faced limitations due to inherent challenges in achieving controlled and sustained delivery. Microparticle (MP)-based delivery strategies are a promising approach for intracellular delivery of small molecule metabolites through targeting of macrophage phagocytosis and subsequent intracellular polymer degradation-based delivery. Toward the goal of intracellular delivery of ITA, degradable polyester polymers (poly(itaconate-co-dodecanediol)) based ITA polymer microparticles (ITA-MPs) were generated using an emulsion method, forming micron-scale (˜2 μm) degradable microspheres. ITA-MPs were characterized with respect to their material properties and ITA release to inform particle fabrication.

Treatment of murine bone marrow-derived macrophages with an optimized particle concentration of 0.1 mg/million cells confirmed internalization and low levels of cytotoxicity of ITA-MP. Flow cytometry demonstrated ITA-MP-specific regulation of ITA-sensitive inflammatory targets, similar to soluble ITA controls with significantly reduced ITA release. Metabolic analyses demonstrated that ITA-MP internalization inhibited oxidative metabolism and induced glycolytic reliance, consistent with the established mechanism of ITA-associated inhibition of succinate dehydrogenase. This development of ITA-based polymer microparticles provides a basis for additional innovative metabolite-based microparticle drug delivery systems for the treatment of inflammatory disease.

### Results

### Synthesis And Molecular Release From Itaconate Containing Polyesters

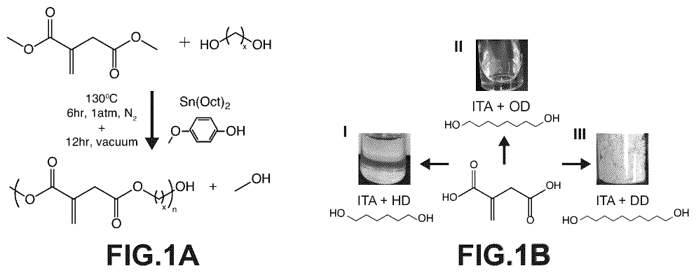

To create a biomaterial that can mimic some functions of the innate immunity and facilitate long-term delivery of ITA to the local cell microenvironment, we used polyester polymer synthesis techniques to incorporate the bioactive molecule into a hydrolytically degradable material. Given the reactivity of the pendant acrylate group on ITA, we employed a one-pot polycondensation with methylated carboxylate monomers (dimethyl itaconate, DMI) in the presence of a radical inhibitor (4-methoxyphenol, MEHQ) as descried previously⁶⁰ (FIG. 1A). Using this method, we have successfully synthesized new polyesters incorporating ITA into the polymer backbone, where we have coupled diols with ITA to generate long chain polyester materials. By varying the di-alcohol reacted with ITA, we have generated macroscopic gels, (poly[(itaconate)-co-(1,6-hexanediol)](ITA+HD) and poly[(itaconate)-co-(1,8-octanediol)](ITA+OD)), and solid ((poly[(itaconate)-co-(1,10-decanediol)](ITA+DD)) materials (FIG. 1B).

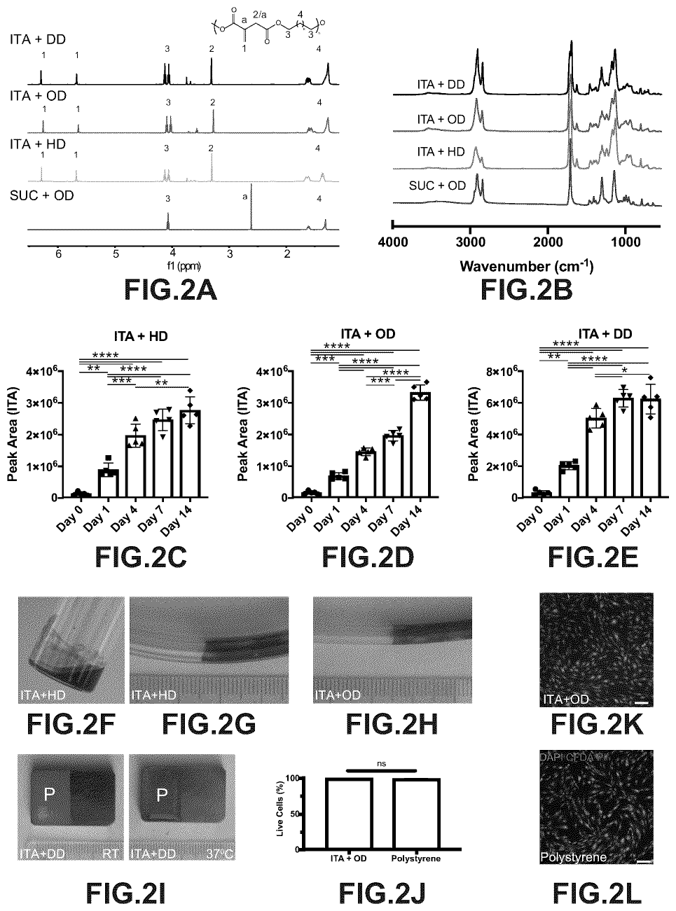

Chemical structure assessment using proton nuclear magnetic resonance spectroscopy (¹HNMR) (FIG. 2A) and Fourier transform infrared spectroscopy (FTIR) (FIG. 2B) confirms polymer backbone structure and the presence of the ITA acrylate content when compared to succinate containing materials. Succinate, which lacks the acrylate group, was chosen as a representative control to synthesis efficacy given its similarity in backbone structure (i.e. four carbon carboxylate). Maintenance of this acrylate group is essential for polymer degradability and bioactivity, and the success of our synthesis method at accomplishing this was further confirmed by homogenous material appearance and solubility (soluble in non-polar solvent including chloroform and ethyl acetate). ITA+DD materials were solid at room temperature with a melting point of 30-32° C., much lower than polymers containing succinate, which were solid in appearance with high melting temperatures (data not shown), suggesting the alkene group on ITA impacts polymer chain molecular organization.

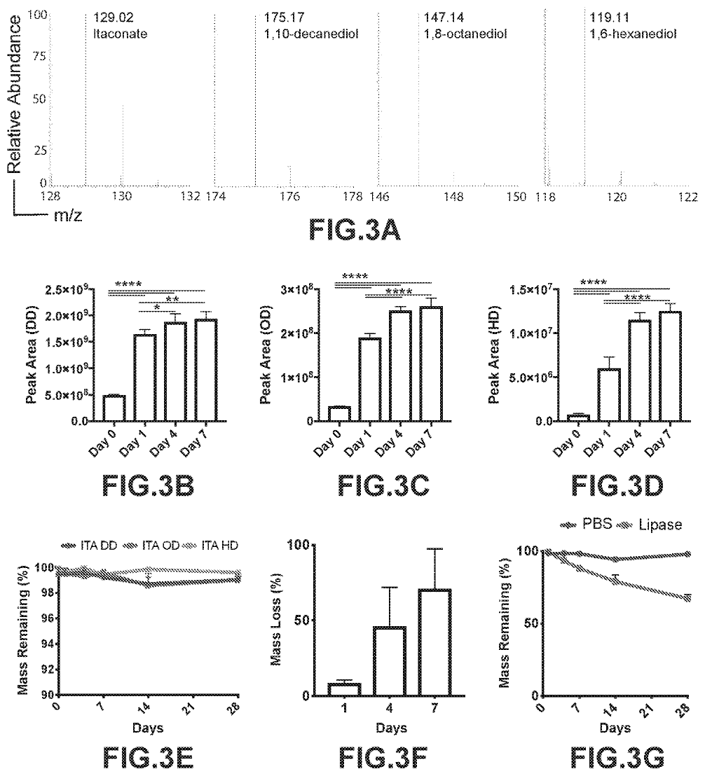

Hydrolytic degradation of ITA polyesters was evaluated to determine the release profile of ITA from material backbones. ITA was identified in the m/z spectrum of liquid chromatography-mass spectrometry (LC-MS) assessment of the degradation supernatant of ITA+HD, ITA+OD, and ITA+DD (FIG. 3A). Integration of peak area presents an increase in itaconate release over 14 days in all materials (FIG. 2C-E), with greater observed release in ITA+DD material (FIG. 2E). Corresponding diol release was increased over time proportionally to ITA, suggesting degradation release from the polymer backbones (FIG. 3B-D). Gross bulk mass loss was stable under hydrolytic conditions (1×PBS; <2% over 28 d) (FIG. 3E) but was accelerated under basic conditions (ITA+OD, 1M NaOH) (FIG. 3F) and in the presence of lipase (ITA+DD) (FIG. 3G), suggesting material susceptibility to hydrolytic and enzymatic degradation.

### Specificity and Application of Material Constructs

As described, the one-pot synthesis method employed here allows for incorporation of ITA with various alcohols for generation of different material properties. Herein, we focused on linear materials for simplicity of assessment of material functionality. In application, gel materials (mixed with Sudan Red for visualization; FIG. 2F) ITA+HD (FIG. 2G) and ITA+OD (FIG. 2H) can surface coat commonly used medical tubing. ITA+DD was solvent cast onto medical grade metal implants at room temperature, presenting stability for storage, and becomes a gel when heated to body temperature (37° C.) (FIG. 2I). The modulatory behavior of released ITA did not provide a demonstrated impact on cell viability of human dermal fibroblasts, after two days of culture, quantified by immunostaining (live:CFDA-SE, dead:PI; FIG. 2J). Representative images of cells seeded on ITA+OD (FIG. 2K) and stained for viability markers present no marked difference from tissue culture polystyrene controls (FIG. 2L). This suggests that ITA release is non-impacting on general mammalian cell behavior and supports the specificity of biomaterial activity on immune modulation and infection prevention in potential applications.

### Inhibitory Behavior of Itaconate Materials on Escherichia Coli Growth

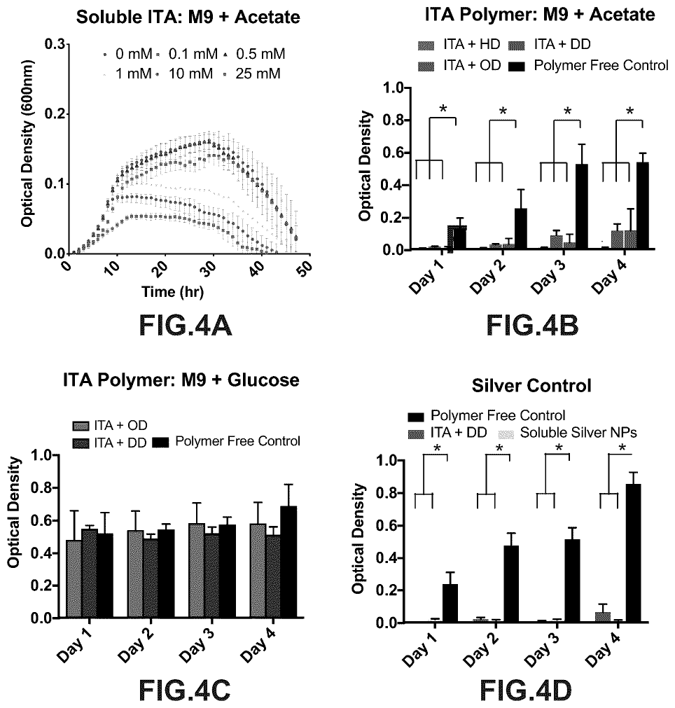

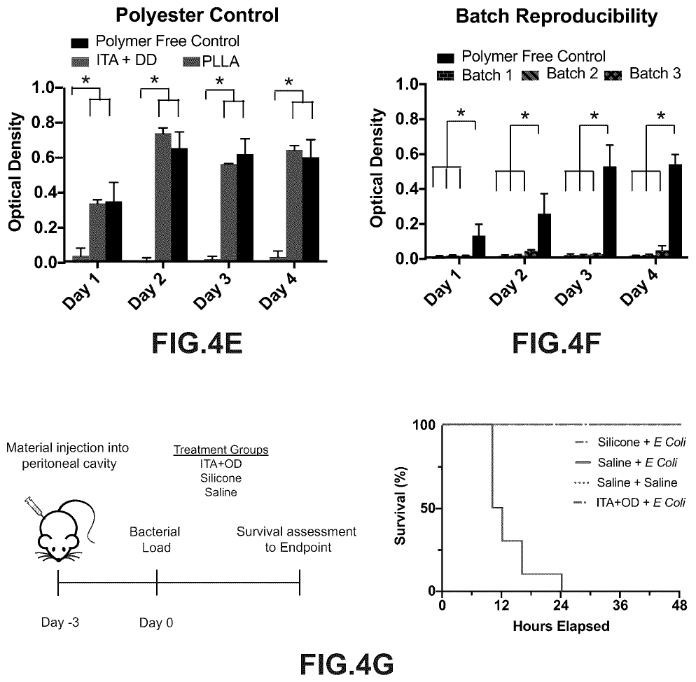

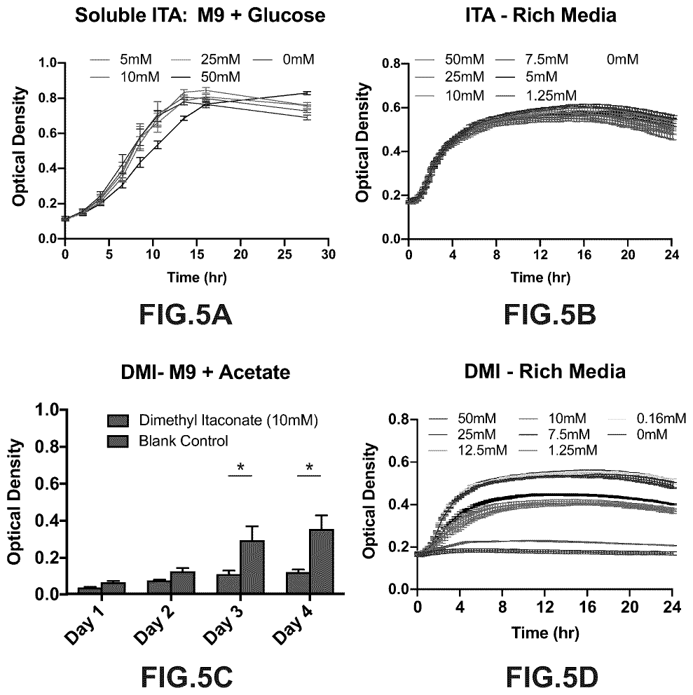

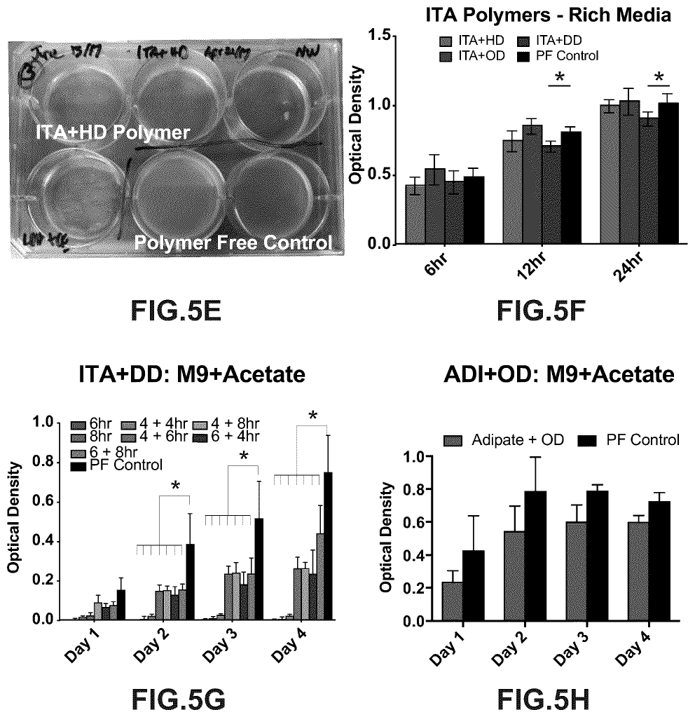

With the establishment of degradable ITA polyester materials, we assessed the translation of biomimetic antimicrobial efficacy of ITA containing polymers in vitro. It has been shown previously that ITA is a potent inhibitor of ICL, a key enzyme in the glyoxylate shunt that is necessary for bacterial growth on complex 2-carbon substrates (i.e. acetate)^{10, 11}. Therefore, we considered the benchmark for in vitro material efficacy to be specific growth inhibition in minimal media (M9) with an acetate carbon source (1% m/v). Culture of Escherichia coli with solubilized ITA under these conditions yielded growth inhibition as low as 1 mM concentration (measured by OD:600 nm) (FIG. 4A). This inhibition was not observed with a glucose carbon source (0.4% m/v) (FIG. 5A), or on rich media (LB broth) (FIG. 5B), consistent with previously observed inhibition specificity⁶¹. Culture with solubilized DMI, a polymer mimic (methylated ITA), presented similar effects on bacterial growth in complex conditions (FIG. 5C) with addition of some sensitivity (>7.5 mM) on rich media (FIG. 5D).

Assessment of growth inhibition from polymer degradation release was conducted in well plates with polymer films compared to polymer-free controls with optical density assessment (FIG. 5E). Here, ITA+HD, ITA+OD and ITA+DD demonstrated a profound bacterial growth reduction compared to the polymer-free controls (FIG. 4B). Culture of ITA+OD and ITA+DD in the media conditions with a glucose carbon source did not present any appreciable inhibition (FIG. 4C), and only slight inhibition for ITA+DD on LB broth (FIG. 5F) suggesting the specificity of inhibition to acetate conditions. Remarkably, when compared to commercially available silver nanoparticles, a current gold standard in antimicrobial materials, ITA+DD exhibited similar bacterial growth inhibition (no significance between silver and polymer groups) (FIG. 4D). In contrast, degradable PLLA did not exhibit bacterial growth inhibition, nor did poly (adipate-co-octanediol) (ADI+OD), synthesized using the same approach as with ITA polymers (FIG. 5G), suggesting the itaconate content in ITA+DD polymers was responsible for inhibition (FIG. 4E). These results were reproducible across three independent synthesis batches of ITA+HD (FIG. 4F). With ITA+DD materials, the inhibition was tunable according to polymer reaction times, suggesting the potential for material optimization for the desired application (FIG. 5H).

Translation of in vitro inhibitory characteristics was assessed with an in vivo survival model. Mice injected with ITA+OD or silicone (150 μL, intraperitoneally) three days prior to infection loading (1×10⁹ CFU E. coli per mouse), presented with complete survival (48 hr, n=10) when compared to those injected with a saline sham (FIG. 4G). No evident differences were observed in biomaterial treated groups.

### Anti-Inflammatory Characteristics

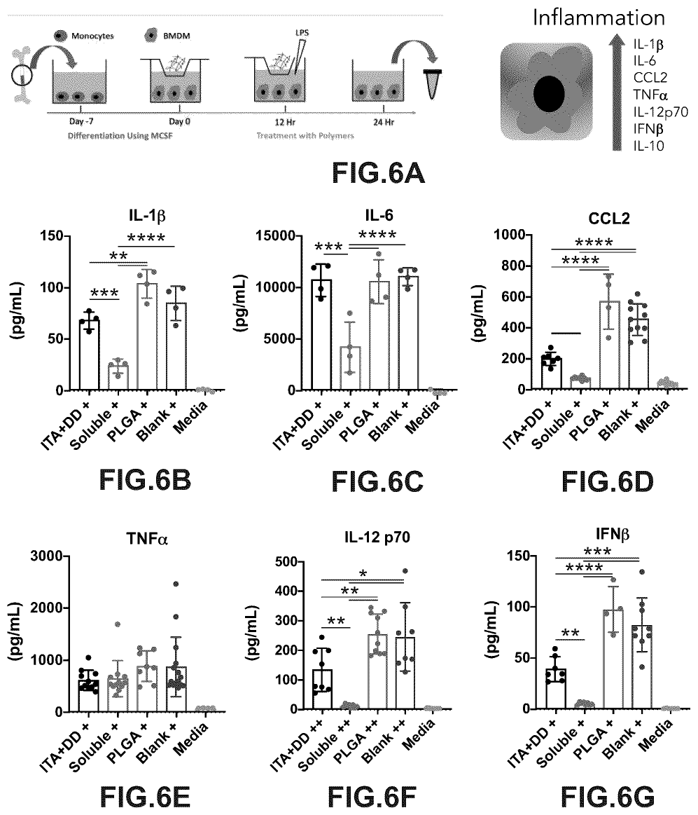

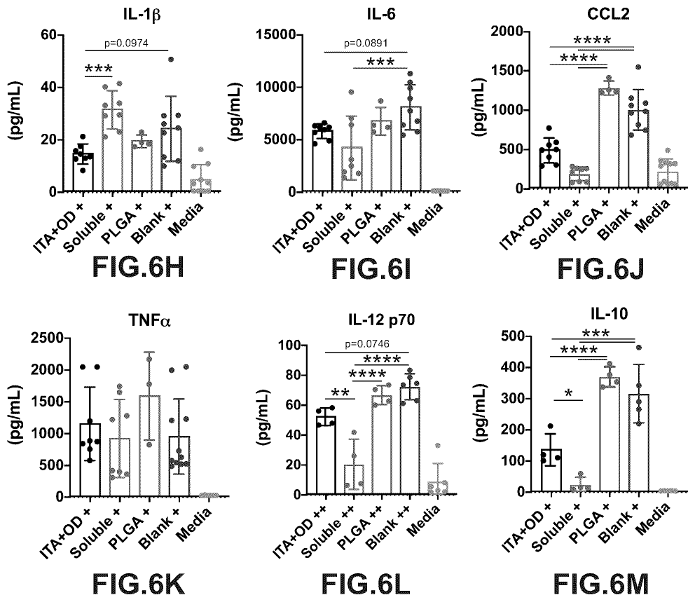

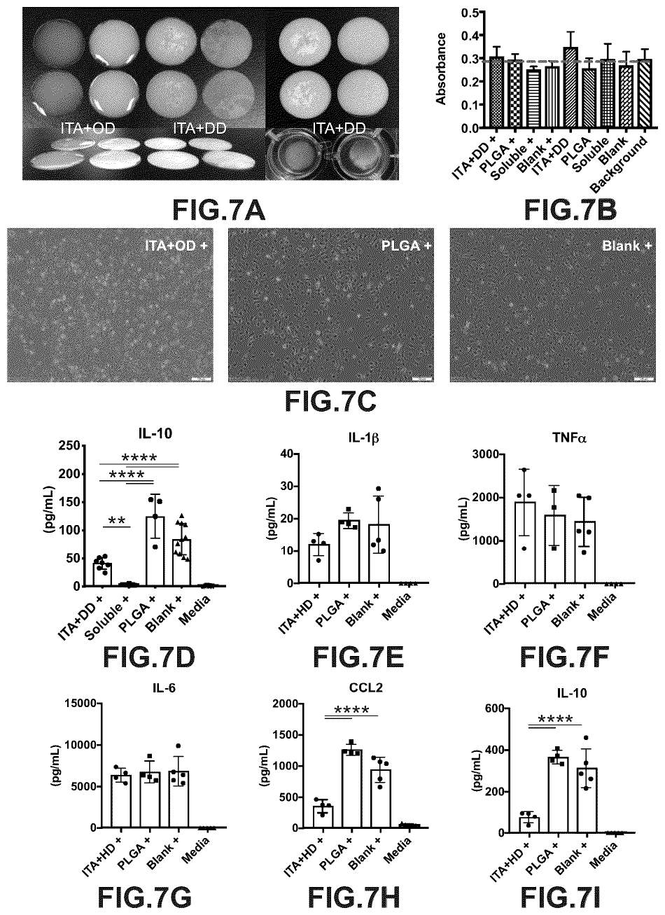

To understand the anti-inflammatory properties of ITA containing polyesters, we differentiated primary murine bone marrow derived macrophages (BMDM), and assessed the impact of polymer treatment on their inflammatory response (FIG. 6A). Given our interest in the degradation release of ITA into the local environment, we coated materials on glass coverslips (18 mm) and used transwell inserts to facilitate ITA release into the culture media without direct cellular contact (FIG. 7A). Similar to previous studies with ITA, we maintained quantified cell viability (FIG. 7B) and monolayer phenotype (FIG. 7C) with treatment and stimulation. Cells were pretreated for 12 hr with ITA containing materials, solubilized DMI (0.125 uM), PLGA, or media (insert free), then stimulated with LPS (100 ng/mL, noted as +) or LPS (100 ng/mL) and IFNγ (50 ng/mL) (noted as ++) for 12 hr and finally assessed for cytokine expression and phenotypic response.

Cytokine assessment of pro-inflammatory markers with ELISA demonstrates a significant reduction in inflammation response by cells in a soluble environment with ITA containing materials (FIG. 6B-M). Impact on pro-inflammation signaling compared to PLGA and insert free (Blank) controls with similar trend to solubilized DMI (Soluble) was observed following treatment with ITA+DD (FIG. 6B-G, FIG. 7D), ITA+OD (FIG. 6H-M) and ITA+HD (FIG. 7E-I). Notably, there was a significant reduction in IL-1 (FIG. 6B, H; FIG. 7E) IL-12p70 (FIG. 6F, L) and IFNβ (FIG. 6G) without noticeable reduction in TNFα expression (FIG. 6E, K; FIG. 7F), similar to the mechanistic specificity of ITA described elsewhere⁵⁷⁻⁵⁹. Observed reduction in IL-6 (FIG. 6C, I; FIG. 7G) was marked for soluble DMI but limited in material pre-treatment, suggesting a dose-dependency to mechanistic regulation as shown elsewhere^{57, 58}. Additional assessment of pro-inflammation markers presented reduction in CCL2 (FIG. 6D, J; FIG. 7H) and IL-10 (FIG. 6M; FIG. 7D, I), further suggesting shift to an anti-inflammatory phenotype with ITA treatment. These outcomes align with the mechanistic behavior of soluble ITA that has been published elsewhere.

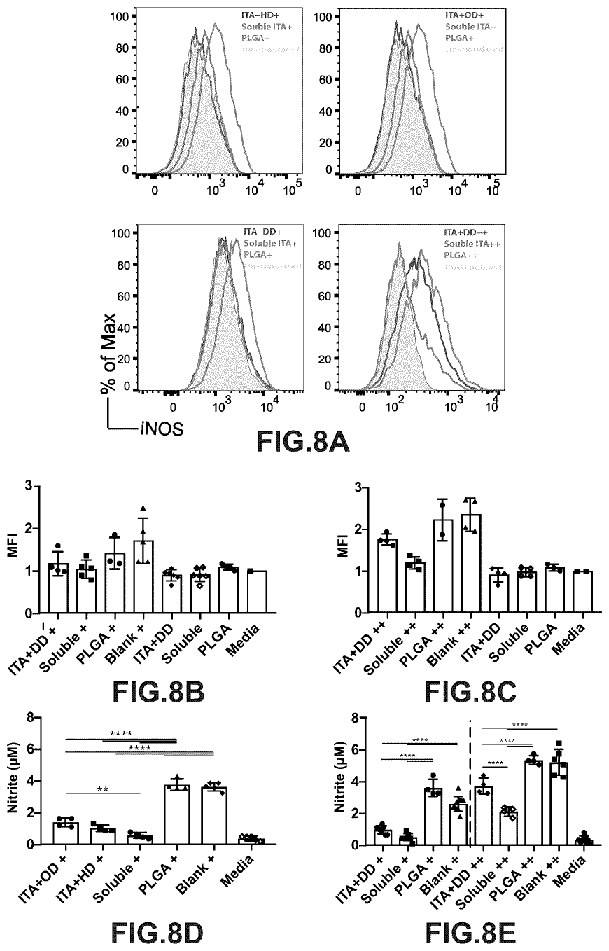

Anti-inflammatory characteristics were prevalent in the phenotypic response of BMDMs with ITA containing polymer pretreatment. Marked reduction in NOS2 expression was observed with LPS stimulation following pretreatment with ITA+HD, ITA+OD and ITA+DD, as well as LPS+IFNγ stimulation following ITA+DD treatment, with similar expression as soluble DMI (FIG. 8A). Mean fluorescence intensity (MFI) of NOS2 expression for ITA+DD pretreated cells presents a quantified reduction in expression when compared to PLGA treated and insert free controls under LPS (FIG. 8B) and LPS+IFNγ (FIG. 8C) conditions. Observed expression corresponds with reduced nitrite production following treatment with gel (ITA+HD, ITA+OD) (FIG. 8D) and solid (ITA+DD) (FIG. 8E) material. Coupled with cytokine assessment, these outcomes suggest the anti-inflammatory behavior with the degradation release of ITA in vitro.

### Reduction of Immune Cell Infiltration in a Peritoneal Injection Model

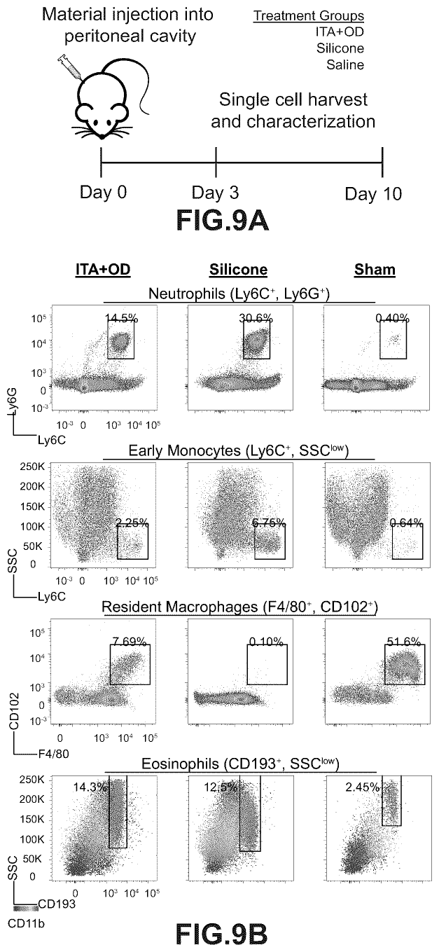

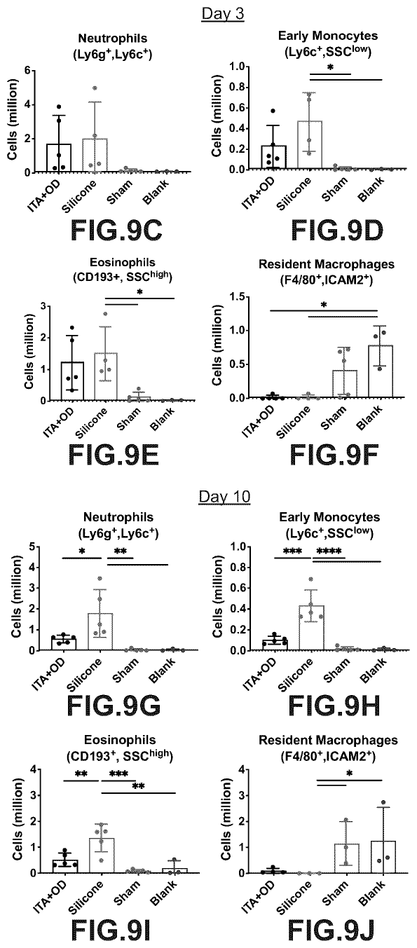

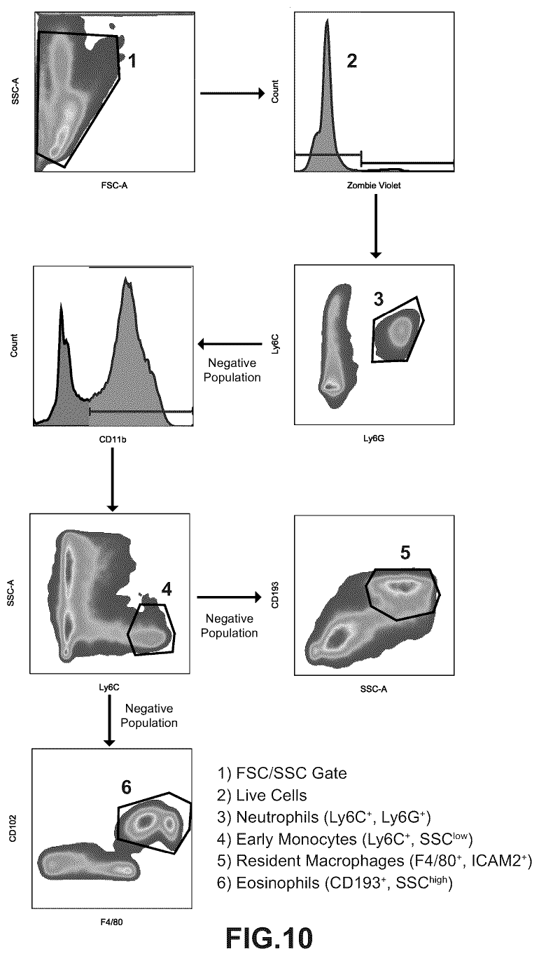

A murine peritoneal injection model was employed to assess immune cell recruitment upon injection of ITA+OD (viscosity: 42.59±0.43 Pa·s), compared to liquid silicone (viscosity:13.99±0.15 Pa·s) and saline controls (FIG. 9A). The peritoneal cavity has a limited resident immune cell population, making it a good location to evaluate infiltration through single cell analysis⁶². Silicone was selected to give material comparison of similar properties (i.e: synthetic, inert, injectable). Due to the known complexity of the host response, we used single cell extraction And assessment with multi-color flow cytometry to identify and quantify populations of neutrophils (Ly6G⁺, Ly6C⁺), early monocytes (Ly6C⁺), eosinophils (CD193⁺), and resident macrophages (CD102⁺, F4/80⁺) (FIG. 10).

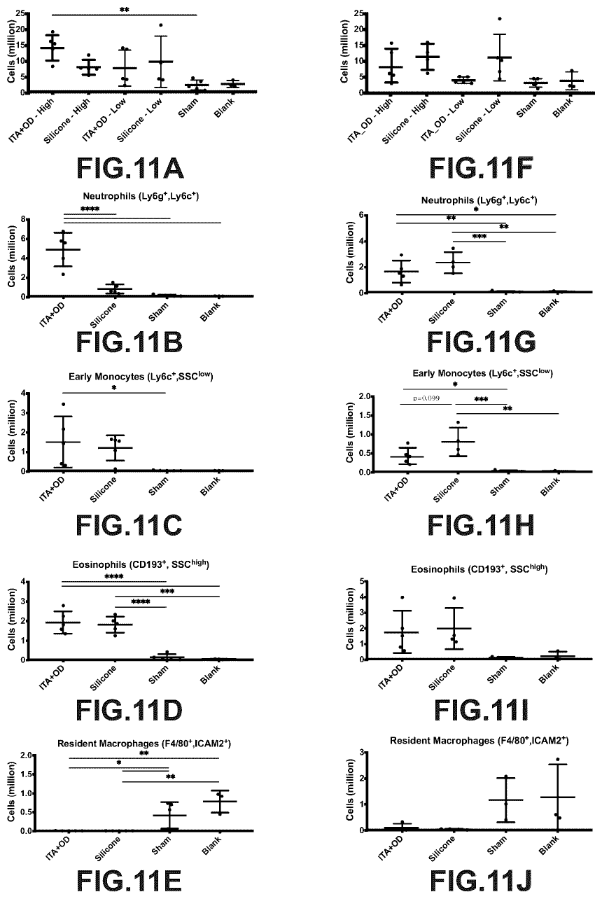

Materials were delivered in high (150 μL) (FIG. 11) and low (50 μL) (FIG. 9) doses, which were selected according to typical injection volumes into the mouse peritoneum. Representative flow cytometry plots of low dose ten days post-injection present decreased immune cell populations in ITA+OD groups in comparison to silicone controls (FIG. 9B). Material implants were quantified for immune cell populations three (FIG. 9C-F, FIG. 11A-E) and ten (FIG. 9G-J, FIG. 11F-J) days post-implantation. Total viable cell number decreased from three (FIG. 11A) to ten (FIG. 11F) days post implantation. Acute response (day 3, low dose) to ITA+OD and silicone was comparable across cell types and material groups (FIG. 9C-F), with demonstrated significant differences from saline (Sham) control.

Cell populations ten days post injection (low dose) present a significant reduction in recruited neutrophil (FIG. 9G), early monocyte (FIG. 9H), and eosinophil (FIG. 9I) populations in ITA+OD groups compared to silicone controls, which remained unchanged from day three assessment. Although limited, ITA+OD recovers a small resident macrophage population which was not present in the silicone groups (FIG. 9B). The impact on implantation of ITA+OD appears dose dependent, with significantly more acute neutrophil recruitment (FIG. 11B) and a reduced trend of infiltrate response compared to silicone ten days post injection.

### Discussion

Typically, synthetic biomaterials have been designed to provide robust mechanical properties while minimizing interaction with the local cell microenvironment⁶³. However, there is opportunity in material design to harness biomimetic strategies to develop polymers that interact with the cell microenvironment, fulfilling some of the roles of immune cells. Here, we have demonstrated a platform technology that relies on the degradation capacity of polyester linkages to provide the stable release of ITA using a novel approach of backbone integration.

The bulk mass stability of ITA containing materials over one month here was notable considering the observed ITA-specific efficacy through molecular degradation. Widely studied polyester materials, such as PLLA, PLGA^{64, 65}, and poly(glycerol sebacate)⁶⁶, exhibit similar bulk degradation properties. Optimization of ITA release rate through modification of synthesis techniques could amplify observed cellular activity. In applications, ITA containing materials appear to accomplish a duality in efficacy, supporting device integration with host immunity while simultaneously supporting infection prevention and allowing for interaction with other cell types of the microenvironment. The synthesis method used here is scalable across other material compositions, inviting the opportunity for co-polymerization with other ester-compatible molecules to broaden the range of mechanical properties.

Assessment of inflammatory regulation of stimulated BMDMs treated with the molecular release of ITA containing materials presented a holistic downregulation of key pro-inflammatory markers. Importantly, the trends observed were comparable to the suggested mechanistic activity of ITA published elsewhere⁵⁷⁻⁵⁹, and followed similar trends to the soluble controls in this study. Comparison of inflammatory regulation ITA polyesters to degradable PLGA, which degrades at similar rates and yields similar acid byproducts, delineated the potential that bioactive efficacy could be derived from material properties (i.e. localized acidity, protein adsorption) that have been reported elsewhere⁶⁷.

Previous work has suggested that key cytokine marker downregulation for ITA include IL-6, IL-1β, and IL-12p70, with a maintenance of TNFα⁵⁷. A concentration dependency to efficacy was shown with DMI treatment⁵⁷, which may explain why ITA materials differentially regulate some cytokine markers from the soluble controls. Importantly, all materials present evident functional down regulation in nitrile production and NOS expression, hallmarks of inflammatory behavior. Intriguingly, we were able to replicate the feedback loop identified between ITA, IFNβ, and IRG1 with ITA+DD pre-treatment⁵⁸.

In the context of biomaterial integration with in vivo host environment, we see an observable downregulation in the key players in inflammatory infiltration in acute biomaterial inflammation⁶⁸. Given that material properties and surface properties have been demonstrated to have an impact on immune response elsewhere⁶⁹, we assessed a comparable silicone material on this basis. There is the potential that differences could be further delineated when considering different material characteristics and microenvironments.

When considering the antibacterial efficacy of ITA material, we saw a stark efficacy of infection prevention with E coli loading in vivo. Surprisingly, there was a similar efficacy observed in the silicone controls. When considered in parallel with the experimental outcomes of the single cell immune cell infiltrate, there may be a level of interplay between the biomaterial host response and infection fighting mechanisms. The increased immune presence observed in the peritoneum three days post implantation, in conjunction with the specific antibacterial efficacy of ITA containing materials may explain the impact on preventing infection, regardless of the inability to prevent E. coli growth on rich media in vitro. This should be considered further in future works with strains of higher clinical risk, wherein the antibacterial characteristics of ITA materials may differentiate them from material controls. In complex in vivo environments, bacterial often grow under substrate limited conditions, further providing basis to ITA effectiveness despite the lack of inhibition on investigated rich substrates.

Further, the role of ITA in pathogenic prevention may not be completely understood, given that multiple strains have identified degradation pathways for the molecule⁷⁰. Importantly, we do not see ITA releasing materials as a replacement to antibiotic therapy, but rather as a method to promote the minimization of infection post implantation. In fact, it has been suggested that the role of ITA in innate immunity may be able to work synergistically with immune cells to restrict persistent growth on substrates such as acetate¹⁵. Importantly, ICL has been shown to be valuable in persistent virulence, which may indicate a greater value of ITA based materials in biomaterial application⁷¹⁻⁷⁴.

Mechanistically, degradation products from ITA based materials lend themselves to a range of proposed immune regulatory mechanisms. As an SDH enzyme inhibitor, one could expect membrane permeability of the small molecule ITA and potential short chain oligomers, as is observed for endogenous itaconate mimics, and through the active transport carrier, dicarboyxlate⁵⁸. Work by El Azzouny et al. suggests the potential for an itaconate extracellular membrane receptor, further lending to the application of itaconate at the material surface⁷⁵. Here, we quantified the release of ITA, but expect that oligomers are also released into the local solution and contribute to polymer efficacy. These may present differential permeability and electrophilic properties, an important aspect in the proposed regulation of the IκBζ-AFT3 axis⁵⁹. Exploration of the role of ITA in activated macrophages, and other immune cells, is still in its infancy⁹. It is possible we are just scratching surface of ITA efficacy, promising potential future opportunity for regulatory ITA based biomaterial therapeutic depots.

Long-term release characteristics of ITA, and the corresponding impact on material properties, should be investigated in future studies. The in vivo results we have demonstrated here ten days post implantation are promising, as we show significant reduction in infiltrated immune cells when compared to a relatively bio-inert control. Coupled with outcomes of E coli survival model, this presents value in application, preventing infection at the surgical site in the short term with support for material integration that minimizes the typical formation of a fibrotic capsule and frustrated response. Furthermore, the mechanistic value of ITA provides a baseline to assessment in specific therapeutic needs beyond general biomaterial adoption that necessitate anti-inflammatory and infection prevention, such as rheumatoid arthritis which involves excessive innate immunity activation⁷⁶. The oral delivery of ITA to rats presented rapid removal within 24 hours, suggesting the need for a sustained delivery strategy⁷⁷. This opens opportunity for biomaterial-based delivery in future studies.

In summary, a family of biomimetic polyester materials based on itaconate was developed to achieve bioactive constructs with antibacterial, anti-inflammatory and nontoxic behavior. Uniquely, this approach allows for sustained delivery of a multifunctional bioactive molecule for modulation of the local cell microenvironment. As biomaterial applications in medicine continue to grow, the basis of a biomimetic technology will facilitate standard and effective innovation with implantation.

### Methods

### Materials

Dimethyl itaconate (DMI), 1,6-hexanediol (HD), 1,8-octanediol (OD), 1,10-decanediol (DD), tin (II) ethylhexanoate, 4-methoxyphenol (MEHQ), chloroform-d (CDCl₃), poly(L-lactic acid) (PLLA), poly(lactic-co-glycolic acid) (PLGA), diethyl succinate, diethyl adipate, LB (Lennox) broth, silver nanoparticles, Lipase from Thermomyces lanuginosus, and lipopolysaccharine (LPS) were purchased from Sigma Aldrich (St. Louis, MO). Methanol (MeOH) and sodium hydroxide (NaOH) were purchased from BioShop Canada (Burlington, ON).

Dulbecco's modified Eagle's medium, RPMI 1640 with L-glutamine, fetal bovine serum (FBS), penicillin-streptomycin, N-2-hydroxyethylpiperazine-N-2-ethane sulfonic acid (HEPES), GlutaMax supplement, Dulbecco's phosphate buffered saline (DPBS), (5-(and-6)-Carboxyfluorescein Diacetate, Succinimidyl Ester) (CFDA-SE), propidium iodide (PI), 4′,6-diamidino-2-phenylindole (DAPI) UltraComp eBeads, ArC Amine Reactive Compensation Bead Kit, ACK lysis buffer and formaldehyde were purchased from ThermoFisher Scientific (Waltham, MA). Mouse recombinant macrophage colony stimulating factor (MCSF) and mouse recombinant interferon gamma (IFNγ) were purchased from Miltenyi Biotec (Bergisch Gladbach, Germany). Greiss reagent kit was purchased from Cell Signalling Technology (Danvers, MA). Stains used in flow cytometry were purchased from Biolegend (San Diego, CA) unless otherwise noted. All materials were used as received unless otherwise described.

### Polymer Synthesis

Polyester materials containing itaconate were prepared using DMI in combination with long chain di-alcohols (i.e. 1,6-hexanediol, 1,8-octanediol, 1,10-decanediol). Monomers were added to a 250 mL triple-neck flask in equimolar amounts with MEHQ (0.5 wt %) as an inhibitor of radical polymerization and tin (II) ethylhexanoate (2 mol %) as a catalyst. The mixture was heated to 130° C. to generate a bulk melt polymerization solution. The reaction solution was stirred at 200 rpm for 6 hours under nitrogen purge followed by a slow reduction of pressure to vacuum and a further 12 hours of reaction, or for times as indicated. Polymers were purified through precipitation in cold methanol (−80° C.) followed by solution decanting and drying of the polymer material. Materials containing other dicarboxylic acids (dimethyl succinate, diethyl adipate) were prepared as described with substitution of DMI with the appropriate carboxylate monomer.

### Polymer Characterization

Polymer structure was confirmed using ¹H NMR on an Agilent DD2 600 MHz spectrometer. Polymer samples were dissolved in CDCl₃. Chemical shifts were tested against the resonance of protons in internal tetramethylsilane (TMS). Peak assessment and integration was conducted using MNova software to validate material structure and determine limitation of undesired radical polymerization. Samples were also characterized by ATR-FTIR (Perkin Elmer Spectrum One). Assessment included 32 scans from 4000 to 550 cm⁻¹ at a resolution of 4 cm⁻¹ and corrections for ATR, baseline and smoothing. Viscosity measurements were performed using a TA Instruments Discovery HR-2 hybrid rheometer. A flow sweep was performed at sheer rates from 0.1 1/1 1/s (7 points, 20° C.)

### Bulk Degradation

To assess mass loss, materials (100 mg) were cast on the base of glass vials (20 mL) of known mass and incubated with 2.5 mL of deionized distilled water for indicated time points. At endpoint, supernatant was collected for mass spectroscopy assessment, then dried (48 hr, 75° C.) and weighted for final mass. Accelerated degradation was assessed under basic (1M NaOH) and solution containing lipase (Thermomyces lanuginosus, 5000 U g⁻¹ polymer) for indicated times using the same method. The degree of degradation was calculated by comparing the dry mass of the polymer film after degradation and the initial mass.

### Mass Spectroscopy Assessment

Liquid chromatography-mass spectrometry (LC-MS) analysis was performed using a Dionex Ultimate 3000 UHPLC system and a Q-Exactive mass spectrometer equipped with a HESI source (all from Thermo Scientific) and controlled by Thermo XCalibur 4.1 software. LC separation was conducted on a Hypersil Gold C18 column (50 mm×2.1 mm, 1.9μ particle size, Thermo Scientific) equipped with a guard column. Solvent A was 5 mM ammonium acetate in water (pH6), and solvent B was 5 mM ammonium acetate in methanol (pH6). The column was run at a flow rate of 300 A μL/min and at a column temperature of 40° C. Autosampler temperature was maintained at 8° C., and injection volume was 10 μL. The gradient was 0-1 min: 2% B; 1-7 min: linear gradient to 98% B; 7-10 min: 98% B; 10-10.5 min: linear gradient to 2% B; 10.5-15 min: 2% B. Data collection was done in positive and negative ionization modes with a scan range m/z 100-1000, resolution 140 000 at 1 Hz, AGC target of 3e6 and a maximum injection time of 200 ms. Standard solutions of ITA ([M−H]−=129.0193), 1,6-HDO ([M+H]+=119.1067), 1,8-ODO ([M+H]+=147.1380), and DDO ([M+H]+=175.1693) were used for validation of retention time and m/z. Molecular release was interpreted as area under respective m/z peaks.

### In Vitro E. Coli Bacterial Growth Assessment

Escherichia coli (BL21) was expanded overnight in LB broth, then seeded (1:100) in fresh medium according to growth conditions. Experiments were carried out in modified M9 minimal medium supplemented with appropriate carbon source (Acetate: 1%, Glucose 0.4%) or LB broth (Lennox). Time course assessment of soluble small molecule inhibition was conducted in 96-well culture plates fitted with a breathable film to encourage aeration and cultured for indicated time with incremental optical density measurement (600 nm). Growth media containing solubilized acid was neutralized with sodium hydroxide (pH=7.0).

To determine polymer antimicrobial properties in solution, six well plates were cast with equal mass polymer samples and polymer free culture plate (polystyrene) controls. Plates were sealed with a plastic film to prevent media evaporation (3 mL/well) and cultured for four days (37° C., 100 rpm) in media conditions as described with daily growth assessment by extraction of (100 μL) to measure change in optical density (600 nm). Silver nanoparticles (10 nm) and PLLA (Mn=85-110 kDa, Sigma) were used as positive and negative controls respectively. Soluble assessment of DMI was conducted using the method described for polymer samples using a 12 well plate format (1.5 mL/well).

### Bone Marrow-Derived Macrophage Culture

Macrophage cells were isolated from 6-12 week old C57BL/6 mice as described previously⁷⁸ and cultured in RPMI-1640 medium with L-glutamine supplemented with 10% heat inactivated fetal bovine serum, penicillin-streptomycin (100 U mL⁻¹) and MCSF (20 ng mL⁻¹) for seven days. For experiments, cells were seeded at 90 000 cells cm⁻² in 6 well culture plates. Cells were treated with ITA+DD, ITA+OD, ITA+HD, or PLGA (coated on glass coverslips; diameter=18 mm) in transwell inserts (8-μm pore size), soluble DMI (0.125 μM), or media only for 12 hr, then activated with LPS (E.Coli O111:B4; 100 ng/mL), or LPS (100 ng mL⁻¹) and IFNγ (50 ng mL⁻¹) for 12 hr prior to assessment. Unstimulated cells treated with identical groups were used as a negative control.

### Quantification Of Cytokines And Nitric Oxide

Cytokine quantification was conducted on cell supernatants using a mouse 10-plex pro-inflammatory assay or an interferon beta single plex assay (Eve Technologies, Calgary, AB) and calculated using their standard curve. Nitrite content was determined using a Greiss Reagent Kit.

### Animal Study Ethics

Experiments in this disclosure involving animals were conducted under the Guide to the Care and Use of Experimental Animals from the Canadian Council on Animal Care and Procedures approved by the Animal Care Committees of the University of Toronto and University Health Network (Toronto, Canada).

### Peritoneal Material Implantation Study

An immune cell infiltration model in the peritoneal cavity was used to assess host response to implanted materials. Injected materials were prepared by sterilizing in 70% ethanol and washed in DPBS followed by drying under sterile vacuum conditions (3 days). Adult C57BL/6 mice (6-8 weeks old, Charles River Laboratories, USA) were injected (low dose: 50 μL, high dose: 150 μL) with ITA+OD, silicone, or saline (Sham) using a 18 G needle into the peritoneal cavity. Three or ten days post implantation, animals were euthanized and the peritoneal cavity was washed with DPBS (10 mL) to extract single cell infiltrates. Cells were concentrated, treated with ACK lysis buffer to remove red blood cells (1 mL, 5 min), washed with DPBS, then counted and assessed by flow cytometry.

### Bacterial Survival Model

ITA+OD and silicone material was prepared as described for the peritoneal infiltration model, then adult C57BL/6 mice (6-8 weeks old, Charles River Laboratories, USA) were injected intraperitoneally (18 G needle) with ITA+OD, silicone, or saline (Sham). Three days post implantation, animals were administered an E. coli (BL21 strain) bacterial load (200 μL, 1×10⁹ CFU per mouse) or saline (200 μL). Animals were monitored every two hours for 12 hours, then at 16, 24, and 48 hours for clinical signs of welfare. Clinical signs included respiration, physical appearance, behavior and body appearance, and a score greater then 21 (or greater then 3 in a respiratory category) indicated the humane endpoint as described elsewhere⁷⁹.

### Flow Cytometry

Cells analyzed by flow cytometry were concentrated (2 million/100 uL), washed with DPBS and stained with Zombie Violet Fixable Viability Kit (1:500; RT, 20 min), blocked with anti-CD16/32 (RT, 10 min), and surface stained according to experimental protocol (4° C., 30 min). In vitro studies with BMDMs were stained with anti-F4/80 and CD11 b, fixed in 4% formaldehyde, permeated (Permeabilization Buffer, eBioscience), and stained for anti-NOS2 (Santa Cruz Biotechnology). Cells were gated for viability and F480⁺/CD11 b+ cells prior to assessment of mean fluorescence intensity (MFI) for NOS2 expression. Peritoneal single cell extracts were surface stained with a multi-colour immune cell panel (Gating: FIG. 10).

Compensation and positive staining were performed for each experimental run with UltraComp eBeads and ArC Amine Reactive Compensation Bead Kit. Flow cytometric assessment was conducted on a BD LSRII-OICR Cytometer (Flow and Mass Cytometry Facility, University Health Network, Toronto, ON). Data acquisition was done with BD FACS Diva software, analyzed with FlowJo (TreeStar) software. Immune cell number in peritoneal extracts was determine as relative abundance against a manual cell count conducted prior to staining.

### Screening Fibroblast Cell Attachment And Viability To Polymer Sheet

To assess cell attachment and viability, ITA+OD was cast in 24-well plates (0.2 mL) to form a thin polymer sheet. Prior to cell seeding, wells were thoroughly rinsed with PBS and sterilized with 70% ethanol (30 min) followed by three washes in PBS. Dermal fibroblasts were seeded in coated wells or uncoated controls (tissue culture polystyrene) at 1.8×10⁴ cells/cm², cultured in DMEM supplemented with 10% fetal bovine serum, Glutamax (1%) and Pen/Strep (100 U mL⁻¹). Two days post-seeding, cell viability was assessed using CFDA-SE (1:1000) and PI (1:200) in DPBS (30 min, 37° C.) followed by fixation in 4% paraformaldehyde (30 min). Cells were counter-stained with DAPI (1:1000) and imaged with an Olympus fluorescent microscope. Adhered viable cells were counted using the IMARIS 8 image analysis and compared between experimental groups.

### Material Coating

To demonstrate material application, ITA+OD was combined with Sudan IV in ethyl acetate (0.8 mg/mL) and coated on the inner wall of silicone tubing with slow rotation during solvent evaporation. Coating of ITA+DD on metal alloy was done by dropwise solvent casting (0.8 mg/mL) dissolved in ethyl acetate.

### Statistical Analysis

All data are presented as the average ±standard deviation (s.d.) and differences with p<0.05 were considered significant. Sample sizes (n) indicate biological replicates or number of animals. Statistical analysis were conducted using Graphpad Prism 8. Normality and equality of variance were tested before a statistical test. One way ANOVA in conjunction with Tukey's Multiple comparisons test was used to determine the statistical significance in pairwise comparisons.

### Two-Step, One-Pot Polycondensation Reaction Generating Tunable Elastic Materials

Synthetic polyester elastomeric constructs have become increasingly important for a range of healthcare applications, due to tunable soft elastic properties that mimic those of human tissues. A number of these constructs require intricate mechanic design to achieve a tunable material with controllable curing. The present disclosure also relates to the synthesis and characterization of poly(itaconate-co-citrate-co-octanediol) (PICO), which exhibits tunable formation of elastomeric networks through radical crosslinking of itaconate in the polymer backbone of viscous polyester gels. Through variation of reaction times and monomer molar composition, we were able to generate materials with modulation of a wide range of elasticity (36-1476 kPa), indicating the tunability of materials to specific elastomeric constructs. This correlated with measured rapid and controllable gelation times. As a proof-of-principle, we developed scaffold support for cardiac tissue patches, which presented visible tissue organization and viability with appropriate elastomeric support from PICO materials. These formulations present potential application in a range of healthcare applications with requirement for elastomeric support with controllable, rapid gelation under mild conditions.

Popularity in application of synthetic polymer elastomers has stemmed from the ability to provide structural and mechanical stability while also mimicking the local dynamics of host tissue microenvironments⁸⁰. Applications can range across biomedical technologies, serving both as mechanical supports for tissue regeneration with and without tissue engineered technologies, and as structures for in vitro organ-on-a-chip devices to better mimic the extra-cellular matrix (ECM) characteristics of native tissues. Examples of synthetic elastomers used in biomedical research include a diverse range of backbone structures such as polyureathanes⁸¹, and polyesters⁸², among others⁸³.

Use of polyester linkages has attracted attention based on the wide range application of polylactones, including poly(L-lactide) and poly(glycolide), in FDA approved applications⁸⁴. Although these exhibit desirable compatibility, they are limited by their mechanical stiffness⁸⁵. As such, effort has been made to develop soft biomaterial polyester elastomers including aliphatic based (e.g poly (lactide-co-caprolactone)⁸⁶, poly(glycolide-co-caprolactone)^{87, 88}), polyhydroyalkanone based (e.g. poly(hydroxybutyrate)⁸⁹), and polyol based (e.g. poly (glycerol-co-sebacate) (PGS)⁹⁰, poly(octanediol-co-citrate) (POC)⁹¹) material constructs.

While these polyester elastomers provide desired mechanical stiffness for soft material application, they are limited by thermoplastic properties that require prolonged heating and/or reduced pressure to generate a branched elastomeric structure. This motivates the inclusion of secondary crosslinking mechanisms in polyester structures to overcome the need for thermal processing. To achieve this goal, photopolymerization is an effective strategy. This has been accomplished previously⁹², including post-polymerization acrylation of PGS^{93, 94}, citrate based materials ⁹⁵, terminally acrylated star-polyesters^{96, 97} and inclusion of unsaturated carbon bonds in the material backbone⁹⁸⁻¹⁰². In our previous works, we have utilized tri-carboxylic acid and di-alcohol based polyesters which include maleic anhydride in their material backbones with great success in in vitro¹⁰³⁻¹⁰⁵ and in vivo applications ^{106, 107}.

Although successful, materials containing maleic anhydride as an unsaturated group require a relatively high crosslink energy to achieve gelation, motivating alteration of the material backbone to include an unsaturated moiety with greater reactivity to radical polymerization. Itaconic acid (ITA) is an unsaturated di-carboxylic acid used widely as a crosslinking agent given the high reactivity of its pendant alkene group¹⁰⁸. Recently, Winkler et al. generated materials possessing unsaturated acrylate bonds available for further functionalization⁶⁰. This provided us with a framework to incorporate this molecule into polyester resins to achieve a two-step elastomer polymerization process with precise control of crosslinking characteristics.

In this disclosure, there is disclosed the synthesis of a polyester elastomer combining citric acid, 1,8-octanediol, and ITA, designated poly(itaconate-co-citrate-co-octanediol), or PICO, to yield a novel polymer gel with the ability for secondary carbon-carbon polymerization through a radical mechanism. Given it is a viscous gel following initial polycondensation, this material can be formed into intricate shapes then cured rapidly to generate a functional elastomeric structure. Herein, the inventors have characterized this material under a number of reaction conditions to demonstrate its tunability of mechanical properties to application. As a functional example of biomaterial application, we demonstrate the successful formation of PICO scaffolded cardiac tissue engineered patches, which require mechanical support for repetitive beating cycles. Developed through a straightforward synthesis procedure, this family of ITA based polyester elastomers presents a wide range of material tunability, both in the context of gelation time and elasticity, yielding controllable gelation under mild curing conditions.

### Results

### Material Development

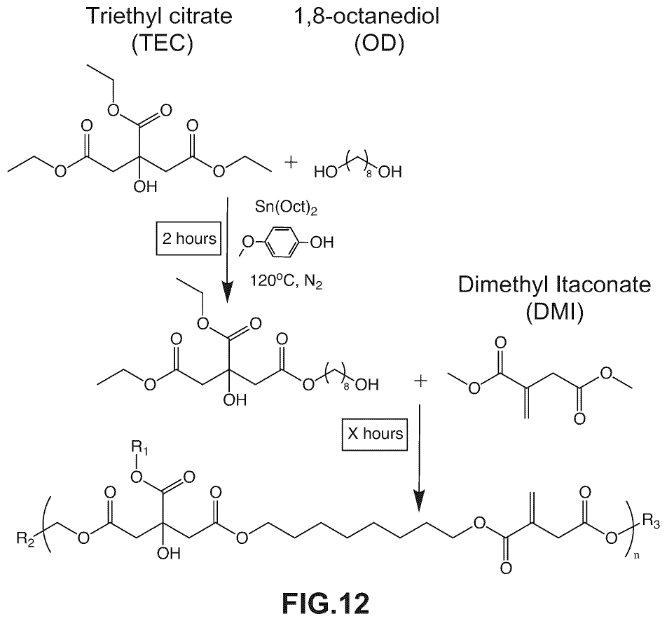

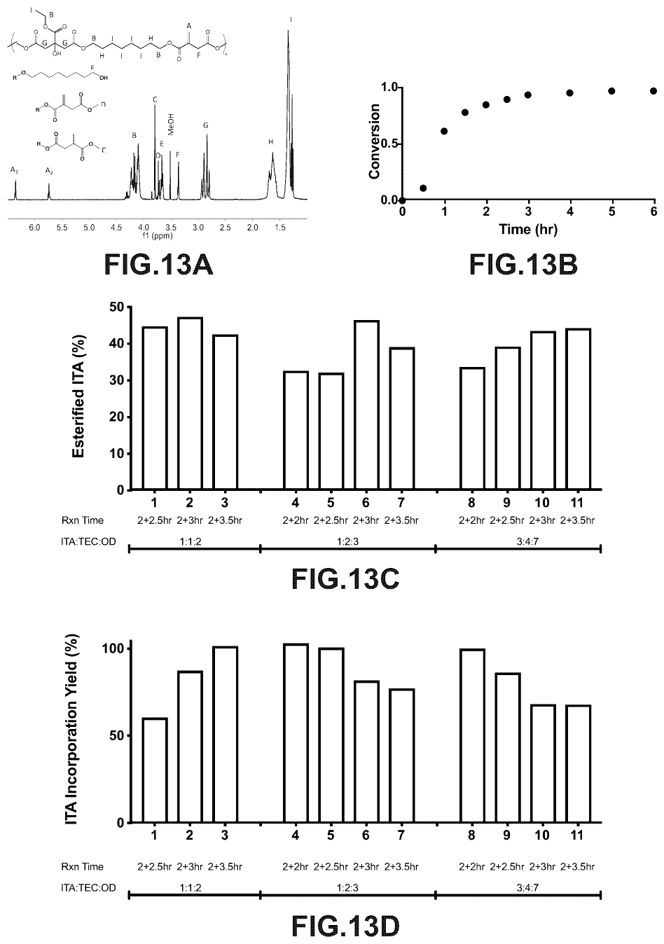

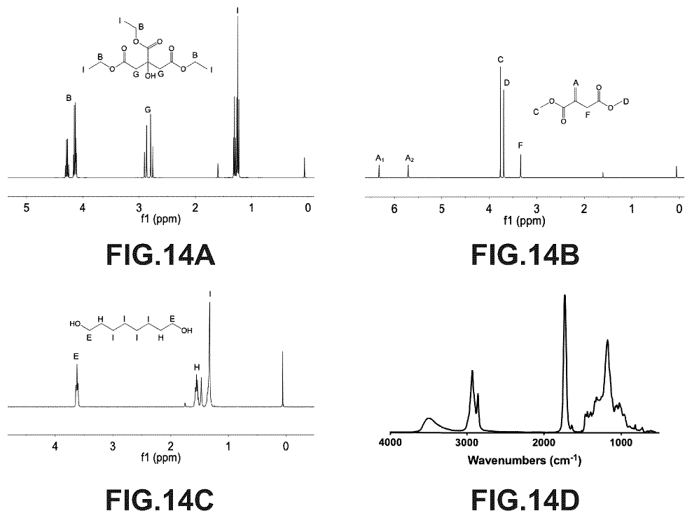

Using a two-step, one-pot polycondensation reaction, carboxylate and alcohol monomers were combined to generate molecular branched viscous polyester PICO gels with release of MeOH and ethanol (EtOH) by-products removed through boiling and nitrogen gas purge (FIG. 12). A two-step process was used to facilitate chain branching with tri-carboxylate TEC and OD, priming chains for reactivity with ITA molecules (DMI). This method maintained the unsaturated groups through condensation (avg: 103±1%), as verified by proton NMR (¹HNMR) (Table 1). A representative ¹HNMR (FIG. 13A) and FTIR (FIG. 14D) present the integration of relevant monomer functional groups into the final purified polymer materials. A time course of OD esterification in the reaction of TEC with OD (1:1 molar ratio), quantified using ¹HNMR, presents a plateau of reactivity at 2 hours, rationalizing the selection of this timing for pre-polymerization prior to DMI addition to polymer combinations (FIG. 13B). Given this finding, we generated a library of materials with variation in monomer feed ratio and reaction time post DMI addition (Table 2). Throughout, the molar feed ratio of OD to carboxylate content (moles of DMI+TEC) was held equal, with variation of citrate to ITA. Materials were generated with an acid feed ratio (DMI:TEC, mol:mol) of 1:1 (Polymer 1-3), 1:2 (Polymer 4-7) and 3:4 (Polymer 8-11). Reaction time post DMI addition was varied from 2 to 3.5 hours.

¹HNMR assessment and quantification was conducted for each material combination (Table 3). We analyzed the polymer content, amount of each carboxylate group (citrate and itaconate) in the polymer relative to OD; monomer incorporation yield, ratio of carboxylate polymer content (citrate or itaconate) to the monomer molar feed ratio (TEC or DMI) on the basis of OD; and the degree of polymer groups that were esterified, the disappearance of peaks corresponding to monomeric endgroups. Esterification of monomers was confirmed by the formation of esterified OD (B, 4.28-4.00 ppm) and disappearance of DMI monomeric end groups (C, 3.78-3.75; D, 3.70-3.67 ppm), TEC (I, 1.40-1.20 ppm), and OD (E, 3.66-3.59 ppm). Esterification of OD (66.8-76.9%, avg:73±3%) remained relatively consistent across polymer groups, with no evident trend related to polymerization conditions. Increase in length of reaction time presented a trend in increased TEC (39.3-55.1% avg:46±5%) esterification, but this was not consistent across polymerization groups.

With focus on this study on the incorporation of pendant unsaturated group on ITA for radical based post-polymerization, we highlight the incorporation and esterification of these groups. Degree of esterification was unrelated to reaction time in Polymers 1-3 (42.2-47.0%) and increased with reaction time with Pol 4-7 (31.8-46.1%) and Pol 8-11 (38.9-43.9%) (FIG. 13C). Esterification of ITA (disappearance of DMI end groups) was preferential to the end groups further from the pendant unsaturated group across all material groups (C: 29±5%, D: 50±7%) (Table 1).

Overall, ITA groups exhibited a low degree of esterification, suggesting they were mainly maintained as end groups on polymer chains. Yield of ITA incorporation increased with reaction time in the high DMI feed group (Pol 1-3) from Polymer 1 (59.9%) to Polymer 3 (101.0%) (FIG. 13D). This trend was inversed in groups with low (Pol 4-7, 100.1-76.9%), and medium (Pol 8-11, 85.9-67.6.9%) DMI feeds. The incorporation of TEC was greater than 100% across groups with no evident trend (102.7-142.5%, avg: 121±10%). This result is attributed to the 2 h pre-polymerization period and the excess of stoichiometric equivalence in reactive groups (3 acid groups—TEC, 2 alcohol reactive groups—OD) with the overall molar equality of acid to alcohol groups. Assessment of molecular weight by gel permeation chromatography presented low molecular weight materials (Mn<6000 Da) with a high dispersity (1.81-7.81) (Table 4). There was no strong correlation between polymerization conditions, but these outcomes further indicate the low degree of polymerization conversion of these materials.

### Crosslinking Characteristics

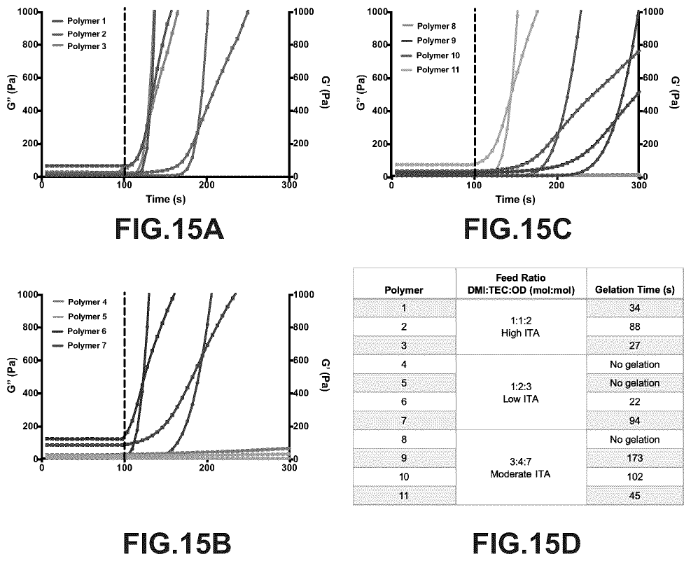

The gelation time of bioelastomer prepolymers was measured using a rotational rheometer outfitted with a UV light apparatus to measure change in storage (G′) and loss (G″) modulus with illumination. Time traces of all samples present stability; G′ and G″ values were stable from the start of data collection measurement and remained constant (t≤100 s) before the crosslinking was induced by UV light (FIG. 15A-C).

Polymer gelation point was considered the point where G′ exceeded G″ as an indication of gel to solid material transition (FIG. 15D). Variation in crosslinking time was seen across groups, with the most rapid and consistent reactivity observed with high ITA content (Polymer 1-3) (FIG. 15A) (Polymer 1: 33.5 s, Polymer 2: 88.2 s, Polymer 3: 27.4 s). Materials with low DMI feed ratio demonstrated variation in reactivity, with no evident crosslinking in low reaction time materials (Polymer 4 and 5) and lack of consistent trend in Polymers 6 and 7 (Polymers 4,5:—no gelation, 6: 21.5 s, 7: 94.2 s) (FIG. 15B). Moderate ITA content materials present a trend of decreasing crosslink time with increased reaction time (Polymer 8: no gelation, Polymer 9: 173.2 s, Polymer 10: 102.2 s, Polymer 11: 45.6 s) (FIG. 15C). Polymers that did not have evident gelation were assessed for 600 s of UV exposure.

### Mechanical Properties

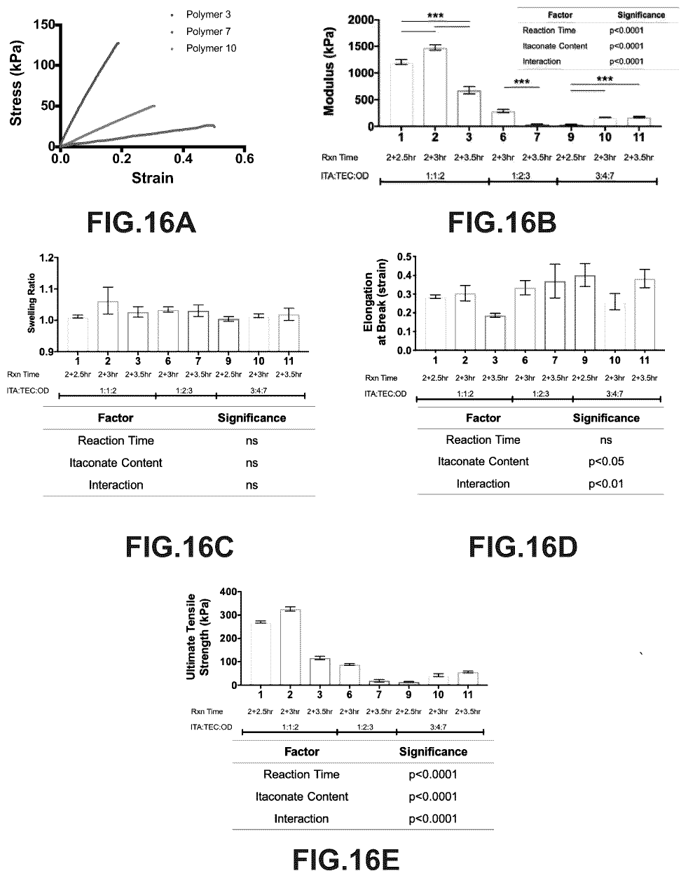

Tensile properties of all polymer groups were assessed using ASTM standard methods. Representative stress-strain curves (FIG. 16A) present modulation of elasticity with change in monomer feed ratios. Crosslinked with a consistent energy (800 mJ), the high DMI feed group (Polymer 1-3) presented a notably higher Young's modulus then the polymers with the low and moderate ITA feed. Young's modulus decreased with reaction time in the high DMI feed group (Polymer 1-3) from Polymer 1 (1204±46 kPa) to Polymer 3 (678±67 kPa) (Polymer 2:1476±52 kPa) (FIG. 16B).

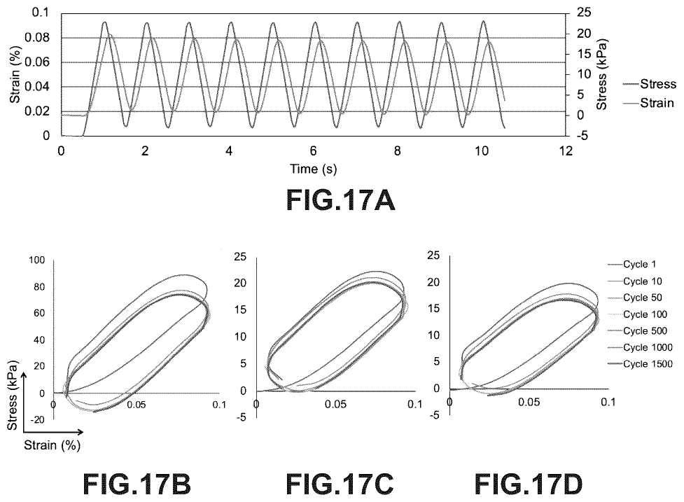

The trend was also observed in the low DMI feed group (Polymer 6: 290±31 kPa, Polymer 7: 41±6 kPa) but reversed in the medium DMI feed group (Polymer 9: 36±9 kPa, Polymer 10: 174±4 kPa Polymer 11: 177±11 kPa). A strong significance (p<0.0001) was calculated for the impact of reaction time and DMI feed content, as well as their interaction effect on elastic modulus. Cyclic loading of polymers 2, 6, and 10 confirmed elastomeric mechanical stability of PICO over time (1500 cycles, 10% strain) (FIG. 17). No significant swelling in water was observed in tested material groups (ratio ˜1.0) (FIG. 16C).

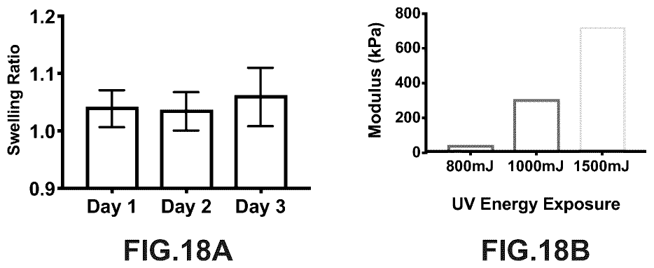

Longer immersion of a representative polymer (Polymer 10) over three days did not change the results of the swelling test indicating that overnight incubation was sufficient to approximate the swelling equilibrium (FIG. 18A). Material elongation at break was not largely impacted with reaction time (non-significant) but showed a slightly significant change with ITA content (p<0.05) and the interaction of this with reaction time (p<0.01), although the magnitude of elongation range between all materials was small (0.19-0.4) (FIG. 16D). Considering the relatively consistent elongation at break, ultimate tensile strength follows a trend directly related to that of modulus and impacted by reaction time and ITA feed (p<0.0001) (FIG. 16E). Elasticity of the polymer was demonstrated to be tunable with changes in crosslink energy, Young's modulus of Polymer 9 increased with crosslink energy from 36 kPa at 800 mJ UV exposure to 298 kPa at 1000 mJ and 717 kPa at 1500 mJ UV exposure (FIG. 18B).

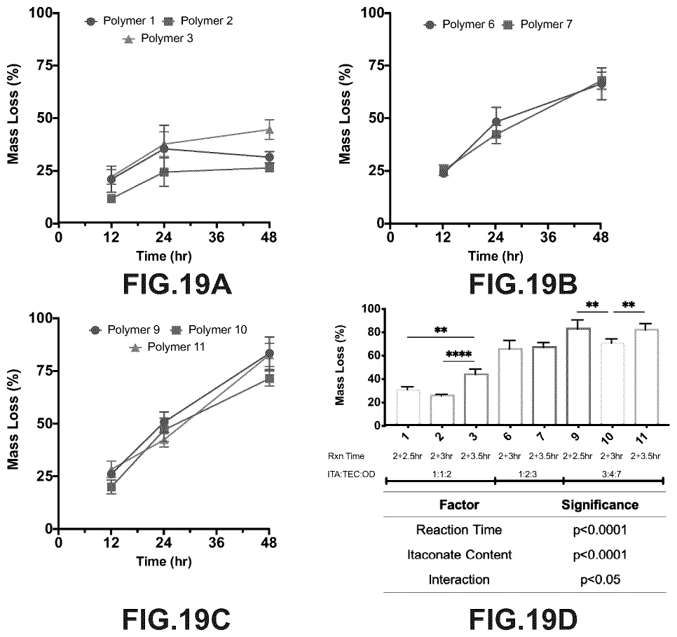

Hydrolytic degradation behaviour of crosslinked PICO materials was assessed under basic conditions (0.25 M, 48 h, 37° C.) to differentiate impact of polymer composition on rate of dissolution. The high DMI feed group materials degraded at a lower rate over 48 h (Polymer 1: 31±3%, Polymer 2: 26±1%, Polymer 3: 44±5%) (FIG. 19A), when compared to low (Polymer 6: 66±8%, Polymer 7: 67±4%) (FIG. 19B) and moderate (Polymer 9:83±8% Polymer 10:71±4% Polymer 11: 82±6%) (FIG. 19C) feed groups. This trend was observed at 12- and 24-hour sample points as well, with an evident plateau in mass loss in high ITA content materials. Comparison of mass at 48 h of exposure highlights the significance of itaconate content (p<0.0001) and reaction time (p<0.0001), as well as their interaction effect (p<0.05) on hydrolytic degradability (FIG. 19D).

### Cardiac Patch Generation and Cell Toxicity Assessment

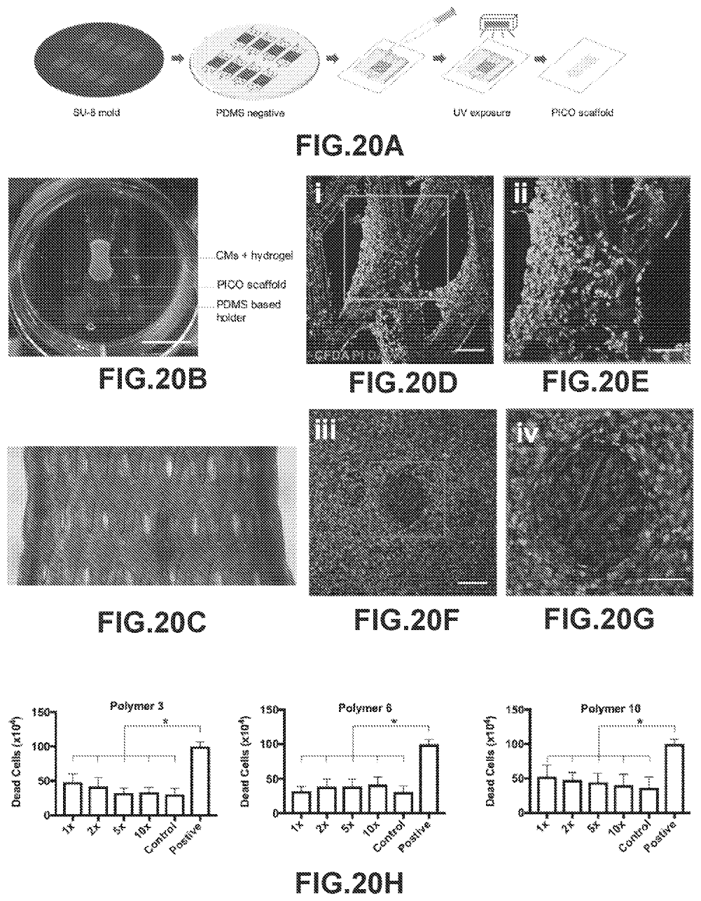

Patches were generated using standard photolithography and microfabrication techniques. PDMS moulds of the desired scaffold shapes were fabricated, into which the polymer was perfused (FIG. 20A). Exposure to UV light was performed and the scaffold shape was retained following removal from the mould. Generation of polymeric scaffolds with roughly 150 μm wide struts containing narrow grooves was successful with this polymer, indicating its applicability in settings where control over features at the micron-scale is required such as in tissue engineering applications. Due to other relevant characteristics of this polymer for cardiac tissue applications, such as its elastomeric properties, evaluation of its use as a scaffold for CMs was undertaken.

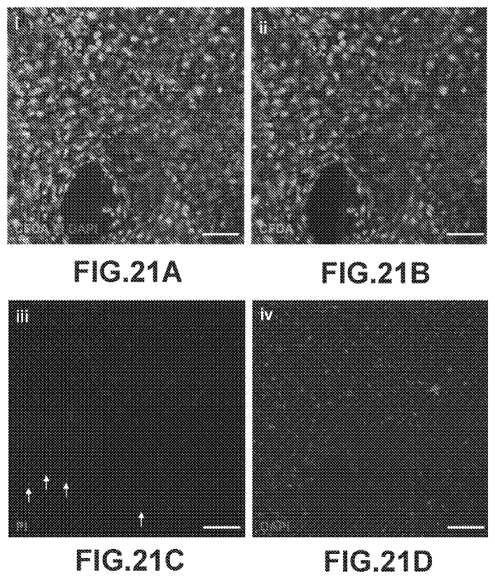

CM-based patches were successfully generated and the crosslinked PICO polymer 10 provided a suitable scaffold for cell attachment and assembly (FIG. 20B). Neonatal rat CMs were able to wrap around the scaffold struts and compacted the hydrogel to form tissue patches (FIG. 20C). CMs on the patches demonstrated spontaneous contraction within two days of cultivation and maintained contraction over four days at which point cell culture was stopped. The polymer scaffold was compressed by the contracting tissue, demonstrating the suitability of this material for a cardiac patch application. Cell viability was observed though CFDA-SE and PI staining which reveals live and dead cells respectively (FIG. 20D). A very high proportion of live cells can be observed, indicating the compatibility of the polymer for use in this fashion (FIG. 21). Regions of high cell density (FIG. 20Diii, iv) and lower cell density (FIG. 20Di, ii) around the scaffold material can be observed.

Assessment of cell compatibility to polymer leachates and degradation products was conducted using a conditioned media method. Polymers 3, 6, and 10 were soaked in complete culture media (15 mg/mL, 2 4 h, 37° C.), that was then diluted (1×, 2×, 5×, 10×) and applied to the cardiac fibroblast monolayers (FIG. 20E). Quantification of cell death by lactate dehydrogenase (LDH) release presented no significant difference in all polymer groups for all dilutions when compared to non-conditioned control. In all groups the dilutions and control presented a significantly lower dead cell count then the positive control (10% Triton-X, 1 h).

### Discussion

Through a one-pot polycondensation reaction, we have synthetized a polyester based elastomer, PICO, with a secondary radical polymerization functionality as a result of unsaturated pendant groups in the material backbone. Crosslinked polyester elastomers, including ring-opening^{86-88, 96, 97, 109} and polycondensation^{93, 110, 111} derived polymer derivatives, have demonstrated extensive efficacy in numerous biomedical applications^{82, 92, 112}. Here, we have leveraged condensation techniques for the addition of ITA to a POC backbone to simplify the implementation of synthesized polyester gels as crosslinked bio-elastomers.

To understand the range of properties achievable with this chemistry, we selected diverse DMI feed ratios that would theoretically result in a high (1:1, DMI:TEC; Polymers 1-3) and low (1:2, DMI:TEC; Polymers 4-7) unsaturated content similar to the range observed elsewhere⁹³. Given this work highlights PICO for the first time, we first developed material feeds with this wide range of DMI monomer feed ratios. With limitations observed in crosslinking characteristics in the low ITA group (Polymers 4-7), we then assessed the moderate DMI feed (3:4 DMI:TEC; Polymers 8-11) as a midpoint between the high and low groups to better understand the potential optimal material properties.

A two-step process facilitated chain branching with tri-carboxylate TEC and OD, priming chains for reactivity with ITA molecules (DMI). Use of methylated and ethylated carboxylic acids provided: a) liquid form monomers; b) ease of leaving group removal (boiling points: MeOH=64.7° C. EtOH=78.4° C.)¹¹³, pushing the equilibrium condensation reaction forward; and c) lower required reaction temperature (T=120° C.). The use of carboxylic acid equivalents would have required higher reaction temperatures to achieve melt polymerization (melting points: ITA=162-164° C. citrate=153° C.)¹¹³, and caused undesired side reactivity of the unsaturated pendant group imparted by ITA (data not shown), consistent with other studies¹¹⁴. With the technique described here, and the inclusion of an MEHQ radical inhibitor, we were able to maintain the unsaturated functionality across all groups.

Without being bound by any theory, the inventors contemplate that the achievement of elasticity is enhanced by the two-step condensation reaction we used here, allowing for generation of chain branching with combination of a di-alcohol with a tricarboxylic structure, a hallmark in elastomeric materials⁹⁹, in advance of forming longer polymer chains. With combination of DMI and TEC with OD at the start of the reaction, we suspect the less desirable ethanol leaving group (compared to methanol) and steric hindrance of TEC would limit its reactivity compared to DMI, preventing the development of an elastomeric polymer bulk. By pre-priming TEC with OD, we achieved greater spacing between stiff radical crosslinking bonds (generated by ITA polymer content), achieving increased chain mobility in final elastomeric structures.

In assessing the optimal properties and tunability of PICO synthesis, the inventors considered both the stoichiometric feed ratios of monomers and the reaction time following the addition of DMI to the reaction mixture. As expected, the inclusion of higher amounts of ITA (Polymers 1-3) lead to a decrease in gelation time, but overall the materials that demonstrated gelation did so in less than 100 s (excluding Polymer 9) under a low powered UV lamp. It was the inventors hypothesis that ITA incorporation into the polymer backbone, would increase with reaction time, in turn decreasing gelation time and they observed an increase in DMI monomer incorporation into the polymer bulk with reaction time in the high ITA content materials, but this trend was reversed with other groups. Rather, a trend of increasing degree of ITA esterification with reaction time was seen in the low (except for Polymer 6) and moderate ITA groups (FIG. 13C). This suggests that stability of ITA in the material backbone is important for crosslinking reactivity and explains the outcomes of this study; materials that exhibited the most rapid gelation (Polymers 3, 6, 11) presented high esterification percentages.

It is possible that this trend is more important with lower ITA content in the polymer material (Polymer 4-11), but with excess itaconate (Polymer 1-3) esterification is a less significant driving force to mechanical crosslinking stability in the context of elasticity. Overall, the esterification degree was low (<50%), suggesting active groups were largely prevalent on the end of polymer chains due to the pre-esterification of OD with TEC. Further, esterification was preferential to the end group further from the unsaturated group, consistent with selectivity observed elsewhere¹¹⁵. Given the high yield values observed in the monomeric incorporation of acid groups in comparison of OD content, coupled with the low degree of esterification, the materials generated here likely present a low degree of polymerization wherein the majority of endgroups are carboxylic acids.

This outcome is attributed to the excess in carboxylate reactive groups in stoichiometric comparison to alcohol groups used here, as has been done in the generation of condensation-based elastomers previously^{90, 116}. Future optimization may suggest consideration of stoichiometric equivalence in reactive groups rather than acid:alcohol content.

Assessment of Young's modulus of crosslinked PICO elastomers highlights the wide-range tunability of this bioelastomer. Materials crosslinked at a consistent energy level present the strong correlation between ITA content and stiffness; high ITA PICO presented a much higher stiffness then those with lower ITA content. This correlates with the characteristics of gelation time, as these materials reached a gelation point faster than lower ITA content materials. With prolonged exposure times, the modulus of materials continues to increase (Polymer 9), inviting the concept of elastomer materials tunable to precise crosslinking characteristics according to application. Besides its advantage for fabrication processes, the polymer's property of rapid crosslinking upon UV exposure becomes even more useful when considering applications such as surgical adhesives or sealants with elastomeric properties, which can achieve function by rapid gelation initiated by UV light in vivo¹¹⁷.

Applications of PICO constructs as tissue specific ECM and scaffolding that match native modulus could include cartilage¹¹⁸ (˜520 kPa), peripheral nerve¹¹⁹ (˜580 kPa), carotid artery¹²⁰ (701-965 kPa) and cardiac¹²¹ (˜372 kPa), among others. As a crosslinked polymer bulk, materials did not exhibit appreciable swelling, which may provide advantage in applications that necessitate structural integrity. Ester linkages in PICO provide advantageous hydrolytic degradability over time, suggesting the potential for material resorption in application. In considering tissue specific application, the demonstrated tunability of degradation rate adds an additional benefit to optimized properties.

As a proof of concept assessment of PICO in generating bio-relevant elastomeric constructs, we generated cardiac patch materials using methods we have described previously¹⁰⁷. Cardiac tissue ECM undergoes cyclic loading in vivo, with a modulus that ranges from ˜10 kPa in diastole to −500 kPa in systole ¹²². In previous work from our group, we demonstrated materials with modulus ˜100 kPa to be advantageous for formation of mature cardiac tissue engineered constructs ⁹⁹, and therefore selected Polymer 10 to form micro-scale patches.

When seeded with neonatal cardiac tissue, we observed robust spontaneous tissue contraction and visible tissue viability (FIG. 20D). The tissue was able to remodel around the structure (FIG. 20B-C), a key feature of healthy cardiac constructs¹²³⁻¹²⁵, and wrap around scaffold struts, suggesting material compatibility (FIG. 20Dii). The non-toxic behaviour was further supported with cardiac fibroblast culture in PICO conditioned media. Cardiac tissue constructs serve as a strong benchmark to relevance of a bioelastomer, as tissues subject the material to repetitive cyclic loading. The maintenance of material properties and appropriateness in cell interaction suggest PICO materials would be beneficial for applications in tissue and organ-on-a-chip engineering.

The described PICO materials should be useful in bio-applications that require highly tunable elasticity for soft material applications. Diverse needs for soft materials motivate the desire for precision and modulation of elastomeric properties, notably in the context of high precision physical and mechanical properties. Given the short gelation times, application in 3D printing of constructs could be relevant, allowing for generation of high throughput organ-on-a-chip devices or highly detailed implantable medical devices. Future work should focus on further optimization of the synthesis strategy, including the length of pre-polymerization without ITA monomer and the application of vacuum pressure to push the overall esterification forward. Further, inclusion of other carboxylic and diol molecules might broaden the range of material properties for application. The prevalence of pendant functionality of both free hydroxyl (citric acid) and unsaturated alkene bonds also opens possibility to functionalization of materials with other active groups, further emphasizing the potential of these polymer constructs^{60, 126, 127} Here, we focus on the understanding of material synthesis and properties of this family of polymers, but future consideration should investigate the in vivo behaviour to fully assess applicability. PICO material functionality provides interesting opportunities in application of synthetic bioelastomers.

### Experimental Section

### Materials

Dimethyl itaconate (DMI), 1,8-octanediol (OD), triethyl citrate (TEC), stannous octanoate, 4-methoxyphenol (MEHQ), chloroform-d (CDCl₃), 2-hydroxy-1-[4(hydroxyethoxy)-phenyl]-2-methyl-1 propanone (Irgacure 2959), medium M199x, PluronicF-127, and sodium bicarbonate were purchased from Sigma Aldrich (St. Louis, MO). Methanol (MeOH) and sodium hydroxide (NaOH) were purchased from BioShop Canada (Burlington, ON). Polydimethylsiloxane (PDMS, Sylgard 184) was purchased from Dow Chemical (Midland, MI). Dulbecco's modified Eagle's medium, Hank's buffered saline solution (HBSS), fetal bovine serum (FBS), penicillin-streptomycin, N-2-hydroxyethylpiperazine-N-2-ethane sulfonic acid (HEPES), GlutaMax supplement, Dulbecco's phosphate buffered saline (DPBS), (5-(and-6)-Carboxyfluorescein Diacetate, Succinimidyl Ester) (CFDA-SE), propidium iodide (PI), and formaldehyde were purchased from ThermoFisher Scientific (Waltham, MA). Neonatal Heart Dissociation Kit was purchased from Miltenyi Biotec (Bergisch Gladbach, Germany). Rat tail collagen type I (8.34 mg mL⁻¹) and GFR-Matrigel were purchased from Corning (Corning, NY). All materials were used as received unless otherwise described.

### Polymer Synthesis

Polymer groups were synthesized using a one-pot condensation reaction to generate a viscous gel material. A mixture of OD (10 g), TEC (as defined in Table 1), stannous octanoate (1% mol/mol ester bond), and MEHQ (0.5% wt to all reactants) were combined in a two necked round bottom flask (125 mL) fitted with a water condenser and collection flask. An overall molar ratio of acid (TEC+DMI) to alcohol (OD) of 1:1 was maintained in all reactions. This mixture was reacted at 120° C. for 2 hr with stirring (200 rpm) and nitrogen flow, followed by the addition of DMI (as defined in Table 1) with an additional reaction of 2 to 3.5 hr. The crude polymer was precipitated in ice cold methanol, decanted and dried for 48 hr before use. As needed, materials were prepared for radical crosslinking initiated by ultraviolet (UV) light by mixing with Irgacure 2959 (1% wt) at 60° C. and crosslinked with exposure to a specific amount of UV energy using a UVP Crosslinker CL-1000 L (Analytik Jena).

### NMR Assessment

Purified polymer gels were dissolved in CDCl₃ (10 mg/mL) and analyzed by proton nuclear magnetic resonance spectroscopy (¹H NMR) with a 500 mHz spectrometer (Aligent, USA) and analyzed using MestReNova software. Specific material molecular properties were determined through peak integration according to the following equations, similar to work published elsewhere¹²⁸, with peak assignment according to the representative spectra seen in FIG. 13A. Peak integration range corresponding to each label is summarized in Table 1, and is further confirmed by the ¹H NMR of each monomer (FIG. 14A-C) First, peak/was delineated into signal corresponding to TEC endgroups (I_{TEC}) and backbone OD (I_{OD}) as outlined in the supplemental methods. Given this, purified polymer percentage of acid content normalized to diol was determined for ITA (eqn. 1) and citrate (eqn. 2), and corresponding monomer incorporation yield calculations by comparison to monomer feed (eqn. 3). Esterification of monomers was determined through calculation of end groups present for citrate (eqn. 4), ITA (eqn. 5) and OD (eqn. 6) as a measure of degree of polymerization.

$$ITA polymer content = \frac{4 F}{{I}_{O D}} \times 100 \% \qquad ( 1 )$$

$$Citrate polymer content = \frac{2 G}{{I}_{O D}} \times 100 \% \qquad ( 2 )$$

$$Monomer incorporation Yield = \frac{polymer content}{monomer feed} \times 100 \% \qquad ( 3 )$$

$$\% {Esterified}_{citrate} = ( 1 - \frac{4}{9} \frac{{I}_{TEC}}{G} ) \times 100 \% \qquad ( 4 )$$

$$\% {Esterified}_{ITA} = ( 1 - \frac{( C + D )}{3 F} ) \times 100 \% \qquad ( 5 )$$

$$\% {{Esterified}_{OD}}_{=} ( 1 - \frac{2 E}{{I}_{O D}} ) \times 100 \% \qquad ( 6 )$$

### Elastomeric Testing

Tensile properties were assessed using dog-bone samples according to ASTM D638-14 standard (width:5 mm, thickness:3 mm). Samples were prepared by first generating a PDMS negative from a 3D printed mould and capping it with a glass slide. Moulds were injected with polymer gels containing Irgacure 2959, then crosslinked with 800 mJ cm⁻² of energy unless otherwise specified. Samples were soaked overnight in DPBS before testing. Tensile testing was conducted to failure under wet conditions using an Electroforce 5200 Biodynamic Test Instrument (BOSE) with strain rate of 0.1 mm s⁻¹ and collection of force displacement data was completed using WinTest software. Bulk modulus was determined using data from the first 10% strain. Ultimate tensile strength and elongation at break were collected from the breaking point.

### Swelling Measurement

Swelling of crosslinked polymer samples was quantified at 37° C. with samples that had been crosslinked as described for elastomeric property assessment. Dry polymer mass was recorded, then material was submerged in DPBS and incubated overnight (or for time described in multi-day assessment). Final mass was determined by carefully removing excess DPBS by blotting before collecting value. The swelling ratio was calculated by eqn. 7.

$$Swelling ratio = {m}_{f} / {m}_{d} \qquad ( 7 )$$

where m_{f} is wet polymer mass and m_{d} is dry polymer mass.

### Polymer Gelation Properties

The gelation time of prepolymer gels was determined using viscoelastic rheology measurement techniques. Polymer gels with Irgacure 2959 were prepared as described and placed on the Peltier plate module (25 mm) of a TA Instruments rotational rheometer (TA Instruments, Discovery HR-2), customized to be fitted with a UV lamp (365 nm, Thorlab) under the plate holder (gap: 1 mm). To analyze gelation, a time sweep was used (Frequency: 1 Hz, Strain: 1%, 25° C.) with collection of storage (G′) and loss (G″) moduli. Samples were allowed to equilibrate in the instrument for 5 min, then data collection was initiated 100 s prior to UV lamp illumination. The moduli were plotted, and gelation time was reported as the time of curve crossover post-UV lamp exposure.

### Polymer Degradation

Degradation of crosslinked polymer samples was quantified at 37° C. under accelerated conditions (0.25M sodium hydroxide) with samples that had been crosslinked as described for elastomeric property assessment. Initial dry polymer mass was recorded, then material was submerged in degradation solution (10 mL) and incubated for 12, 24 or 48 hr (37° C., 100 rpm). At endpoint, materials were removed, washed twice in deionized distilled water, then dried before measuring final mass. Mass loss was calculated by eqn. 8.

$$Mass Loss = ( {m}_{i} - {m}_{f} ) / {m}_{i} \qquad ( 8 )$$

where m_{f} is final polymer mass and mᵢ is initial polymer mass.

### Neonatal Rat Heart Cell Isolation

Heart tissue from neonatal rats was isolated as described previously according to an approved protocol through the University of Toronto Animal Care Committee^{99, 107}. Sprague-Dawley neonatal (1-2 days old) rats were euthanized and hearts collected in HBSS. Following the removal of the vena cava and aorta, the hearts were quartered and rinsed four times in HBSS. Digestion, including red blood cell lysis, was performed using GentleMACS Dissociator (Miltenyi Biotec) and Neonatal Heart Dissociation Kit according to the manufacturer's protocol. Cells were then incubated for 1 hour and cardiomyocyte (CM)-rich supernatant was collected. The culture of rat CMs was conducted in Dulbecco's modified Eagle's medium containing glucose (4.5 g L⁻¹) with 10% (v/v) FBS, 1% (v/v) penicillin-streptomycin (100 mg mL⁻¹), 1% (v/v) HEPES (100 U mL⁻¹), and 1% GlutaMax supplement.

### Cardiac Patch Development

Polymer scaffolds were made according to previous work¹²⁹. Briefly, PDMS moulds of scaffold patches were fabricated from SU-8 master moulds that were generated using standard microfabrication techniques, and subsequently capped onto glass slides. Polymer 10, selected on the basis of matching cardiac ECM elasticity, mixed with Irgacure 2959 as described, was perfused into the moulds and exposed to 800 mJ/cm² UV light. The PDMS cap was removed and scaffolds were soaked in DPBS for 1 hour, sterilized in 70% (v/v) ethanol overnight, and washed and soaked in DPBS for 3 hours. Scaffolds were coated with 0.2 wt % gelatin in PBS at 37° C. for 1 hour to improve cell attachment. A PDMS-based scaffold holder was also fabricated in a six well plate according to previous work ¹⁰⁷, soaked with 5 wt % PluronicF-127 in DPBS to prevent cell adhesion and rinsed once with DPBS. Gelatin-coated scaffolds were placed onto the PDMS-based scaffold holders and pinned in place using 0.15 mm diameter minutien pins (Roboz Surgical) prior to cell seeding.

Pelleted CMs were suspended in a collagen-based gel at a density of 100 million cells per mL. The collagen-based gel was made as previously described¹⁰⁵, from rat tail collagen type I with a final collagen concentration of 3 mg/mL in deionized water with 10% (v/v) M199 (10×), 10% (v/v) sodium bicarbonate (2.2 g L⁻¹), and 15% (v/v) Matrigel. The gel was neutralized with 1 M NaOH. According to previous work¹⁰⁷, 25 μL cell suspension was pipetted onto the scaffold and incubated at 37° C. for 20 minutes to allow for gelation, after which an additional 25 μL cell suspension was pipetted onto the scaffold and incubated for 40 minutes. Pre-warmed cell culture media was then added to the scaffolds and placed in an incubator. Culture medium was changed every two days.

After four days, cells on the scaffolds were labeled with CFDA-SE (1:1000) and PI (1:75) in DPBS at 37° C. for 30 min. Patches were fixed overnight in 4% formaldehyde at 4° C. Before imaging, cells were stained with DAPI (1:500) in DPBS for 30 minutes at room temperature. Confocal images of the cardiac patch tissues were captured with a Nixon A1R+ laser scanning confocal microscope in the University Heath Network Advanced Optical Microscopy Facility.

### Cytotoxicity Assessment

Assessment of the cell toxicity of PICO leachate and their degradation products was conducted using a conditioned media approach. Crosslinked polymer samples were prepared as described for elastomeric property assessment, then soaked in complete rat CM media (15 mg polymer/mL) for 24 hr at 37° C. to generate conditioned media. To assess toxicity, primary rat cardiac fibroblasts were seeded in 24 well plates (10⁴/cm²) and allowed to attach overnight. Cells were then treated with dilutions of conditioned media (1×, 2×, 5×, 10×) in complete rat CM media for 24 hr. Cell death was quantified and compared to an untreated and positive (1% Triton-X, 1 hr) control using a lactate dehydrogenase (LDH) Cytotoxicity Assay Kit (Cayman Chemical Company) per manufacturer's instructions. LDH presence in cell supernatants was correlated to cell number using a calibration curve of LDH release to cell death.

### Statistical Analysis

Normality and equality of variance were tested using Graphpad Prism 8.0. Two-way ANOVA followed by pairwise comparisons with Tukey's multiple comparisons test method were used to determine the statistical significance and assess the interactive effects of factors in FIGS. 3 and 5. One-way ANOVA followed by Dunnett's multiple comparisons test were used to determine the statistical significance and pairwise comparisons of FIG. 6E.

### Results

### Generation of ITA Polymer Microparticles For Intracellular Delivery

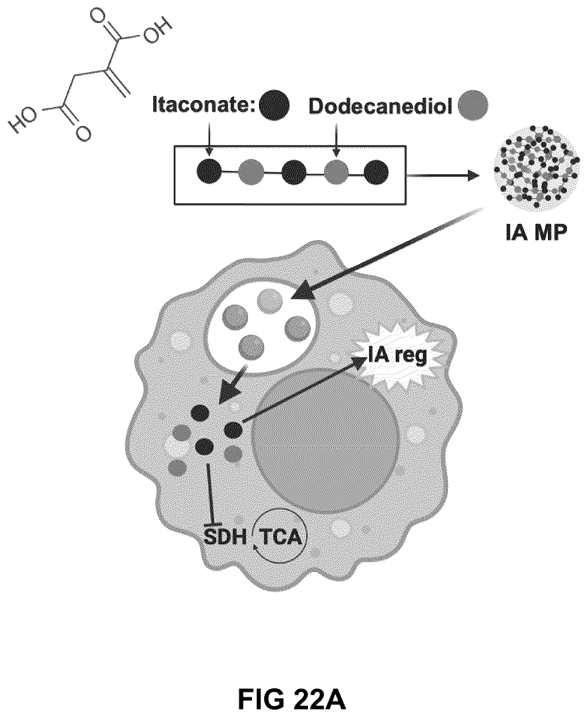

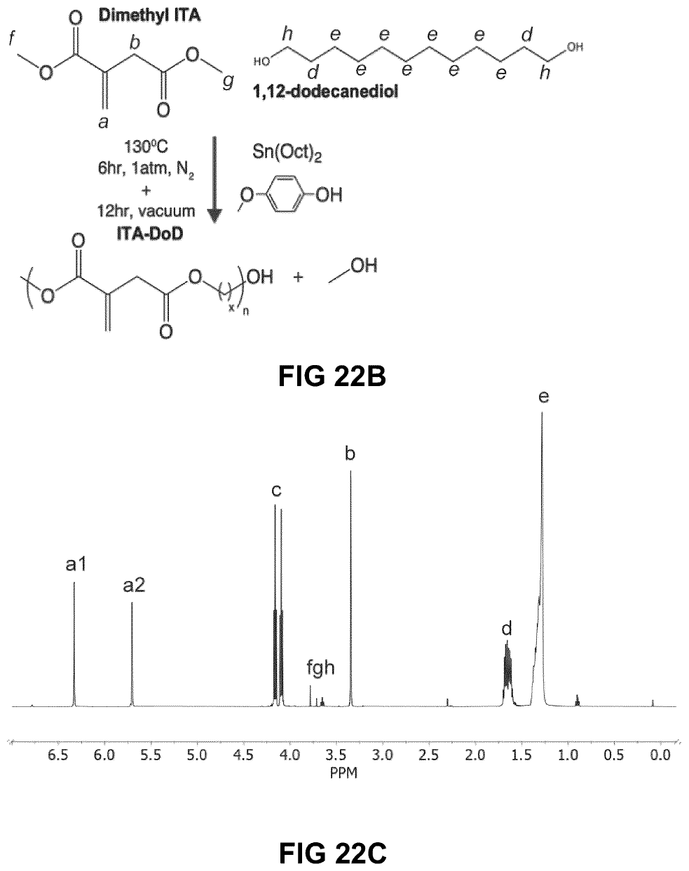

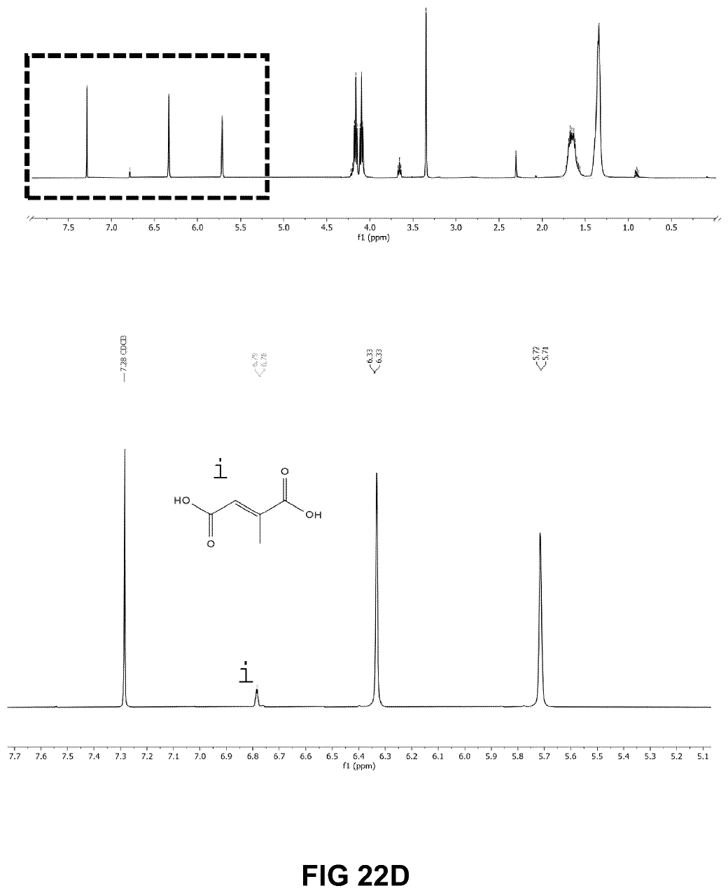

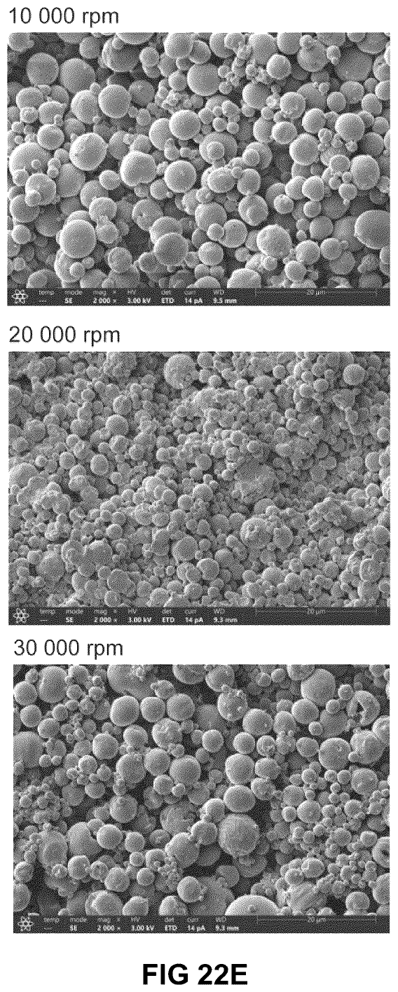

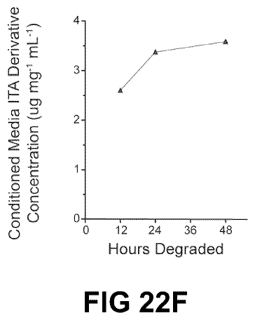

In this disclosure, the present inventors also describe the optimization of ITA-polymer technologies for the development of polymer microparticles (ITA-MP) as a potential therapeutic strategy to target pro-inflammatory macrophage activity with ITA (FIG. 22A).

Phagocytose microparticles were designed for intracellular degradation and metabolite release, based on previously developed polyester ITA-based materials³² and first optimized for molecular weight (MW) and melting temperature (FIG. 22B). DMI was reacted with 1,12-dodecanediol (DoD), in the presence of MEHQ as an inhibitor of radical polymerization and tin (II) ethylhexanoate as a catalyst, in a bulk condensation approach to generate poly(itaconate-co-dodecanediol) (poly(ITA-DoD)). Poly(ITA-DoD) was a solid material at body temperature (37° C.; melting temperature: 50° C.), suggesting its applicability for application in particle development. Structure assessment of poly(ITA-DoD) polymer material using proton nuclear magnetic resonance (H NMR) spectroscopy and subsequent peak identification confirmed the formation of a linear polymer material with maintenance of the pendant unsaturated carbonyl on ITA that drives much of the immunoregulatory capacity (FIG. 22C). Quantification of polymer end groups through peak integration indicated an esterification of ITA and DoD of 96.57% and 100% respectively (Formulas 1-2), suggesting ITA end capped the generated polymer chains. Molecular weight assessment measured number average molecular weight of poly(ITA-DoD) of 10 550±1500 Da and a dispersity of 2.18.

¹H-NMR spectrum data of bulk polymer formed by condensation of dimethyl itaconate and 1,12-dodecanediol also demonstrated spontaneous isomerization of backbone ITA residues to mesaconate (represented by the peak ‘i’), suggesting a degree of spontaneous interconversion between ITA and its isomers during degradation (FIG. 22D). It has been suggested that citraconate and mesaconate (isomers of ITA) have specific immunomodulatory effects^{130, 131}.

The condensation of multiple diols in combination with a dodecane-containing moiety can also provide tunability and thus optimization to physicochemical parameters of the resulting polymer without fundamentally altering its application^{132, 133}. This tunability also extends to the use of a mixture of diols within a single formulation¹³⁴⁻¹³⁶. In some embodiments, this mixture of diols may comprise about ≥25% molar ratio of 1,12-dodecanediol, and more preferably ≥50% molar ratio 1,12-dodecanediol.

Using a water-in-oil-in-water emulsion method, we generated spherical polymer microparticles from poly(ITA-DoD) material (FIG. 22E). To target the biologically active range of macrophage phagocytosis, particles were designed to be within 0.1-10 μm in diameter¹³⁷. ITA release in polymer supernatant treated with deionized distilled water was quantifiable, and increased over a 48 h time period (FIG. 22F). ITA-MP particle size was inversely related to homogenization speed, with average MP diameters of 3.5 μm (10 000 rpm), 2 μm (20 000 rpm) and 1.5 μm (30 000 rpm) (FIG. 22E), suggesting that speed of homogenization in particle production is inversely related to particle size, and potential for future manipulation of particle properties. Micro- and nanoparticulate forms of polyester materials common to biomedical applications can be generated by a variety of methods at sizes of micrometers to the tens of nanometers; these formulations are well-suited to applications such as targeted drug delivery or extended release of active molecules.¹³⁸⁻¹⁴⁰ In some embodiments, the ITA-MP particle size may range from about 25 nm to about 100 μm. Thus, it should be understood that reference to a “microparticle” is not intended to limit the disclosed ITA-based polymeric material to strictly micrometer sizes, and the present disclose encompasses ITA-based polymeric material in nanometer sizes as well.

### ITA-MPs Are Internalized And Non-Cytotoxic

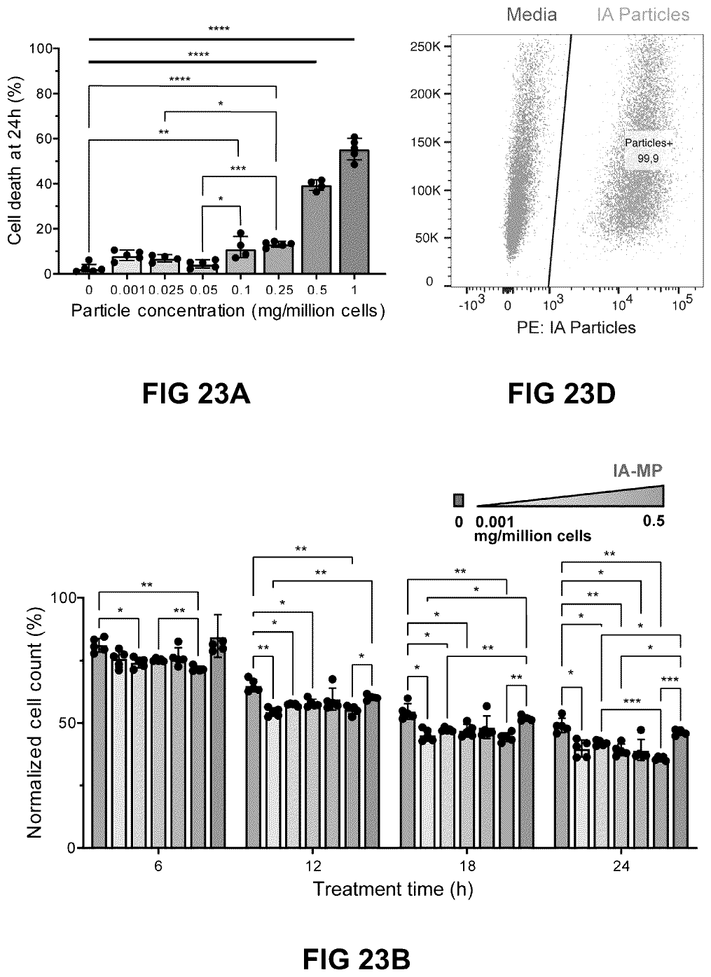

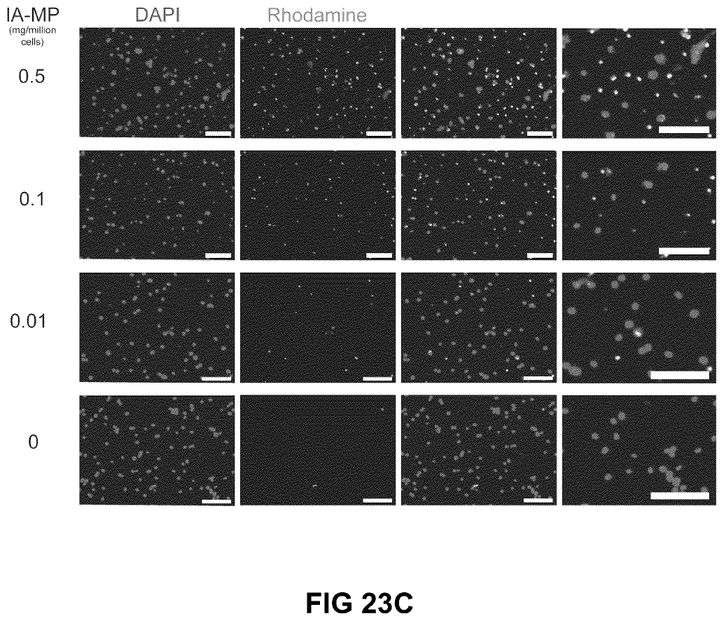

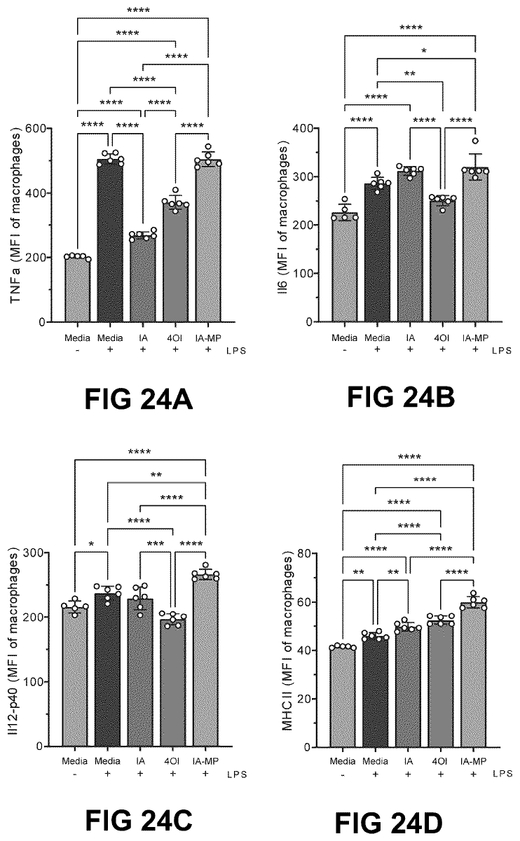

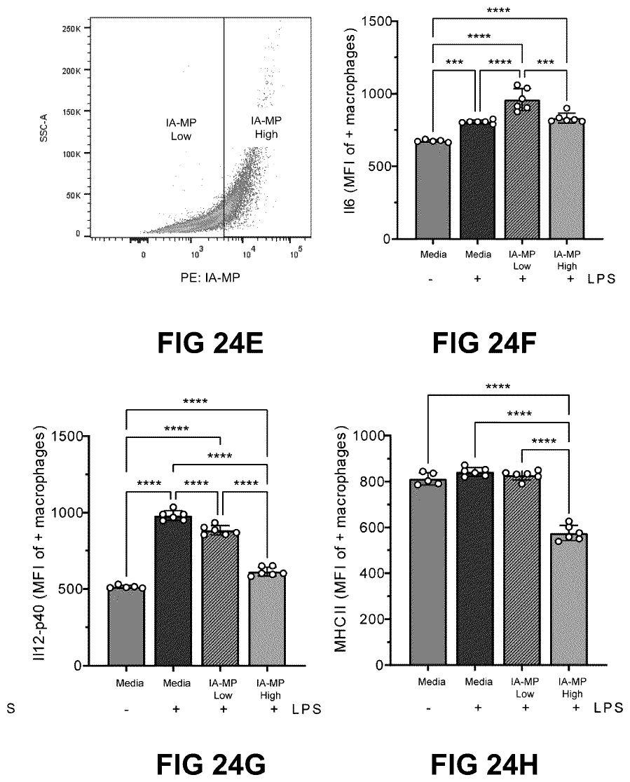

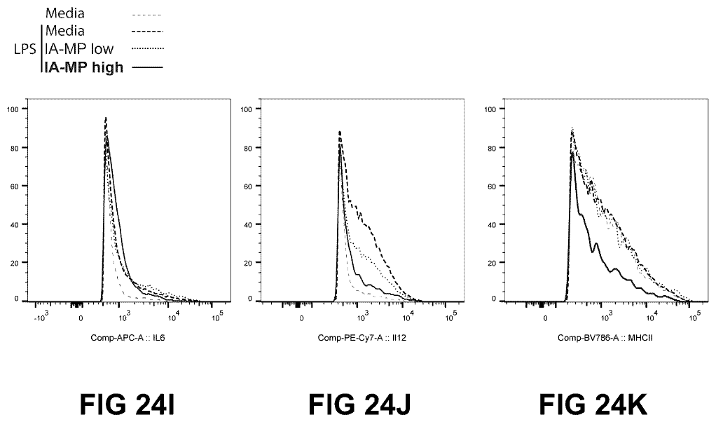

Live cell imaging of macrophages exposed to ITA-based polymeric microparticles at varying concentrations (0.5, 0.25, 0.1, 0.05, 0.025, 0.01, and 0.001 mg·10⁶ cells⁻¹) was conducted to evaluate particle phagocytosis, cell viability, and apoptotic behavior at 24 h of treatment (FIG. 23). Quantitative cell death staining revealed significant increases in cell death in treatments with 0.5 and 1 mg of ITA-MP·10⁶ cells⁻¹ compared to lower particle concentrations and the LPS media control (FIG. 23A), indicating a threshold beyond which particle loading induces cytotoxic effects. Cell counts achieved through DAPI nuclear labelling and serial quantification normalized to initial values showed minor significant decreases in particle-treated groups relative to the LPS media control (FIG. 23B). Representative endpoint images at concentrations of 0, 0.5, 0.1, 0.01 mg·10⁶ cells⁻¹ confirmed this trend of increased cell loss with higher particle concentrations (FIG. 23C). After 24 hours of treatment with 0.1 mg·10⁶ cells⁻¹, 99.9% of BMDMs exhibited successful phagocytosis (FIG. 23D); demonstrating that efficient uptake can be achieved without substantial induction of cell death.

### Macrophage Immunomodulation By ITA-Based Microparticle Degradation

Flow cytometric analysis was undertaken to quantify macrophage ITA-MP uptake and characterize the resultant cell phenotype (FIG. 24). Quantification of inflammatory cytokine expression indicated unchanged TNF-α MFI in ITA-MP-treated BMDMs compared to the LPS media control, which diverged from the results observed with 4OI and soluble ITA which both demonstrated a significant reduction in TNF-α expression (FIG. 24A). ITA-MP treated BMDM demonstrated a significant upregulation of IL-6 expression (FIG. 24B) compared to a stimulated untreated control, consistent with the effect observed from soluble ITA. This trend was similar in the MFI of IL-12p40 (FIG. 24C), and MHC-II (FIG. 24D), which were significantly upregulated in ITA-MP treated cells. Although an average increase in MFI was observed in these markers, up-regulation and distribution of expression was observed was noted to be dependent on particle loading (FIG. 24E-H). To fully appreciate ITA-MP loading specific functionality, we isolated cells positive for expression of IL6, IL12p40, and MHCII, then subsequently gated macrophages based on absolute internalization of ITA particles determined by PE intensity (rhodamine signal from particles) vs side scatter signal (FIG. 24E). This provided a population for analysis of high and low particle loading, appreciating ITA-MP treated macrophages previously determined to have nearly 100% particle uptake at this concentration. Quantification of MFI in the high and low group, contrasted to particle free controls with and without LPS stimulation provided insight into the role of particle loading on relative expression. Expression of IL-6 (FIGS. 24F,I), IL12p40 (FIG. 24G,J), and MHCII (FIG. 24H,K) were significantly reduced in the high ITA-MP group when compared to low ITA-MP loaded cells. Quantified MFI of expression in the high ITA-MP group was notably reduced in IL12p40 and MHCII expression, which were significantly reduced compared to particle free LPS stimulated controls.

### Macrophage Metabolic Dependencies Respond to ITA-MP Administration

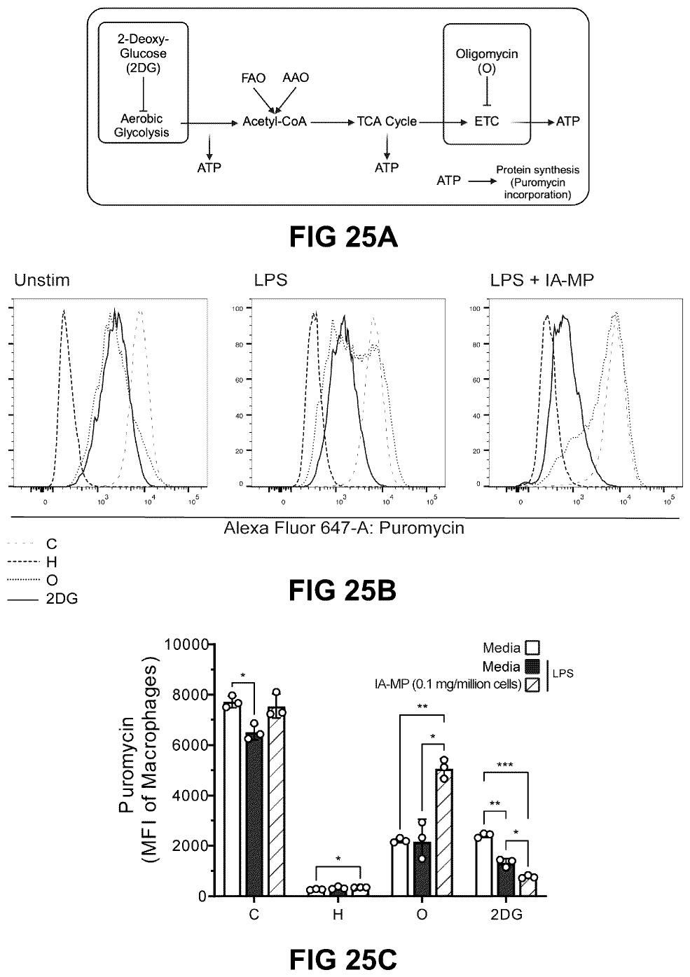

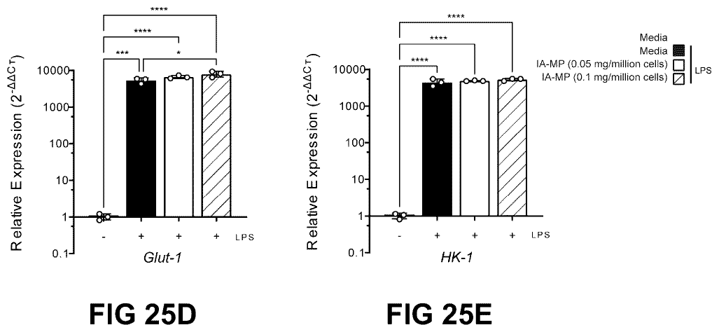

The SCENITH approach to characterization of metabolic utilization was used to explore changes to metabolic organization associated with ITA-MP uptake (FIG. 25). This method measures protein synthesis and corresponding exogenous puromycin incorporation as a proxy for whole-cell metabolic rate in the presence of the metabolic toxins 2-deoxyglucose (2DG, a glycolytic inhibitor) or oligomycin (oxidative electron transport chain inhibitor) (FIG. 25A). ITA-MP-treated BMDM displayed a significantly higher puromycin mean fluorescence intensity (MFI) under oligomycin treatment compared to LPS-stimulated and unstimulated controls, indicating reduced dependence on oxidative phosphorylation (FIG. 25B-C). Correspondingly, 2DG decreased puromycin MFI significantly in the same treatment, further indicating primarily glycolytic dependence.

Despite these metabolic shifts, gene expression analysis of key glycolytic markers (Glut-1 and HK-1) showed a slight upregulation of Glut-1 and no significant differences in HK-1 expression between BMDM treated with ITA-MP and LPS media controls (FIG. 25D-E).

### Discussion

ITA has garnered considerable attention as an effective regulator of innate immunity, making it a compelling candidate for the development of anti-inflammatory therapeutics. However, effective delivery of ITA has proven challenging to realize with traditional systemic approaches based on its low absolute potency and barriers to uptake. This limitation extends to its derivatives, such as 4OI, which are membrane-permeable but do not fully recapitulate the endogenous effects of ITA. Here, we have demonstrated the development of degradable ITA-based polymer microparticles (ITA-MPs) that can achieve sustained and controlled delivery of ITA directly to macrophages, overcoming some of the challenges associated with systemic administration. ITA-MPs offer tunability as a drug delivery system, as particle size, degradation rates, and ITA release can be adjusted based on the synthesis and processing conditions, making them a promising platform for local inflammatory control.

Phagocytic BMDMs exhibited ready uptake of ITA-MPs but mortality at MP concentrations was low, demonstrating a window of over two orders of magnitude in concentration where phenotypic impacts of the material could be probed without causing a level of cell mortality that would make the platform untenable in vivo. In experiments probing BMDM function, phagocytosed ITA-MPs regulated cytokine production and antigen presentation. ITA-MP regulation of BMDM appeared to be dependent on the degree of particle uptake, as macrophages with higher internalization of ITA particles exhibited reduced levels of IL-6, IL-12, and MHC-II expression, suggesting that intracellular ITA release by MP degradation shifts macrophages toward an anti-inflammatory state, consistent with increased localized itaconate release. While MHC-II, which plays a key role in antigen presentation and immune activation, was initially upregulated following ITA-MP treatment, macrophages with higher particle uptake displayed a reduction in MHC-II expression, suggesting a shift toward a more tolerogenic phenotype. This finding has important implications for the use of ITA-MPs in conditions where immune regulation is desirable, such as chronic inflammation or autoimmunity, where controlled downregulation of antigen presentation may help mitigate excessive immune responses.

Mechanistically, ITA-mediated inhibition of SDH and its downstream effects on metabolic reprogramming are central to its anti-inflammatory properties. By disrupting the TCA cycle and reducing succinate-driven ROS production, ITA dampens pro-inflammatory signaling and promotes a metabolic shift toward glycolysis. Metabolic analyses recapitulated this metabolic shift in ITA-MP-treated macrophages, which exhibited reduced oxidative phosphorylation and increased reliance on glycolysis, consistent with marked inhibition of SDH and resultingly TCA flux. Interestingly, transcript abundance of key glycolysis-associated genes Glut-1 and HK-1, although ubiquitously and robustly enhanced by LPS stimulation, exhibited either no change or minor changes based on the presence or concentration of ITA-MPs. Based on the degree of functional effect of treatment afforded by SCENITH, these results suggest that the metabolic regulation induced by ITA-MP treatment is largely either on an expressional or post-translational level. The demonstration of ITA-MP-enabled uptake and selective metabolic regulation in activated phagocytes suggests a specific and targeted immunoregulatory platform that bypasses the traditional challenges of ITA-based therapeutic candidates. The benefit of a degrading delivery vehicle that concomitantly serves as the active therapeutic allows for the optimization of latency, sustained release, release rate, or the incorporation of complementary therapeutics. Future directions include the demonstration of functional immunomodulatory benefit in situ in models of chronic inflammation, such as the foreign body response or local autoimmune lesions.

### Experimental Section

### Materials

DMI, deuterated chloroform (CDCl₃), tin (II) 2-ethylhexanoate, rhodamine 6G, polyvinyl alcohol (PVA, MW 89,000-98,000, 99⁺% hydrolyzed), lipopolysaccharide (LPS), 4OI, bovine serum albumin (BSA), dimethyl sulfoxide (DMSO), and puromycin were purchased from Sigma Aldrich (St. Louis, MO). Anti-puromycin (clone 12D10, AF647) was purchased from Millipore Sigma (Burlington, MA). Dichloromethane (DCM) was purchased from VWR Chemicals (Mississauga, ON). Dodecanediol (DoD), Hoechst 33342, SYTOX Green Nucleic Acid Stain, and PCR primers (Glut-1, HK-1) were purchased from Thermo Scientific (Waltham, MA). 4-methoxyphenol (MEHQ) was purchased from RCI America (Edison, NJ). RPMI 1640 Medium with L-glutamine, Fetal Bovine Serum (FBS), Dulbecco's phosphate buffered saline (DPBS), penicillin/streptomycin solution, and EDTA were purchased from Corning (Corning, NY). ACK lysis buffer was purchased from Quality Biological (Gaithersburg, MD). Macrophage colony-stimulating factor (M-CSF), Zombie NIR Fixable Dye, TruStain FcX, monocyte blocker, anti-CD86 (clone GL-1, BV421), FOXP3 fixation/permeabilization solutions, anti-TNFα (clone MP6-XT22, BV605), anti-IL12 p40 (clone C15.6, PE-Cy7), and anti-F4/80 (clone BM8, BV605) were purchased from BioLegend (San Diego, CA). Itaconic acid (≥99.0%) and 2-deoxyglucose (2-DG) were purchased from TCI America (Portland, OR). Chloroform and tetrahydrofuran (THF) were purchased from Fisher Scientific (Waltham, MA). SuperScript IV VILO Master Mix was purchased from Invitrogen (Carlsbad, CA). Anti-MHCII (clone M5/114.15.2, BV786) and anti-IL6 (clone MP5-20F3, APC) were purchased from BD Biosciences (Franklin Lakes, NJ). Oligomycin A and harringtonine were purchased from Abcam (Cambridge, UK). All materials were used as received unless otherwise described.

### Polymer Synthesis and Characterization

ITA-based polymer was synthesized by bulk melt condensation using a 1:1 molar ratio of DMI and DoD with a 10 g basis of DMI. MEHQ was used as an inhibitor at 0.5 weight percent of reagents and tin (II) 2-ethylhexanoate as a catalyst at 0.01 mol mol⁻¹ ester bond. The reaction proceeded for 6 hours at 130° C. under nitrogen purge with overhead stirring at 200 rpm, followed by 18 hours under vacuum pressure stirring at 200 rpm. The polymer was purified twice through precipitation in −80° C. methanol and dried at room temperature for 48 h.³²

Material purity was assessed using proton nuclear magnetic spectroscopy (¹H-NMR). Samples were dissolved in deuterated CDCl₃ at a concentration of approximately 10 mg mL⁻¹, and recorded on a Bruker Avance NEO operating at a frequency of 500 MHz. Chemical shifts (δ) were reported in parts per million (ppm) relative to the residual proton signal of CDCl₃ at 7.26 ppm. Peak integration was performed to calculated percent esterification based on the following formulas:

$$ITA esterified \% = ( 1 - \frac{( f + g )}{3 b} ) \times 100 \% \qquad ( 1 )$$

$$OD esterified \% = ( 1 - \frac{2 h}{e} ) \times 100 \% \qquad ( 2 )$$

Polymer molecular weight was analyzed via gel permeation chromatography (GPC) using an Agilent 1260 Infinity Multi-Detector system fitted with two PLgel 5 μm mixed-D columns (Agilent Technologies) in series. Samples were dissolved in THF (2 mg mL⁻¹), filtered using 0.22 μm PTFE or Nylon syringe filters, and assessed at 1 mL min⁻¹ at 50° C. Calibration was performed using EasiVial PS-L standards and a 28.9 kDa nominal Mp standard, and the results were analyzed using Agilent GPC-SEC Software to determine the number-average molecular weight (Mn), weight-average molecular weight (Mw), and dispersity (D=Mw/Mn). Melting point was determined by packing the solid polymer into a glass capillary and measurement using a Mel-Temp apparatus equipped with a mercury thermometer.

### Quantification of Degradation Release of Itaconate

Soluble release of IA from poly(ITA-DoD) was quantified following incubation of poly(ITA-DoD) particulate (1 g mL⁻¹) in MilliQ water (15 MΩ cm⁻¹) under agitation (300-400 rpm) for up to 48 h (12, 24, and 48 h samples). Supernatant was collected and flash frozen in liquid nitrogen while waiting further analysis. Degradation supernatant was warmed to RT, vigorously vortexed to resolubilize any precipitates, and passed through a 0.2 μm PTFE filter to remove any insoluble particulates. Quantification of IA release was conducted via UV absorbance at 264 nm on an HPLC with metabolite confirmation by tandem MS (LC/MS). Calibration curves for accurate quantification of IA were prepared ranging from 0.01 mmol L⁻¹ to 10 mmol L⁻¹. All samples and standards were run on an Agilent LC/MSD equipped with an Agilent C-18 ZORBAX reverse phase column and appropriate guard column run at 1.5 mL min⁻¹ using MilliQ water and acetonitrile (both containing 0.1% (w/v) formic acid) as solvents. The gradient was run over 10 min from 2-30% (v/v) acetonitrile. Prior to each sample injection set, an IA standard at 5 mmol L⁻¹ concentration was submitted to control for between-run variability in the performance of the HPLC. Using Agilent OpenLab Data Analysis software, IA was quantitated by the area under the curve on the 264 nm absorbance elution peak.

### Microparticle Synthesis And Characterization

The poly(ITA-DoD) polymer microparticles (poly(ITA-DoD) MPs) were synthesized using a water-in-oil-in-water (w/o/w) emulsion method³⁵. Poly(ITA-DoD) polymer material (50 mg) was dissolved in 4 mL of DCM with 4 mg of Rhodamine 6G, followed by homogenization (rotor-stator) with 60 mL of 2% (w/v) PVA solution for 3 minutes at 20 000 rpm to create the primary emulsion. Particles were also fabricated at 10 000 and 30 000 rpm to assess size changes with speed of homogenization. An additional 40 mL of 1% PVA was added to the primary emulsion and stirred overnight to allow for organic solvent evaporation. Poly(ITA-DoD) MPs were concentrated using centrifugation (10 min, 10 000×g), washed (3×) with deionized distilled water, flash frozen and lyophilized (24 h, −60° C., 57 kPa) for storage (−20° C.) for future experiments. Characterization of ITA-MP structure was assessed using scanning electron microscopy (SEM). ITA-MPs were resuspended in MilliQ water, pipetted onto a glass slides, and left to dry overnight in a fume hood. Dried material from the slides was attached to SEM stubs with carbon tape for gold sputter-coating. SEM images were obtained using a Hitachi S-4700 FE Scanning Electron Microscope. Particle diameters were manually measured using ImageJ (US National Institutes of Health, Bethesda, MD).

### Bone Marrow-Derived Monocyte Isolation and Culture

Bone marrow was isolated from the hindlimbs of C57BL/6 mice (male and female, Charles River Laboratories), in accordance with protocol approved by the University Committee on Laboratory Animals (Dalhousie University). Animals were euthanized with carbon dioxide gas before hind limbs were removed and collected in complete RPMI media (RPMI 1640+L-glutamine (2 mmol L⁻¹), 10% FBS, 1% penicillin/streptomycin). Bones were debrided of soft tissue, rinsed in 70% ethanol, washed with PBS, then sectioned and flushed with complete RPMI media to collect bone marrow. Bone marrow was homogenized using a pestle, filtered through a 70 μm cell strainer, concentrated (centrifuged at 300×g, 5 min), and treated with ACK lysis buffer (1 min at RT, 1 mL mouse⁻¹) to remove red blood cells prior to differentiation. Cells were plated at 10 million cells per 8.8 cm-diameter plate (60.8 cm²) in complete RPMI media (10 mL) and differentiated to bone marrow derived macrophages (BMDM) by addition of 20 ng mL⁻¹ of murine M-CSF for 7 days, with 50% media changes on day 3 and 5.

### Particle Toxicity Analysis

Particle cytotoxicity against BMDM was characterized through temporal live cell imaging using a BioTek Cytation 1 and BioSpa imaging system. Cells were treated for 24 h at 50 000 cells well⁻¹ in a 96 well plate). BMDM were stained with Hoechst 33342 (2 ug mL⁻¹, 30 minutes) then subsequently treated a serial dilution of poly(ITA-DoD) particles (1, 0.5, 0.25, 0.1, 0.05, 0.025, 0.01, 0.005, 0.001 mg mL-1) prepared in complete RPMI media with M-CSF (20 ng mL⁻¹) and LPS (100 ng mL⁻¹), or a particle free media control. Wells were imaged every 6 hours to quantify changes in cell count. At 24 h, cell culture media was removed, cells were washed (3×) with PBS, and stained with SYTOX green nucleic acid stain (2 μmol L⁻¹). Imaging was performed following toxicity staining to capture cell number and associated death. Imaging analysis was performed using Agilent BioTek Gen5 Software. Cell counts calculated in BioTek Gen5 using primary masking of the nucleus (Hoechst 33342) in the DAPI channel. Secondary masking in the GFP channel (SYTOX) were constrained to the primary mask and thresholds were set based on untreated cells respectively to generate dead and live cell counts. Cytotoxicity was then calculated as percent cell death using the dead count compared to the live count and overall cell count using BioTek Gen5.

### Gene Expression

ITA-MP regulation of macrophage inflammation was assessed using BMDM (1 million cells/9.6 cm²) treated simultaneously with ITA-MPs (0.1 mg million cells 1) for an identical treatment time (24 hr) at concentrations of, 5 mmol L⁻¹ soluble itaconate (ITA), and 0.125 mmol L⁻¹ 4OI with 100 ng mL⁻¹ of LPS. Following medium removal, 750 μL of Trizol per well was added and the subsequent lysate was stored at −80° C. until further RNA extraction. Chloroform (200 μL) was added to each tube of Trizol lysate. After centrifugation (12 000×g, 15 min, 4° C.), the clear aqueous RNA containing phase was transferred for RNA extraction using the Qiagen RNeasy Plus Mini Kit. cDNA was synthesized using SuperScript IV VILO Master Mix according to manufacturer directions and gene expression was quantified using RT-qPCR. Primers used include Glut-1 (fwd:GCTGTGCTTATGGGCTTCTC, rev:CACATACATGGGCACAAAGC) and HK-1 (fwd:CCATGCGGCTCTCTGATG, rev: GGACAAAGGTTGGCAGCATC).

### Flow Cytometry Staining and Analysis

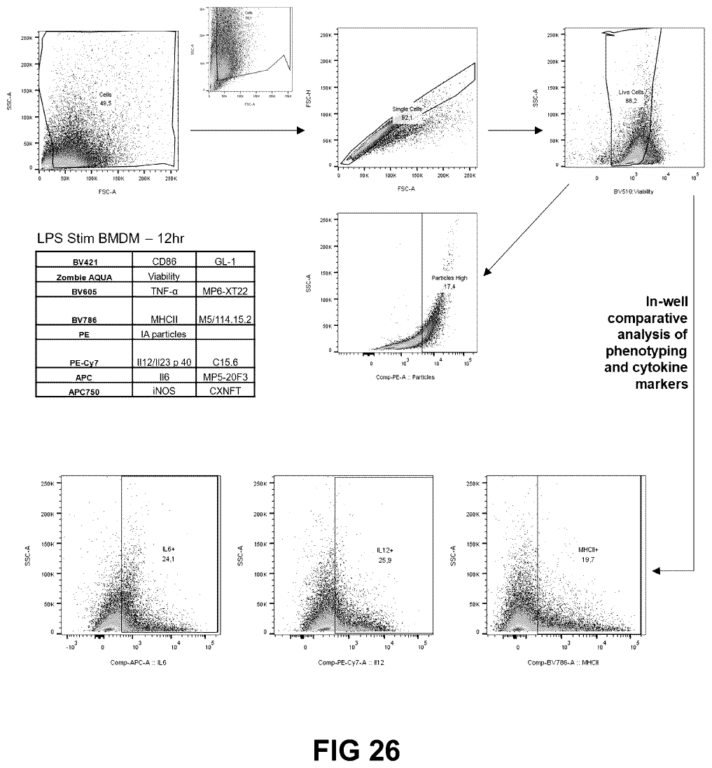

BMDM (1 million cells/9.6 cm²) were treated simultaneously with ITA-MPs (0.1 mg million cells⁻¹) for an identical treatment time (12 hr) at concentrations of 5 mmol L⁻¹ soluble itaconate (ITA), and 0.125 mmol L⁻¹ 4OI with 100 ng mL⁻¹ of LPS. Cells were trypsinized, scraped and pelleted at 300×g for 5 min, and transferred to a 96-well round-bottom polystyrene plate for staining. Cells were stained with Zombie NIR Fixable Dye (1:1000 dilution in DPBS) for 30 minutes at 4° C. in the dark, washed twice with 1×DPBS solution containing 1% (w/v) bovine serum albumin (BSA), and 1 mmol L⁻¹ EDTA (FACS Buffer). Surface antibody staining was performed with anti-CD86 (1:200) and anti-MHCII (1:500) in FACS buffer containing TruStain FcX (1:50) and Monocyte Blocker (1:50) for 45 min at 4° C. in the dark. Cells were then washed twice with FACS buffer, fixed and permeabilized using the Biolegend FOXP3 fixation/permeabilization kit in accordance with manufacturer protocols. Briefly, cells were fixed for 30 min in 200 μL of fixation/permeabilization buffer at RT, Intracellular staining of anti-TNFα (1:200), anti-IL6 (1:200) and anti-IL12 p40 (1:500 was performed in permeabilization buffer with TruStain FcX (1:50) for 30 min at RT. Cells were then washed with FACS and resuspended in DPBS for collection on the cytometer. Flow cytometry data was collected using a BD Celesta, and analysis was conducted in FlowJo v.10.9.1 (FIG. 26), with fluorescence minus-one controls (FMOs) prepared for each marker enabling effective gating of positive populations.

### Single-Cell Functional Metabolic Profiling

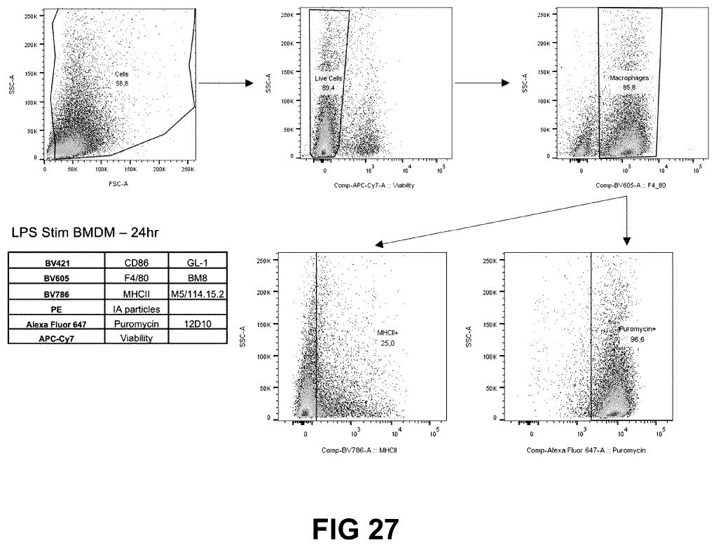

Live samples were profiled in single-cell format for metabolic function by flow cytometry using methods adapted from the validated SCENITH workflow¹⁴¹. Briefly, protein synthesis (measured by exogenous puromycin incorporation) correlates to whole-cell metabolic rate. Single-cell puromycin content is assessed across differential application of specific metabolic inhibitors to determine relative metabolic pathway dependencies of cells positively identified with concurrent standard flow cytometric expressional profiling. Samples were divided into four groups and plated in a polystyrene tissue-culture plate at 1×10⁶ cells per well. After 24 h of stimulation, for each sample, each well received a media change (1 mL RPMI 1640 with 10% FBS and 1% P/S) containing a metabolic inhibitor. For each sample, one group was treated with oligomycin A (1 mmol L⁻¹) which was used to inhibit ATP production via oxidative phosphorylation. One group received 2-DG (100 mmol L⁻¹) treatment, which was used as a glycolytic inhibitor. One group received harringtonine (2 μg mL⁻¹) treatment, which was used as a negative control to inhibit protein synthesis. DMSO (1:1000) was used as a vehicle control in the final well. Samples were incubated with metabolic inhibitors at 37° C. for 30 min. To monitor alterations to protein synthesis rates following metabolic inhibition, puromycin (10 μg mL⁻¹) was introduced and samples were incubated for an additional 30 minutes. Samples were washed with PBS and analyzed by flow cytometry as previously described. Cells were surface stained with anti-F4/80 (1:375) and anti-MHCII (1:500), then stained intracellularly with anti-puromycin (1:1000). Data was collected on a BD Celesta cytometer, and analyzed with FlowJo v.10.9.1 (FIG. 27).

### Statistical Analysis

Normality and equality of variance were tested using Graphpad Prism 8.0. Two-way ANOVA followed by pairwise comparisons with Tukey's multiple comparisons test method were used to determine the statistical significance and assess the interactive effects of factors in FIGS. 23B and 25C. One-way ANOVA followed by pairwise comparisons with Tukey's multiple comparisons test method were used to determine the statistical significance and assess the interactive effects of factors in FIGS. 23A, 24, and 25E-G.

### Tables

TABLE 1
¹H NMR data for itaconate content in PICO materials.
Esterification percentage compares the selectivity of acid
end groups to esterification. Yield of unsaturated itaconate
bond defines maintenance of pendant ITA unsaturated
groups in polymerization

| | Monomer | | % Esterified | | | |
|---|---|---|---|---|---|---|
| | Feed (%) | | Peak | Peak | | Unsaturated |
| Polymer | TEC | DMI | C | D | Overall | Yield (%) |
| 1 | 50.0 | 50.0 | 34.4 | 54.5 | 44.4 | 104.6 |
| 2 | 50.0 | 50.0 | 30.6 | 63.6 | 47.0 | 102.5 |
| 3 | 50.0 | 50.0 | 33.7 | 50.7 | 42.2 | 103.1 |
| 4 | 66.0 | 33.0 | 22.6 | 41.0 | 31.8 | 103.5 |
| 5 | 66.0 | 33.0 | 23.7 | 41.0 | 32.3 | 102.0 |
| 6 | 66.0 | 33.0 | 40.4 | 51.8 | 46.1 | 102.5 |
| 7 | 66.0 | 33.0 | 29.9 | 47.6 | 38.7 | 105.2 |
| 8 | 57.0 | 43.0 | 27.9 | 49.9 | 38.9 | 103.0 |
| 9 | 57.0 | 43.0 | 22.9 | 43.8 | 33.3 | 101.5 |
| 10 | 57.0 | 43.0 | 30.7 | 55.4 | 43.1 | 102.5 |
| 11 | 57.0 | 43.0 | 34.4 | 53.4 | 43.9 | 105.2 |

TABLE 2
Polymer reaction combinations with variation in
monomer feed ratio and reaction time

| | Feed Ratio | |
|---|---|---|
| Polymer | DMI:TEC:OD | Reaction |
| Code | (mol:mol) | Time (hr) |
| 1 | 1:1:2 | 2 + 2.5 |
| 2 | High ITA | 2 + 3 |
| 3 | | 2 + 3.5 |
| 4 | 1:2:3 | 2 + 2 |
| 5 | Low ITA | 2 + 2.5 |
| 6 | | 2 + 3 |
| 7 | | 2 + 3.5 |
| 8 | 3:4:7 | 2 + 2 |
| 9 | Moderate ITA | 2 + 2.5 |
| 10 | | 2 + 3 |
| 11 | | 2 + 3.5 |

TABLE 1
Summarized NMR analysis data indicating polymer
composition and esterification

| | | | | | | | | Monomer | |
|---|---|---|---|---|---|---|---|---|---|
| | Monomer | | Polymer | | | | | Incorporation | |
| | Feed (%) | | Content (%) | | % Esterified | | | Yield (%) | |
| Polymer | TEC | DMI | TEC | DMI | OD | TEC | DMI | TEC | DMI |
| 1 | 50.0 | 50.0 | 71.3 | 30.0 | 74.0 | 44.0 | 44.4 | 142.5 | 59.9 |
| 2 | 50.0 | 50.0 | 51.4 | 43.4 | 69.8 | 54.1 | 47.0 | 102.7 | 86.9 |
| 3 | 50.0 | 50.0 | 57.7 | 50.5 | 76.3 | 55.1 | 42.2 | 115.5 | 101.0 |
| 4 | 66.0 | 33.0 | 82.7 | 33.0 | 71.2 | 39.3 | 31.8 | 125.4 | 100.1 |
| 5 | 66.0 | 33.0 | 82.7 | 33.9 | 72.2 | 40.3 | 32.3 | 125.3 | 102.6 |
| 6 | 66.0 | 33.0 | 69.1 | 26.8 | 75.5 | 48.2 | 46.1 | 104.7 | 81.3 |
| 7 | 66.0 | 33.0 | 83.1 | 25.4 | 76.9 | 42.4 | 38.7 | 125.9 | 76.9 |
| 8 | 57.0 | 43.0 | 71.3 | 36.9 | 71.5 | 42.9 | 38.9 | 125.1 | 85.9 |
| 9 | 57.0 | 43.0 | 66.9 | 42.8 | 66.8 | 44.0 | 33.3 | 117.4 | 99.6 |
| 10 | 57.0 | 43.0 | 71.5 | 29.2 | 73.0 | 46.1 | 43.1 | 125.4 | 67.9 |
| 11 | 57.0 | 43.0 | 69.0 | 29.1 | 74.8 | 46.0 | 43.9 | 121.1 | 67.6 |

TABLE 4
GPC results for the molecular weight using triple detection,
refractive index, light scattering (LS) at 90° and 15° and
viscosity. Data are presented as average of duplicate runs.

| Polymer | Mn (Da) | Mw (Da) | D |
|---|---|---|---|
| 1 | 2845 | 18557 | 6.55 |
| 2 | 831 | 3593 | 4.40 |
| 3 | 1299 | 3825 | 2.94 |
| 4 | 2100 | 4308 | 2.05 |
| 5 | 1640 | 3305 | 2.10 |
| 6 | 5597 | 43858 | 7.89 |
| 7 | 2759 | 16763 | 6.08 |
| 8 | 1202 | * | * |
| 9 | 3102 | 5479 | 1.81 |
| 10 | 2411 | 12438 | 5.28 |
| 11 | 2456 | 19599 | 7.98 |

*Polymer 8 has a very low light scattering signal which prevented accurate calculation of the Mw.

TABLE 5
Spectral range for labelled ¹H NMR peaks

| Label | Range |
|---|---|
| A₁ | 6.35-6.30 |
| A₂ | 5.73-5.68 |
| B | 4.28-4.00 |
| C | 3.78-3.75 |
| D | 3.70-3.67 |
| E | 3.66-3.59 |
| F | 3.36-3.31 |
| G | 2.92-2.75 |
| H | 1.72-1.50 |
| I | 1.40-1.20 |

### Supplemental Methods

### Derivation of ¹H NMR Assessment Equations

To perform assessment of ¹H NMR spectra for polymer content according to functional group and esterification, equations were developed to delineate peak overlap. Peaks are defined according to FIG. 2A. Peaks are further confirmed through spectra for each monomer, triethyl citrate (TEC) (FIG. 14A), dimethyl itaconate (DMI) (FIG. 14B), and 1,8-octanediol (OD) (FIG. 14C). Given the potential for water contamination at peak H (1.56 ppm in deuterated chloroform)¹⁴², we used a system of equations to separate the contributions of peak I integration as a calculation basis. Overlap of peaks is seen between TEC endgroups and OD at peak B (TEC endgroups: B_{TEC}, and 1,8-octanediol: B_{OD}), and peak I (TEC endgroups: I_{TEC}, and 1,8-octanediol: I_{OD}). Therefore, a system of equations was developed using the relationship between these peaks to allow for assessment.

First, we defined the following:

$$I = {I}_{T E C} + {I}_{O D}$$

$$B = {B}_{T E C} + {B}_{O D}$$

The expected proton ratio of citrate content (I_{TEC}:9 protons/molecule, B_{TEC}:6 protons/molecule) and OD (I_{OD}:8 protons/molecule, B_{OD}+E: 4 protons/molecule) gives the following,

$${I}_{O D} = 2 ( {B}_{O D} + E )$$

$${I}_{T E C} = \frac{3}{2} {B}_{T E C}$$

### Substituting,

$${B}_{O D} = B - \frac{2}{3} {I}_{T E C}$$

$${I}_{O D} = 2 ( B - \frac{2}{3} {I}_{T E C} + E )$$

$${I}_{T E C} = I - 2 ( B - \frac{2}{3} {I}_{T E C} + E )$$

giving the equation,

$${I}_{T E C} = 3 [ 2 ( B + E ) - I ] \qquad ( 7 )$$

This allowed us to calculate I_{OD}:

$${I}_{O D} = I - {I}_{T E C} \qquad ( 8 )$$

Using equations 7 and 8, we then had enough independent equations to use the/peak in calculations summarized in the manuscript. Here, we describe the derivation of each equation with as it corresponds to peaks identified in FIG. 13A.

### Determination Of Polymer Content

To assess the integrated acid monomer (TEC/DMI) content in the polymer (Citrate/ITA), we compared the peak integration values of respective backbone proton NMR peaks for ITA (Peak F, 2 protons/molecule) and citrate (Peak G, 4 protons/molecule) to a basis of the OD (Peak I_{OD}, 8 protons/molecule) content in the polymer. This allowed for the development of Equations 1 & 2 that describe the acid content by percentage of the overall alcohol content:

$$ITA polymer content = \frac{4 F}{{I}_{OD}} \times 100 \% \qquad ( 1 )$$

$$Citrate polymer content = \frac{2 G}{{I}_{OD}} \times 100 \% \qquad ( 2 )$$

To calculate the yield of monomer incorporation, we used the calculated polymer content in comparison to the respective feed ratio of DMI and TEC,

$$Monomer incorporation Yield = \frac{polymer content}{monomer feed} \times 100 \% \qquad ( 3 )$$

As an indicator of monomer incorporation in the polymer chain, we next looked to assess the esterification of monomer endgroups as a measure of polymerization through condensation of each monomer group into the polymer chain. First, we defined the percentage of free endgroups for each of the monomers:
For citrate, we compared the ratio of I_{TEC} to G, which would theoretically be 9:4 protons if no end groups were esterified,

$${Endgroups}_{c i t r a t e} = \frac{4}{9} \frac{{I}_{T E C}}{G} \qquad ( 9 )$$

For ITA, we compared C+D to F, which would theoretically be 6:2 protons if no end groups were esterified,

$${Endgroups}_{ITA} = \frac{( C + D )}{3 F} \qquad ( 10 )$$

For OD, we compared E to I_{OD}, which would theoretically be 4:8 protons if no end groups were esterified,

$$Endgroups = \frac{2 E}{{I}_{O D}} \qquad ( 11 )$$

Then, to determine the degree of esterification, we subtracted the ratio of endgroups from 1 and determined a percentage of esterified endgroups for each moiety,

$$\% {Esterified}_{c i t r ate} = ( 1 - \frac{4}{9} \frac{{I}_{T E C}}{G} ) \times 100 \% \qquad ( 4 )$$

$$\% {Esterified}_{T A} = ( 1 - \frac{( C + D )}{3 F} ) \times 100 \% \qquad ( 5 )$$

$$\% {Esterified}_{OD} = ( 1 - \frac{2 E}{{I}_{O D}} ) \times 100 \% \qquad ( 6 )$$

Finally, to confirm the maintenance of unsaturated pendant group on the itaconate functional groups in PICO materials equation 12 was used.

$$Maintenance of ITA unsaturated group = \frac{( {A}_{1} + {A}_{2} )}{F} \qquad ( 12 )$$

We used this calculation method to develop the calculated values presented in Table 1, Table 3 and FIG. 13 in the main text.

### FTIR Measurement

Polymers were characterized using ATR-FTIR (Perkin Elmer Spectrum One). 32 scans from 4000 to 550 cm⁻¹ were completed at a resolution of 4 cm⁻¹ and corrections for ATR, baseline and smoothing were performed (Spectrum, Perkin Elmer).

### Molecular Weight Assessment

The molecular weight was determined by gel permeation chromatography with a Viscotek GPCmax, using a triple detection configuration with a VE 3580 RI Detector to measure the refractive index (RI) and Viscotek 270 Dual Detector for light scattering (LS) and viscosity measurements. The refractive index increment (dn/dc) was determined using a Wyatt OptiLab rEx. For dn/dc determination the polymer was dissolved in tetrahydrofuran (THF) at five concentrations ranging from 0.2 mg/mL to 15 mg/mL. The dn/dc values were measured for three polymers (2, 6, and 10) to ensure the change in monomer feed did not change the dn/dc value. The three values were in good agreement with an average of 0.0766±0.005 mL/g, which was used for the analysis of all the samples. Samples for GPC were prepared by dissolving the pre-polymer gels in THE (5 mg/mL).

### Cyclic Elastomeric Material Properties

For assessment of long-term mechanical stability, cyclic tensile tests were performed using methods described elsewhere^{143, 144}. Samples were generated and crosslinked as described for elastomeric tensile testing, then crosslinked polymer samples were cut into 15 mm by 4 mm rectangular strips and assembled into a mechanical tester (Bose, ElectroForce 5200 Biodynamic Test Instrument). A sample length between grips of 5 mm was maintained. Cyclic tensile tests were performed at a frequency of 1 Hz and a strain of 10% for 1500 cycles using a triangular waveform programmed into WinTest software. Cycling loading data was presented as stress and strain initialized to the start of the first cyclic loading cycle.

### REFERENCES

1 Teo, A. J. T. et al. Polymeric Biomaterials for Medical Implants and Devices. ACS Biomaterials Science & Engineering 2, 454-472, doi:10.1021/acsbiomaterials.5b00429 (2016).
2 Schmalzried, T. P. & Callaghan, J. J. Current concepts review-wear in total hip and knee replacements. JBJS 81, 115-136 (1999).
3 Busscher, H. J. et al. Biomaterial-Associated Infection: Locating the Finish Line in the Race for the Surface. Science translational medicine 4, 153rv110, doi:10.1126/scitranslmed.3004528 (2012).
4 Higgins, D. M. et al. Localized immunosuppressive environment in the foreign body response to implanted biomaterials. Am J Pathol 175, 161-170, doi:10.2353/ajpath.2009.080962 (2009).
Luan, H. H. & Medzhitov, R. Food Fight: Role of Itaconate and Other Metabolites in Antimicrobial Defense. Cell Metab 24, 379-387, doi:10.1016/j.cmet.2016.08.013 (2016).
6 Strelko, C. L. et al. Itaconic acid is a mammalian metabolite induced during macrophage activation. Journal of the American Chemical Society 133, 16386-16389, doi:10.1021/ja2070889 (2011).
7 Shin, J. H. et al. (1)H NMR-based metabolomic profiling in mice infected with Mycobacterium tuberculosis. J Proteome Res 10, 2238-2247, doi:10.1021/pr101054m (2011).
8 Sugimoto, M. et al. Non-targeted metabolite profiling in activated macrophage secretion. Metabolomics 8, 624-633, doi:10.1007/s11306-011-0353-9 (2011).
9 Ryan, D. G. et al. Coupling Krebs cycle metabolites to signalling in immunity and cancer. Nature Metabolism 1, 16-33, doi:10.1038/s42255-018-0014-7 (2018).
McFadden, B. & Purohit, S. Itaconate, an isocitrate lyase-directed inhibitor in Pseudomonas indigofera. Journal of bacteriology 131, 136-144 (1977).
11 Rittenhouse, J. W. & McFadden, B. A. Inhibition of isocitrate lyase from Pseudomonas indigofera by itaconate. Archives of biochemistry and biophysics 163, 79-86 (1974).
12 Garaude, J. et al. Mitochondrial respiratory-chain adaptations in macrophages contribute to antibacterial host defense. Nat Immunol 17, 1037-1045, doi:10.1038/ni.3509 (2016).
13 Michelucci, A. et al. Immune-responsive gene 1 protein links metabolism to immunity by catalyzing itaconic acid production. Proceedings of the National Academy of Sciences 110, 7820-7825, doi:10.1073/pnas.1218599110 (2013).
14 Naujoks, J. et al. IFNs Modify the Proteome of Legionella-Containing Vacuoles and Restrict Infection Via IRG1-Derived Itaconic Acid. PLoS Pathog 12, e1005408, doi:10.1371/journal.ppat.1005408 (2016).
15 O'Neill, L. A. J. & Artyomov, M. N. Itaconate: the poster child of metabolic reprogramming in macrophage function. Nat Rev Immunol, doi:10.1038/s41577-019-0128-5 (2019).
16 C. Zhang, M. Yang, and A. C. Ericsson, “Function of Macrophages in Disease: Current Understanding on Molecular Mechanisms,” Front Immunol, vol. 12, March 2021, doi: 10.3389/fimmu.2021.620510.
17 K. L. Wofford, B. S. Singh, D. K. Cullen, and K. L. Spiller, “Biomaterial-mediated reprogramming of monocytes via microparticle phagocytosis for sustained modulation of macrophage phenotype,” Acta Biomater, vol. 101, pp. 237-248, January 2020, doi: 10.1016/j.actbio.2019.11.021.
18 S. Inamdar et al., “Succinate based polymers drive immunometabolism in dendritic cells to generate cancer immunotherapy,” Journal of Controlled Release, vol. 358, pp. 541-554, June 2023, doi: 10.1016/j.jconrel.2023.05.014.
19 D. G. Ryan and L. A. J. O'Neill, “Krebs Cycle Reborn in Macrophage Immunometabolism,” Annu Rev Immunol, vol. 38, no. 1, pp. 289-313, April 2020, doi: 10.1146/annurev-immunol-081619-104850.
20 B. Kelly and L. A. O'Neill, “Metabolic reprogramming in macrophages and dendritic cells in innate immunity,” Cell Res, vol. 25, no. 7, pp. 771-784, July 2015, doi: 10.1038/cr.2015.68.
21 G. M. Tannahill et al., “Succinate is an inflammatory signal that induces IL-1β through HIF-1α,” Nature, vol. 496, no. 7444, pp. 238-242, April 2013, doi: 10.1038/nature 11986.
22 R. Li, P. Zhang, Y. Wang, and K. Tao, “Itaconate: A Metabolite Regulates Inflammation Response and Oxidative Stress,” 2020, Hindawi Limited. doi: 10.1155/2020/5404780.
23 C. Tang et al., “4-Octyl Itaconate Activates Nrf2 Signaling to Inhibit Pro-Inflammatory Cytokine Production in Peripheral Blood Mononuclear Cells of Systemic Lupus Erythematosus Patients,” Cellular Physiology and Biochemistry, vol. 51, no. 2, pp. 979-990, 2018, doi: 10.1159/000495400.
24 W. Qin et al., “S-glycosylation-based cysteine profiling reveals regulation of glycolysis by itaconate,” Nat Chem Biol, vol. 15, no. 10, pp. 983-991, October 2019, doi: 10.1038/s41589-019-0323-5.
25 T. Cordes, A. Lucas, A. S. Divakaruni, A. N. Murphy, P. Cabrales, and C. M. Metallo, “Itaconate modulates tricarboxylic acid and redox metabolism to mitigate reperfusion injury,” Mol Metab, vol. 32, pp. 122-135, February 2020, doi: 10.1016/j.molmet.2019.11.019.
26 S.-T. Liao, C. Han, D.-Q. Xu, X.-W. Fu, J.-S. Wang, and L.-Y. Kong, “4-Octyl itaconate inhibits aerobic glycolysis by targeting GAPDH to exert anti-inflammatory effects,” Nat Commun, vol. 10, no. 1, p. 5091, November 2019, doi: 10.1038/s41467-019-13078-5.
27 J.-L. Yang et al., “Keap1-Nrf2 signaling activation by Bardoxolone-methyl ameliorates high glucose-induced oxidative injury in human umbilical vein endothelial cells,” Aging, vol. 12, no. 11, pp. 10370-10380, June 2020, doi: 10.18632/aging.103263.
28 A. Swain et al., “Comparative evaluation of itaconate and its derivatives reveals divergent inflammasome and type I interferon regulation in macrophages,” Nat Metab, vol. 2, no. 7, pp. 594-602, May 2020, doi: 10.1038/s42255-020-0210-0.
29 C. G. Peace and L. A. J. O'Neill, “The role of itaconate in host defense and inflammation,” Jan. 18, 2022, American Society for Clinical Investigation. doi: 10.1172/JC1148548.
30 L. Y. Xie et al., “Itaconic acid and dimethyl itaconate exert antibacterial activity in carbon-enriched environments through the TCA cycle,” Biomedicine & Pharmacotherapy, vol. 167, p. 115487, November 2023, doi: 10.1016/j.biopha.2023.115487.
31 J. L. Mangal et al., “Metabolite releasing polymers control dendritic cell function by modulating their energy metabolism,” J Mater Chem B, vol. 8, no. 24, pp. 5195-5203, June 2020, doi: 10.1039/dOtb00790k.
32 Y. Shou et al., “Toward Renewable and Functional Biomedical Polymers with Tunable Degradation Rates Based on Itaconic Acid and 1,8-Octanediol,” ACS Appl Polym Mater, vol. 3, no. 4, pp. 1943-1955, April 2021, doi: 10.1021/acsapm.1c00017.
33 L. Davenport Huyer et al., “Macrophage Immunomodulation Through New Polymers that Recapitulate Functional Effects of Itaconate as a Power House of Innate Immunity,” Adv Funct Mater, vol. 31, no. 6, February 2021, doi: 10.1002/adfm.202003341.
34 A. P. Acharya et al., “A cell-based microarray to investigate combinatorial effects of microparticle-encapsulated adjuvants on dendritic cell activation,” J Mater Chem B, vol. 4, no. 9, pp. 1672-1685, 2016, doi: 10.1039/C5TB01754H.
35 J. S. Lewis et al., “Combinatorial delivery of immunosuppressive factors to dendritic cells using dual-sized microspheres,” J Mater Chem B, vol. 2, no. 17, pp. 2562-2574, May 2014, doi: 10.1039/c3tb21460e.
36 J. D. Fisher, A. P. Acharya, and S. R. Little, “Micro and nanoparticle drug delivery systems for preventing allotransplant rejection,” Clinical Immunology, vol. 160, no. 1, pp. 24-35, September 2015, doi: 10.1016/j.clim.2015.04.013.
37 S. T. Reddy, M. A. Swartz, and J. A. Hubbell, “Targeting dendritic cells with biomaterials: developing the next generation of vaccines,” Trends Immunol, vol. 27, no. 12, pp. 573-579, December 2006, doi: 10.1016/j.it.2006.10.005.
38 A. P. Acharya, J. S. Lewis, and B. G. Keselowsky, “Combinatorial co-encapsulation of hydrophobic molecules in poly(lactide-co-glycolide) microparticles,” Biomaterials, vol. 34, no. 13, pp. 3422-3430, April 2013, doi: 10.1016/j.biomaterials.2013.01.032.
39 F. Y. Han, K. J. Thurecht, A. K. Whittaker, and M. T. Smith, “Bioerodable PLGA-based microparticles for producing sustained-release drug formulations and strategies for improving drug loading,” Jun. 28, 2016, Frontiers Research Foundation. doi: 10.3389/fphar.2016.00185.
40 K. Park et al., “Injectable, long-acting PLGA formulations: Analyzing PLGA and understanding microparticle formation,” Journal of Controlled Release, vol. 304, pp. 125-134, June 2019, doi: 10.1016/j.jconrel.2019.05.003.
41 S. Rezvantalab and M. Keshavarz Moraveji, “Microfluidic assisted synthesis of PLGA drug delivery systems,” 2019, Royal Society of Chemistry. doi: 10.1039/C8RA08972H.
42 H. Keles, A. Naylor, F. Clegg, and C. Sammon, “Investigation of factors influencing the hydrolytic degradation of single PLGA microparticles,” Polym Degrad Stab, vol. 119, pp. 228-241, June 2015, doi: 10.1016/j.polymdegradstab.2015.04.025.
43 J. Yoo and Y. Y. Won, “Phenomenology of the Initial Burst Release of Drugs from PLGA Microparticles,” Nov. 9, 2020, American Chemical Society. doi: 10.1021/acsbiomaterials.0c01228.
44 J. Li, R. M. Stayshich, and T. Y. Meyer, “Exploiting sequence to control the hydrolysis behavior of biodegradable PLGA copolymers,” J Am Chem Soc, vol. 133, no. 18, pp. 6910-6913, May 2011, doi: 10.1021/ja200895s.
45 Y. Yang, N. Bajaj, P. Xu, K. Ohn, M. D. Tsifansky, and Y. Yeo, “Development of highly porous large PLGA microparticles for pulmonary drug delivery,” Biomaterials, vol. 30, no. 10, pp. 1947-1953, April 2009, doi: 10.1016/j.biomaterials.2008.12.044.
46 K. Y. HernAndez-Giottonini et al., “PLGA nanoparticle preparations by emulsification and nanoprecipitation techniques: Effects of formulation parameters,” 2020, Royal Society of Chemistry. doi: 10.1039/c9ra10857b.
47 E. Lagreca, V. Onesto, C. Di Natale, S. La Manna, P. A. Netti, and R. Vecchione, “Recent advances in the formulation of PLGA microparticles for controlled drug delivery,” Dec. 1, 2020, Springer Science and Business Media Deutschland GmbH. doi: 10.1007/s40204-020-00139-y.
48 K. L. Wofford, D. K. Cullen, and K. L. Spiller, “Modulation of macrophage phenotype via phagocytosis of drug-loaded microparticles,” J Biomed Mater Res A, vol. 107, no. 6, pp. 1213-1224, June 2019, doi: 10.1002/jbm.a.36617.
49 J. D. Fisher et al., “Treg-inducing microparticles promote donor-specific tolerance in experimental vascularized composite allotransplantation,” Proceedings of the National Academy of Sciences, vol. 116, no. 51, pp. 25784-25789, December 2019, doi: 10.1073/pnas.1910701116.
50 S. Inamdar et al., “Biomaterial mediated simultaneous delivery of spermine and alpha ketoglutarate modulate metabolism and innate immune cell phenotype in sepsis mouse models,” Biomaterials, vol. 293, February 2023, doi: 10.1016/j.biomaterials.2022.121973.
51 M. L. Ratay et al., “Treg-recruiting microspheres prevent inflammation in a murine model of dry eye disease,” Journal of Controlled Release, vol. 258, pp. 208-217, July 2017, doi: 10.1016/j.jconrel.2017.05.007.
52 M. M. C. S. Jaggarapu et al., “Alpha-ketoglutaric acid based polymeric particles for cutaneous wound healing,” J Biomed Mater Res A, vol. 111, no. 9, pp. 1372-1378, September 2023, doi: 10.1002/jbm.a.37539.
53 J. L. Mangal et al., “Short term, low dose alpha-ketoglutarate based polymeric nanoparticles with methotrexate reverse rheumatoid arthritis symptoms in mice and modulate T helper cell responses,” Biomater Sci, vol. 10, no. 23, pp. 6688-6697, 2022, doi: 10.1039/D2BM00415A.
54 M. L. Ratay, S. C. Balmert, A. P. Acharya, A. C. Greene, T. Meyyappan, and S. R. Little, “TRI Microspheres prevent key signs of dry eye disease in a murine, inflammatory model,” Sci Rep, vol. 7, no. 1, p. 17527, December 2017, doi: 10.1038/s41598-017-17869-y.
55 J. L. Mangal, N. Basu, H.-J. J. Wu, and A. P. Acharya, “Immunometabolism: An Emerging Target for Immunotherapies to Treat Rheumatoid Arthritis,” Immunometabolism, vol. 3, no. 4, October 2021, doi: 10.20900/immunometab20210032.
56 A. P. Acharya, M. J. Clare-Salzler, and B. G. Keselowsky, “A high-throughput microparticle microarray platform for dendritic cell-targeting vaccines,” Biomaterials, vol. 30, no. 25, pp. 4168-4177, September 2009, doi: 10.1016/j.biomaterials.2009.04.032.
57 Lampropoulou, V. et al. Itaconate Links Inhibition of Succinate Dehydrogenase with Macrophage Metabolic Remodeling and Regulation of Inflammation. Cell Metab 24, 158-166, doi:10.1016/j.cmet.2016.06.004 (2016).
58 Mills, E. L. et al. Itaconate is an anti-inflammatory metabolite that activates Nrf2 via alkylation of KEAP1. Nature, doi:10.1038/nature25986 (2018).
59 Bambouskova, M. et al. Electrophilic properties of itaconate and derivatives regulate the IkappaBzeta-ATF3 inflammatory axis. Nature, doi:10.1038/s41586-018-0052-z (2018).
60 Winkler, M., Lacerda, T. M., Mack, F. & Meier, M. A. R. Renewable Polymers from Itaconic Acid by Polycondensation and Ring-Opening-Metathesis Polymerization. Macromolecules 48, 1398-1403, doi:10.1021/acs.macromol.5b00052 (2015).
61 Hammerer, F., Chang, J. H., Duncan, D., Ruiz, A. C. & Auclair, K. Small molecule restores itaconate sensitivity in Salmonella enterica: a potential new approach to treat bacterial infections. Chembiochem, doi:10.1002/cbic.201600078 (2016).
62 Davies, L. C., Jenkins, S. J., Allen, J. E. & Taylor, P. R. Tissue-resident macrophages. Nat Immunol 14, 986-995, doi:10.1038/ni.2705 (2013).
63 Sadtler, K. et al. Design, clinical translation and immunological response of biomaterials in regenerative medicine. Nature Reviews Materials 1, doi:10.1038/natrevmats.2016.40 (2016).
64 Yang, S., Leong, K.-F., Du, Z. & Chua, C.-K. The design of scaffolds for use in tissue engineering. Part I. Traditional factors. Tissue engineering 7, 679-689 (2001).
65 Engelberg, I. & Kohn, J. Physico-mechanical properties of degradable polymers used in medical applications: a comparative study. Biomaterials 12, 292-304 (1991).
66 Pomerantseva, I. et al. Degradation behavior of poly(glycerol sebacate). Journal of biomedical materials research. Part A 91, 1038-1047, doi:10.1002/jbm.a.32327 (2009).
67 Hotaling, N. A., Tang, L., Irvine, D. J. & Babensee, J. E. Biomaterial Strategies for Immunomodulation. Annu Rev Biomed Eng 17, 317-349, doi:10.1146/annurev-bioeng-071813-104814 (2015).
68 Anderson, J. M., Rodriguez, A. & Chang, D. T. Foreign body reaction to biomaterials. Seminars in immunology 20, 86-100, doi:10.1016/j.smim.2007.11.004 (2008).
69 Sadtler, K. et al. Divergent immune responses to synthetic and biological scaffolds. Biomaterials 192, 405-415, doi:10.1016/j.biomaterials.2018.11.002 (2019).
70 Sasikaran, J., Ziemski, M., Zadora, P. K., Fleig, A. & Berg, I. A. Bacterial itaconate degradation promotes pathogenicity. Nature chemical biology 10, 371-377, doi:10.1038/nchembio.1482 (2014).
71 Ahn, S., Jung, J., Jang, I. A., Madsen, E. L. & Park, W. Role of Glyoxylate Shunt in Oxidative Stress Response. The Journal of biological chemistry 291, 11928-11938, doi:10.1074/jbc.M115.708149 (2016).
72 Bergman, J. M., Wrande, M. & Hughes, D. Acetate availability and utilization supports the growth of mutant sub-populations on aging bacterial colonies. PLoS One 9, el09255, doi:10.1371/journal.pone.0109255 (2014).
73 Dolan, S. K. & Welch, M. The Glyoxylate Shunt, 60 Years On. Annual Review of Microbiology 72, 309-330, doi:10.1146/annurev-micro-090817-062257 (2018).
74 Lindsey, T. L., Hagins, J. M., Sokol, P. A. & Silo-Suh, L. A. Virulence determinants from a cystic fibrosis isolate of Pseudomonas aeruginosa include isocitrate lyase. Microbiology 154, 1616-1627, doi:10.1099/mic.0.2007/014506-0 (2008).
75 ElAzzouny, M. et al. Dimethyl itaconate is not metabolized into itaconate intracellularly. The Journal of biological chemistry, doi:10.1074/jbc.C117.775270 (2017).
76 Chen, Z., Bozec, A., Ramming, A. & Schett, G. Anti-inflammatory and immune-regulatory cytokines in rheumatoid arthritis. Nat Rev Rheumatol, doi:10.1038/s41584-018-0109-2 (2018).
77 Booth, A. N., Taylor, J., Wilson, R. H. & DeEds, F. THE INHIBITORY EFFECTS OF ITACONIC ACID IN VITRO AND IN VIVO. Journal of Biological Chemistry 195, 697-702 (1952).
78 Marim, F. M., Silveira, T. N., Lima, D. S., Jr. & Zamboni, D. S. A method for generation of bone marrow-derived macrophages from cryopreserved mouse bone marrow cells. PLoS One 5, el5263, doi:10.1371/journal.pone.0015263 (2010).
79 Shrum, B. et al. A robust scoring system to evaluate sepsis severity in an animal model. BMC research notes 7, 233 (2014).
80 Huyer, L. D. et al. Biomaterial based cardiac tissue engineering and its applications. Biomedical materials 10, 034004, doi:10.1088/1748-6041/10/3/034004 (2015).
81 Lamba, N. K. Polyurethanes in biomedical applications. (Routledge, 2017).
82 Ye, H., Zhang, K., Kai, D., Li, Z. & Loh, X. J. Polyester elastomers for soft tissue engineering. Chem Soc Rev 47, 4545-4580, doi:10.1039/c8cs00161 h (2018).
83 Li, Y., Thouas, G. A. & Chen, Q.-Z. Biodegradable soft elastomers: synthesis/properties of materials and fabrication of scaffolds. RSC Advances 2, 8229, doi:10.1039/c2ra20736b (2012).
84 Langer, R. & Tirrell, D. A. Designing materials for biology and medicine. Nature 428, 487-492, doi:http://www.nature.com/nature/journal/v428/n6982/suppinfo/natureO2388_S1. html (2004).
85 Chan, B. P. & Leong, K. W. Scaffolding in tissue engineering: general approaches and tissue-specific considerations. European spine journal: official publication of the European Spine Society, the European Spinal Deformity Society, and the European Section of the Cervical Spine Research Society 17 Suppl 4, 467-479, doi:10.1007/s00586-008-0745-3 (2008).
86 Cohn, D. & Salomon, A. H. Designing biodegradable multiblock PCL/PLA thermoplastic elastomers. Biomaterials 26, 2297-2305 (2005).
87 Jeong, S. 1. et al. In vivo biocompatibilty and degradation behavior of elastic poly (I-lactide-co-ε-caprolactone) scaffolds. Biomaterials 25, 5939-5946 (2004).
88 Wang, L. S., Chen, H. C., Xiong, Z. C., Pang, X. B. & Xiong, C. D. A Completely Biodegradable Poly [(I-lactide)-co-(ε-caprolactone)] Elastomer Reinforced by in situ Poly (glycolic acid) Fibrillation: Manufacturing and Shape-Memory Effects. Macromolecular Materials and Engineering 295, 381-385 (2010).
89 Martin, D. P. & Williams, S. F. Medical applications of poly-4-hydroxybutyrate: a strong flexible absorbable biomaterial. Biochemical engineering journal 16, 97-105 (2003).
90 Wang, Y., Ameer, G. A., Sheppard, B. J. & Langer, R. A tough biodegradable elastomer. Nat Biotech 20, 602-606, doi:http://www.nature.com/nbt/journal/v20/n6/suppinfo/nbt0602-602_S1.html (2002).
91 Yang, J., Webb, A. R., Pickerill, S. J., Hageman, G. & Ameer, G. A. Synthesis and evaluation of poly(diol citrate) biodegradable elastomers. Biomaterials 27, 1889-1898, doi:10.1016/j.biomaterials.2005.05.106 (2006).
92 Amsden, B. Curable, biodegradable elastomers: emerging biomaterials for drug delivery and tissue engineering. Soft matter3, doi:10.1039/b707472g (2007).
93 Nijst, C. L. E. et al. Synthesis and Characterization of Photocurable Elastomers from Poly(glycerol-co-sebacate). Biomacromolecules 8, 3067-3073, doi:10.1021/bm070423u (2007).
94 Ifkovits, J. L., Padera, R. F. & Burdick, J. A. Biodegradable and radically polymerized elastomers with enhanced processing capabilities. Biomedical materials 3, 034104, doi:10.1088/1748-6041/3/3/034104 (2008).
95 Wang, Y., Kibbe, M. R. & Ameer, G. A. Photo-crosslinked Biodegradable Elastomers for Controlled Nitric Oxide Delivery. Biomater Sci 1, 625-632, doi:10.1039/C3BM00169E (2013).
96 Amsden, B. G., Misra, G., Gu, F. & Younes, H. M. Synthesis and characterization of a photo-cross-linked biodegradable elastomer. Biomacromolecules 5, 2479-2486 (2004).
97 Timbart, L. & Amsden, B. G. Functionalizable biodegradable photocrosslinked elastomers based on 2-oxepane-1,5-dione. Journal of Polymer Science Part A: Polymer Chemistry 46, 8191-8199, doi:10.1002/pola.23117 (2008).
98 Tran, R. T. et al. Synthesis and characterization of a biodegradable elastomer featuring a dual crosslinking mechanism. Soft matter 6, 2449-2461, doi:10.1039/C001605E (2010).
99 Davenport Huyer, L. et al. Highly Elastic and Moldable Polyester Biomaterial for Cardiac Tissue Engineering Applications. ACS Biomaterials Science & Engineering, doi:10.1021/acsbiomaterials.5b00525 (2016).
100 Gyawali, D., Tran, R. T., Guleserian, K. J., Tang, L. & Yang, J. Citric-acid-derived photo-cross-linked biodegradable elastomers. Journal of biomaterials science. Polymer edition 21, 1761-1782, doi:10.1163/092050609X12567178204169 (2010).
101 Zhao, H. & Ameer, G. A. Modulating the mechanical properties of poly(diol citrates) via the incorporation of a second type of crosslink network. Journal of Applied Polymer Science 114,1464-1470, doi:10.1002/app.30735 (2009).
102 Jabbari, E. et al. Synthesis, material properties, and biocompatibility of a novel self-cross-linkable poly(caprolactone fumarate) as an injectable tissue engineering scaffold. Biomacromolecules 6, 2503-2511, doi:10.1021/bm050206y (2005).
103 Zhang, B., Montgomery, M., Davenport-Huyer, L., Korolj, A. & Radisic, M. Platform technology for scalable assembly of instantaneously functional mosaic tissues. Science Advances 1, doi:10.1126/sciadv.1500423 (2015).
104 Lai, B. F. L. et al. InVADE: Integrated Vasculature for Assessing Dynamic Events. Advanced Functional Materials 27, doi:10.1002/adfm.201703524 (2017).
105 Zhao, Y. et al. A Platform for Generation of Chamber-Specific Cardiac Tissues and Disease Modeling. Cell, doi:10.1016/j.cell.2018.11.042.
106 Zhang, B. et al. Biodegradable scaffold with built-in vasculature for organ-on-a-chip engineering and direct surgical anastomosis. Nature materials, doi:10.1038/nmat4570 (2016).
107 Montgomery, M. et al. Flexible shape-memory scaffold for minimally invasive delivery of functional tissues. Nature materials, doi:10.1038/nmat4956 (2017).
108 Robert, T. & Friebel, S. Itaconic acid—a versatile building block for renewable polyesters with enhanced functionality. Green Chem. 18, 2922-2934, doi:10.1039/c6gc00605a (2016).
109 Pitt, C. G., Hendren, R. W., Schindler, A. & Woodward, S. C. The enzymatic surface erosion of aliphatic polyesters. Journal of Controlled Release 1, 3-14 (1984).
110 Rai, R., Tallawi, M., Grigore, A. & Boccaccini, A. R. Synthesis, properties and biomedical applications of poly(glycerol sebacate) (PGS): A review. Progress in Polymer Science 37, 1051-1078, doi:10.1016/j.progpolymsci.2012.02.001 (2012).
111 Tran, R. T., Yang, J. & Ameer, G. A. Citrate-Based Biomaterials and Their Applications in Regenerative Engineering. Annual Review of Materials Research 45, 277-310, doi:10.1146/annurev-matsci-070214-020815 (2015).
112 Serrano, M. C., Chung, E. J. & Ameer, G. A. Advances and Applications of Biodegradable Elastomers in Regenerative Medicine. Advanced Functional Materials 20, 192-208, doi:10.1002/adfm.200901040 (2010).
113 O'Neil, M. J. The Merck index: an encyclopedia of chemicals, drugs, and biologicals. (RSC Publishing, 2013).
114 Pellis, A., Hanson, P. A., Comerford, J. W., Clark, J. H. & Farmer, T. J. Enzymatic synthesis of unsaturated polyesters: functionalization and reversibility of the aza-Michael addition of pendants. Polymer Chemistry, doi:10.1039/c8py01655k (2019).
115 Brännström, S., Finnveden, M., Johansson, M., Martinelle, M. & Malmström, E. Itaconate Based Polyesters: Selectivity and Performance of Esterification Catalysts. European Polymer Journal, doi:10.1016/j.eurpolymj.2018.04.017 (2018).
116 Yang, J., Webb, A. R. & Ameer, G. A. Novel Citric Acid-Based Biodegradable Elastomers for Tissue Engineering. Advanced Materials 16, 511-516, doi:10.1002/adma.200306264 (2004).
117 Lang, N. et al. A Blood-Resistant Surgical Glue for Minimally Invasive Repair of Vessels and Heart Defects. Science translational medicine 6, 218ra216, doi:10.1126/scitranslmed.3006557 (2014).
118 Hoenig, E. et al. Mechanical Properties of Native and Tissue-Engineered Cartilage Depend on Carrier Permeability: A Bioreactor Study. Tissue Engineering Part A 19, 1534-1542, doi:10.1089/ten.tea.2012.0538 (2013).
119 Borschel, G. H., Kia, K. F., Kuzon, W. M. & Dennis, R. G. Mechanical properties of acellular peripheral nerve. Journal of Surgical Research 114, 133-139, doi:10.1016/s0022-4804(03)00255-5 (2003).
120 Riley, W. A., Barnes, R. W., Evans, G. W. & Burke, G. L. Ultrasonic measurement of the elastic modulus of the common carotid artery. The Atherosclerosis Risk in Communities (ARIC) Study. Stroke 23, 952-956 (1992).
121 Verdonk, E. D., Wickline, S. A. & Miller, J. G. Anisotropy of ultrasonic velocity and elastic properties in normal human myocardium. The Journal of the Acoustical Society of America 92, 3039-3050 (1992).
122 Venugopal, J. R. et al. Biomaterial strategies for alleviation of myocardial infarction. Journal of the Royal Society Interface 9, 1-19 (2011).
123 Eder, A. et al. Effects of proarrhythmic drugs on relaxation time and beating pattern in rat engineered heart tissue. Basic research in cardiology 109, 436 (2014).
124 Zimmermann, W.-H. et al. Engineered heart tissue grafts improve systolic and diastolic function in infarcted rat hearts. Nature medicine 12, 452 (2006).
125 Nunes, S. S. et al. Biowire: a platform for maturation of human pluripotent stem cell-derived cardiomyocytes. Nature methods 10, 781-787, doi:10.1038/nmeth.2524 (2013).
126 Lv, A., Li, Z.-L., Du, F.-S. & Li, Z.-C. Synthesis, Functionalization, and Controlled Degradation of High Molecular Weight Polyester from Itaconic Acid via ADMET Polymerization. Macromolecules 47, 7707-7716, doi:10.1021/ma5020066 (2014).
127 Tian, H., Tang, Z., Zhuang, X., Chen, X. & Jing, X. Biodegradable synthetic polymers: Preparation, functionalization and biomedical application. Progress in Polymer Science 37, 237-280, doi:10.1016/j.progpolymsci.2011.06.004 (2012).
128 Watson, E., Tatara, A. M., Kontoyiannis, D. P. & Mikos, A. G. Inherently Antimicrobial Biodegradable Polymers in Tissue Engineering. ACS Biomaterials Science & Engineering, doi:10.1021/acsbiomaterials.6b00501 (2016).
129 Montgomery, M. et al. Method for the Fabrication of Elastomeric Polyester Scaffolds for Tissue Engineering and Minimally Invasive Delivery. ACS Biomaterials Science & Engineering 4, 3691-3703, doi:10.1021/acsbiomaterials.7b01017 (2018).
130 He, W., Henne, A., Lauterbach, M. et al. Mesaconate is synthesized from itaconate and exerts immunomodulatory effects in macrophages. Nat Metab 4, 524-533 (2022). https://doi.org/10.1038/s42255-022-00565-1.
131 Chen, F., Elgaher, W. A. M., Winterhoff, M. et al. Citraconate inhibits ACOD1 (IRG1) catalysis, reduces interferon responses and oxidative stress, and modulates inflammation and cell metabolism. Nat Metab 4, 534-546 (2022). https://doi.org/10.1038/s42255-022-00577-x.
132 Zhou C, Wei Z, Yu Y, Shao S, Leng X, Wang Y, Li Y. Biobased long-chain aliphatic polyesters of 1,12-dodecanedioic acid with a variety of diols: Odd-even effect and mechanical properties. Materials Today Communications 19, 450-458 (2019). https://doi.org/10.1016/j.mtcomm.2019.05.005.
133 Zhou L, Qin P, Wu L, Li B, Dubois P. Potentially Biodegradable “Short-Long” Type Diol-Diacid Polyesters with Superior Crystallizability, Tensile Modulus, and Water Vapor Barrier. ACS Sustainable Chemistry & Engineering 9 (51), 17362-17370 (2021). DOI: 10.1021/acssuschemeng.1c0675259
134 Nelson T F, Rothauer D, Sander M, Mecking S. Degradable and Recyclable Polyesters from Multiple Chain Length Bio- and Waste-Sourceable Monomers. Angew. Chem. Int. Ed. 2023, 62, e202310729. https://doi.org/10.1002/anie.202310729
135 “Biologically-Degradable Polyester Mixture”, U.S. Pat. No. 8,003,731.
136 “Compatibilizing agent polyester polyol mixture containing the same, and hotmelt adhesive obtained from the mixture”, EP Patent No 1,321,484.137 H. Chikaura, Y. Nakashima, Y. Fujiwara, Y. Komohara, M. Takeya, and Y. Nakanishi, “Effect of particle size on biological response by human monocyte-derived macrophages,” Biosurf Biotribol, vol. 2, no. 1, pp. 18-25, March 2016, doi: 10.1016/j.bsbt.2016.02.003.
138 Liu Y, Yang G, Zou D, Hui Y, Nigam K D P, Middelberg A, Zhao C-X. Formulation of nanoparticles using mixing-induced nanoprecipitation for drug delivery. Industrial & Engineering Chemistry Research (2019). DOI: 10.1021/acs.iecr.9b04747.
139 Wang Z, Xiao M, Guo F, Yan Y, Tian H, Zhang Q, Ren S, Yang L. Biodegradable polyester-based nano drug delivery system in cancer chemotherapy: a review of recent progress (2021-2023). Front Bioeng Biotechnol. 2023 Nov. 1; 11:1295323. DOI: 10.3389/fbioe.2023.1295323.
140 Monge M, Fornaguera C, Quero C, Dols-Perez A, Calderó G, Grijalvo S, Garcia-Celma M J, Rodriguez-Abreu C, Solans C. Functionalized PLGA nanoparticles prepared by nano-emulsion templating interact selectively with proteins involved in the transport through the blood-brain barrier. Eur J Pharm Biopharm. 2020 November; 156:155-164. DOI: 10.1016/j.ejpb.2020.09.003.
141 R. J. Argüello et al., “SCENITH: A Flow Cytometry-Based Method to Functionally Profile Energy Metabolism with Single-Cell Resolution,” Cell Metab, vol. 32, no. 6, pp. 1063-1075.e7, December 2020, doi: 10.1016/j.cmet.2020.11.007.
142 Gottlieb, H. E., Kotlyar, V. & Nudelman, A. NMR chemical shifts of common laboratory solvents as trace impurities. The Journal of organic chemistry 62, 7512-7515 (1997).
143 Lin, X. et al. A viscoelastic adhesive epicardial patch for treating myocardial infarction. Nat Biomed Eng, doi:10.1038/s41551-019-0380-9 (2019).
144 Engelmayr, G. C., Jr. et al. Accordion-like honeycombs for tissue engineering of cardiac anisotropy. Nature materials 7, 1003-1010, doi:10.1038/nmat2316 (2008).

What is claimed is:

1. A particle comprising a polyester biomaterial comprising diol monomers and itaconate, wherein the diol monomers comprise at least 1,12-dodecanediol.

2. The particle of claim 1, wherein the diol monomers further comprise 1,6-hexanediol, 1,8-octanediol, 1,10-decanediol, or a combination thereof.

3. The particle of claim 1, wherein the particle is formulated for the treatment of at least one of an infection or an inflammation, or a condition resulting directly from an infection or an inflammation, in a subject.

4. The particle of claim 1, wherein the particle has a particle size ranging from about 25 nm to about 100 μm.

5. The particle of claim 1, wherein the particle is formed by homogenization of a suspension or dispersion, spraying, microfluidic manipulation, vapor deposition, condensation, emulsion, coacervation, laser lysis, milling, fracture, seeding, lithography, or sol-gel methods.

6. The particle of claim 1, wherein the polyester biomaterial forms part of a coating for a polymer surface or a metal surface, and wherein the polymer surface or the metal surface is a surface of a medical implantable device or a medical instrumentation device.

7. The particle of claim 1, wherein the polyester biomaterial is characterized by hydrolytic degradability.

8. The particle of claim 7, wherein hydrolytic degradation of the polyester biomaterial causes release of therapeutic degradation products, and wherein the degradation products comprise itaconate, an isomer of itaconate, or a combination thereof.

9. The particle of claim 8 wherein the degradation products further comprise itaconate bonded with at least one of the diol monomers, an isomer of itaconate bonded with at least one of the diol monomers, or a combination thereof.

10. The particle of claim 1, wherein the polyester biomaterial is formed by polycondensation of the diol monomers with the itaconate monomers in the presence of a radical inhibitor.

11. The particle of claim 10, wherein the polyester biomaterial is formed by polycondensation in a temperature range from about 120° C. to about 130° C. at atmospheric pressure.

12. The particle of claim 10, wherein the polyester biomaterial is formed by additional polycondensation at vacuum pressure.

13. The particle of claim 1, wherein the itaconate monomers comprise methylated itaconate.

14. The particle of claim 1, wherein the polyester biomaterial further comprises second carboxylate monomers.

15. The particle of claim 14, wherein the polyester biomaterial is formed by forming a polyester backbone including the diol monomers and the second carboxylate monomers, and reacting the polyester backbone with the itaconate monomers.

16. The particle of claim 14, wherein the polyester biomaterial is formed at atmospheric pressure at about 120° C.

17. The particle of claim 14, wherein the second carboxylate monomers comprise citrate.

18. A method of fabricating the polyester biomaterial of claim 1, the method comprising:
forming a polyester backbone including the diol monomers; and
reacting the polyester backbone with the itaconate monomers.

19. A method of treating at least one of an infection or an inflammation, or a condition resulting directly from an infection or an inflammation, in a subject, the method comprising:
providing a polyester biomaterial comprising diol monomers and at least first carboxylate monomers, wherein the first carboxylate monomers are itaconate and the diol monomers comprise at least 1,12-dodecanediol, and wherein the polyester biomaterial is in the form of a particle; and
administering the polyester biomaterial to the subject.

20. The method of claim 19, wherein the diol monomers further comprise 1,6-hexanediol, 1,8-octanediol, 1,10-decanediol, or a combination thereof.

21. The method of claim 19, further comprising the step of encapsulating an active molecule using the particle, before the administering step.

22. The method of claim 19, wherein the administering step comprises topical administration, enteral administration, or by subcutaneous, intradermal, intramuscular, intraocular, intraperitoneal, intraarticular, or intravascular injection.

23. The method of claim 19, wherein the polyester biomaterial is provided as part of a scaffold before the administering step.

24. The method of claim 22, wherein the scaffold is for a tissue patch.
